(19) United States
(12) Patent Application Publication
(10) Pub. No.: US 2007/0274921 A1
(43) Pub. Date: Nov. 29, 2007

(54) Diagnostics and therapeutics for ocular disorders

(75) Inventors: Gregory S. Hageman, Coralville, IA (US); Robert F. Mullins, Coralville, IA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Iowa Centers for Enterprise, Iowa City, IA (US)

(21) Appl. No.: 11/827,822

(22) Filed: Jul. 13, 2007

Related U.S. Application Data
(63) Continuation of application No. 09/949,261, filed on Sep. 6, 2001, now abandoned.
Application No. 09/949,261 is a continuation-in-part of application No. 09/510,230, filed on Feb. 22, 2000, now abandoned.
Application No. 09/949,261 is a continuation-in-part of application No. 09/845,745, filed on Apr. 30, 2001, now abandoned.
(60) Provisional application No. 60/120,822, filed on Feb. 19, 1999.
(60) Provisional application No. 60/120,668, filed on Feb. 19, 1999.
(60) Provisional application No. 60/123,052, filed on Mar. 5, 1999.
(60) Provisional application No. 60/200,698, filed on Apr. 29, 2000.

(51) Int. Cl. A61K 49/00; A61P 27/02; G01N 33/53

(52) U.S. Cl. 424/9.2; 424/278.1; 424/85.2; 424/9.1; 435/6; 435/7.21; 436/501; 514/789; 435/7.2

(57) ABSTRACT

The invention relates to methods for treating, preventing and diagnosing drusen-associated disorders.

### CROSS-REFERENCES TO RELATED APPLICATIONS

This application claims the benefit of priority from co-pending U.S. patent application Ser. No. 09/510,230 (filed Feb. 22, 2000) and Ser. No. 09/845,745 (filed Apr. 30, 2001), which in turn respectively claim priority to U.S. Provisional Application Ser. Nos. 60/120,822 (filed Feb. 19, 1999), 60/120,668 (filed Feb. 19, 1999), 60/123,052 (filed Mar. 5, 1999); and 60/200,698 (filed Apr. 29, 2000). The full disclosures of these applications are incorporated herein by reference in their entirety for all purposes.

### STATEMENT AS TO RIGHTS TO INVENTIONS MADE UNDER FEDERALLY SPONSORED RESEARCH AND DEVELOPMENT

Work described herein has been supported, in part, by NIH National Eye Institute grant EY1 1515. The U. S. Government may therefore have certain rights in the invention.

### BACKGROUND OF THE INVENTION

Macular degeneration is a clinical term that is used to describe a variety of diseases that are all characterized by a progressive loss of central vision associated with abnormalities of Bruch's membrane, the neural retina and the retinal pigment epithelium. These disorders include very common conditions that affect older patients (age-related macular degeneration or AMD) as well as rarer, earlier-onset dystrophies that in some cases can be detected in the first decade of life (Best F. Z., Augenheilkd., 13:199-212, 1905; Sorsby, A., et al., Br J. Opthalmol. 33:67-97, 1949; Stargardt, K., Albrecht Von Graefes Arch Klin Exp Opthalmol. 71: 534-550, 1909; Ferrell, R. E., et al., Am J. Hum Genet.35:78-84, 1983; Jacobson, D. M., et al., Ophthalmology, 96:885-895, 1989; Small, K. W., et al. Genomics 13:681-685, 1992; Stone, E. M., et al., Nature Genet. 1:246-250, 1992; Forsman, K., et al. Clin Genet. 42:156-159, 1992; Kaplan, J. S., et al. Nature Genet. 5:308-311, 1993; Stone, E. M., et al. Arch Opthalmol. 112:763-772, 1994; Zhang, K., et al. Arch Opthalmol. 112:759-764, 1994; Evans, K., et al. Nature Genet. 6:210-213, 1994; Kremer, H., et al. Hum Mol Genet. 3:299-302, 1994; Kelsell, R. E., et al. Hum Mol Genet. 4:1653-1656, 1995; Nathans, J., et al. Science 245:831-838, 1989; Wells, J., et al. Nature Genet. 3:213-218, 1993; Nichols, B. E., et al. Nature Genet. 3:202-207, 1993a; Weber, B. H. F., et al. Nature Genet. 8:352-355, 1994), the teachings of which are incorporated herein by reference. Macular degeneration diseases include, for example, age- related macular degeneration, North Carolina macular dystrophy, Sorsby's fundus dystrophy, Stargardt's disease, pattern dystrophy, Best disease, malattia leventinese, Doyne's honeycomb choroiditis, dominant drusen and radial drusen.

A number of gene loci have been reported as indicating a predisposition to macular degeneration: 1p21-q13, for recessive Stargardt's disease or fundus flavi maculatus (Allikmets, R. et al. Science 277:1805-1807, 1997; Anderson, K. L. et al., Am. J. Hum. Genet. 55:1477, 1994; Cremers, F. P. M. et al., Hum. Mol. Genet. 7:355-362, 1998; Gerber, S. et al., Am. J. Hum. Genet. 56:396-399, 1995; Gerber, S. et al., Genomics 48:139-142, 1998; Kaplan, J. et al., Nat. Genet. 5:308-311, 1993; Kaplan, J. et al., Am. J. Hum. Genet. 55:190, 1994; Martinez-Mir, A. et al., Genomics 40:142-146, 1997; Nasonkin, I. et al., Hum. Genet. 102:21-26, 1998; Stone, E. M. et al., Nat. Genet. 20:328-329, 1998); 1q25-q31, for recessive age-related macular degeneration (Klein, M. L. et al., Arch. Ophthalmol. 116:1082-1088, 1988); 2p16, for dominant radial macular drusen, dominant Doyne honeycomb retinal degeneration or Malattia Leventinese (Edwards, A. O. et al., Am. J. Ophthalmol. 126:417-424, 1998; Heon, E. et al., Arch. Ophthalmol. 114:193-198, 1996; Heon, E. et al.,. Invest. Ophthalmol Vis. Sci. 37:1124, 1996; Gregory, C. Y. et al., Hum. Mol. Genet. 7:1055-1059, 1996); 6p21.2-cen, for dominant macular degeneration, adult vitelloform (Felbor, U. et al. Hum. Mutat. 10:301-309, 1997); 6p21.1 for dominant cone dystrophy (Payne, A. M. et al. Am. J. Hum. Genet. 61:A290, 1997; Payne, A. M. et al., Hum. Mol. Genet. 7:273-277, 1998; Sokol, I. et al., Mol. Cell. 2:129-133, 1998); 6q, for dominant cone-rod dystrophy (Kelsell, R. E. et al. Am. J. Hum. Genet. 63:274-279, 1998); 6q11-q15, for dominant macular degeneration, Stargardt's-like (Griesinger, I. B. et al., Am. J. Hum. Genet. 63:A30, 1998; Stone, E. M. et al., Arch. Ophthalmol. 112:765-772, 1994); 6q14-q16.2, for dominant macular degeneration, North Carolina Type (Kelsell, R. E. et al., Hum. Mol. Genet. 4:653-656, 1995; Robb, M. F. et al., Am. J. Ophthalmol. 125:502-508, 1998; Sauer, C. G. et al., J. Med. Genet. 34:961-966, 1997; Small, K. W. et al., Genomics 13:681-685, 1992; Small, K. W. et al., Mol. Vis. 3:1, 1997); 6q25-q26, dominant retinal cone dystrophy 1 (Online Mendelian Inheritance in Man (TM). Center for Medical Genetics, Johns Hopkins University, and National Center for Biotechnology Information, National Library of Medicine (http://www3.ncbi.nlm.nih.gov/omim, (1998)); 7p21-p15, for dominant cystoid macular degeneration (Inglehearn, C. F. et al., Am. J. Hum. Genet. 55:581-582, 1994; Kremer, H. et al., Hum. Mol. Genet. 3:299-302, 1994); 7q31.3-32, for dominant tritanopia, protein: blue cone opsin (Fitzgibbon, J. et al., Hum. Genet. 93:79-80, 1994; Nathans, J. et al., Science 193:193-232, 1986; Nathans, J. et al., Ann. Rev. Genet. 26:403-424, 1992; Nathans, J. et al., Am. J. Hum. Genet. 53:987-1000, 1993; Weitz, C. J. et al., Am. J. Hum. Genet. 50:498-507, 1992; Weitz, C. J. et al., Am. J. Hum. Genet. 51:444-446, 1992); not 8q24, for dominant macular degeneration, atypical vitelliform (Daiger, S. P. et al., In ‘Degenerative Retinal Diseases’, LaVail, et al., eds. Plenum Press, 1997; Ferrell, R. E. et al., Am. J. Hum. Genet. 35:78-84, 1983; Leach, R. J. et al., Cytogenet. Cell Genet. 75:71-84, 1996; Sohocki, M. M. et al., Am. J. Hum. Genet. 61:239-241, 1997); 11p12-q13, for dominant macular degeneration, Best type (bestrophin) (Forsman, K. et al., Clin. Genet. 42:156-159, 1992; Graff, C. et al., Genomics, 24:425-434, 1994; Petrukhin, K. et al., Nat. Genet. 19:241-247, 1998; Marquardt, A. et al., Hum. Mol. Genet. 7:1517-1525, 1998; Nichols, B. E. et al., Am. J. Hum. Genet. 54:95-103, 1994; Stone, E. M. et al., Nat. Genet. 1:246-250, 1992; Wadeilus, C. et al., Am. J. Hum. Genet. 53:1718, 1993; Weber, B. et al., Am. J. Hum. Genet. 53:1099, 1993; Weber, B. et al., Am. J. Hum. Genet. 55:1182-1187, 1994; Weber, B. H., Genomics 20: 267-274, 1994; Zhaung, Z. et al., Am. J. Hum. Genet. 53:1112, 1993); 13q34, for dominant macular degeneration, Stargardt type (Zhang, F. et al., Arch. Ophthalmol. 112:759-764, 1994); 16p12.1, for recessive Batten disease (ceroid-lipofuscinosis, neuronal 3), juvenile; protein: Batten disease protein (Batten Disease Consortium, Cell 82:949-957, 1995; Eiberg, H. et al., Clin. Genet. 36:217-218, 1989; Gardiner, M. et al., Genomics 8:387-390, 1990; Mitchison, H. M. et al., Am. J. Hum. Genet. 57:312-315, 1995, Mitchison, H. M. et al., Am. J. Hum. Genet. 56:654-662, 1995; Mitchison, H. M. et al., Genomics 40:346-350, 1997; Munroe, P. B. et al., Am. J. Hum. Genet. 61:310-316, 1997; 17p, for dominant areolar choroidal dystrophy (Lotery, A. J. et al., Ophthalmol. Vis. Sci. 37:1124, 1996); 17p13-p12, for dominant cone dystrophy, progressive (Balciuniene, J. et al., Genomics 30:281-286, 1995; Small, K. W. et al., Am. J. Hum. Genet. 57:A203, 1995; Small, K. W. et al., Am. J. Ophthalmol. 121:13-18, 1996); 17q, for cone rod dystrophy (Klystra, J. A. et al., Can. J. Ophthalmol. 28:79-80, 1993); 18q21.1-q21.3, for cone-rod dystrophy, de Grouchy syndrome (Manhant, S. et al., Am. J. Hum. Genet. 57:A96, 1995; Warburg, M. et al., Am. J. Med. Genet. 39:288-293, 1991); 19q13.3, for dominant cone-rod dystrophy; recessive, dominant and ‘de novo’ Leber congenital amaurosis; dominant RP; protein: cone-rod otx-like photoreceptor homeobox transcription factor (Bellingham, J. et al., In ‘Degenerative Retinal Diseases’, LaVail, et al., eds. Plenum Press, 1997; Evans, K. et al., Nat. Genet. 6:210-213, 1994; Evans, K. et al., Arch. Ophthalmol. 113:195-201, 1995; Freund, C. L. et al., Cell 91:543-553, 1997; Freund, C. L. et al., Nat. Genet. 18:311-312, 1998; Gregory, C. Y. et al., Am. J. Hum. Genet. 55:1061-1063, 1994; Li, X. et al., Proc. Natl. Acad. Sci USA 95:1876-1881, 1998; Sohocki, M. M. et al., Am. J. Hum. Genet. 63:1307-1315, 1998; Swain, P. K. et al., Neuron 19:1329-1336, 1987; Swaroop, A. et al., Hum. Mol. Genet. In press, 1999); 22q12.1-q13.2, for dominant Sorsby's fundus dystrophy, tissue inhibitors of metalloproteases-3 (TIMP3) (Felbor, U. et al., Hum. Mol. Genet. 4:2415-2416, 1995; Felbor, U. et al., Am. J. Hum. Genet. 60:57-62, 1997; Jacobson, S. E. et al., Nat. Genet. 11:27-32, 1995; Peters, A. et al., Retina 15:480-485, 1995; Stohr, H. et al., Genome Res. 5:483-487, 1995; Weber, B. H. F. et al., Nat. Genet. 8:352-355, 1994; Weber, B. H. F. et al., Nat. Genet. 7:158-161, 1994; Wijesvriya, S. D. et al., Genome Res. 6:92-101, 1996); and Xp11.4, for X-linked cone dystrophy (Bartley, J. et al., Cytogenet. Cell. Genet. 51:959, 1989; Bergen, A. A. B. et al., Genomics 18:463-464, 1993; Dash-Modi, A. et al., Invest. Ophthalmol. Vis. Sci. 37:998, 1996; Hong, H.-K., Am. J. Hum. Genet 55:1173-1181, 1994; Meire, F. M. et al., Br. J. Ophthalmol. 78:103-108, 1994; Seymour, A. B. et al., Am. J. Hum. Genet. 62:122-129, 1998), the teachings of which are incorporated herein by reference. In addition, the world wide web site http://WWW.SPH.UTH.TMC.EDU/RETNET/disease.htm lists genetic polymorphisms for macular degenerations and for additional retinal degenerations that also may be associated with macular degeneration. However, none of the above genes or polymorphisms has been found to be responsible for a significant fraction of typical late-onset age-related macular degeneration. Although a recent report suggested that mutations in the photoreceptor ABCR rim protein cause up to 15% of AMD cases in the United States (Allikmets, et al., 1997), conflicting results have been obtained by different investigators (De La Paz, et al., 1998; Stone et al., 1998).

Age-related macular degeneration (AMD), the most prevalent macular degeneration is associated with progressive diminution of visual acuity in the central portion of the visual field, changes in color vision, and abnormal dark adaptation and sensitivity (Steinmetz, et al., 1993; Brown & Lovie-Kitchin, 1983; Brown, et al., 1986; Sunness, et al., 1985; Sunness, et al., 1988; Sunness, et al., 1989; Eisner, et al., 1987; Massof, et al., 1989; Chen, et al., 1992).

AMD is the leading cause of legal blindness in North America and Western Europe (Hyman, 1992) and has become a significant health problem as the percentage of individuals above the age of 50 increases. In the Beaver Dam, Wisconsin population, the incidence of AMD was estimated to be 9.2% for persons over the age of 40 (Klein, et al., 1995). The Framingham Eye Study found the overall incidence of AMD to be 8.8%, with a 27.9% incidence in the 75-85 year old population (Kahn, et al., 1977; Leibowitz, et al., 1980). In an Australian study, 18.5% of those over age 85 were estimated to be afflicted with AMD (O'Shea, 1996). Variations in estimated incidence are likely a result of the use of different criteria for a diagnosis of AMD in different studies, or they may result from different risk factors among the various populations studied.

Two principal clinical manifestations of AMD have been described, both of which can occur in the same patient (Green and Key, 1977). They are referred to as the dry, or atrophic, form, and the wet, or exudative, form (Sarks and Sarks, 1989; Elman and Fine, 1989; Kincaid, 1992). The most significant risk factor for the development of both forms are age and the deposition of drusen, abnormal extracellular deposits, behind the retinal pigment epithelium (RPE). In the dry form of AMD, the RPE and retina degenerate without coincident neovascularization. The -region of atrophy that results is referred to as geographic atrophy. While atrophic AMD is typically considered less severe than the exudative form because its onset is less sudden, no treatment is effective at halting or slowing its progression. In the less common, but more devastating, exudative form, neovascular “membranes” derived from the choroidal vasculature invade Bruch's membrane, leak, and often cause detachments of the RPE and/or the neural retina (Elman and Fine, 1989). This event can occur over a short period of time and can lead to rapid and permanent loss of central vision. If one eye is affected, there is a high degree of probability that the second eye will develop a choroidal neovascular membrane within five years of the initial event (Macular Photocoagulation Study, 1977). Important clinical signs of neovascular AMD include gray-green neovascular membranes, dome-shaped RPE detachments, and disciform scars (caused by proliferation of fibroblasts and retinal glial cells) which are best visualized by their hyperfluorescence on fluorescein angiography (Elman and Fine, 1989). Killingsworth et al. (1990) suggested that macrophages may participate in the breakdown of Bruch's membrane in the neovascular stage of AMD and in drusen regression, and show one electron micrograph depicting structures resembling drusen cores. Duvall and Tso (1985) showed choroidal macrophages in the region of the Bruch's membrane are involved in the removal of drusen in monkey eyes, following laser photocoagulation. Penfold and others (Penfold et al., 1985; Penfold et al., 1986; Oppenheim and Leonard, 1989) provided “circumstantial evidence . . . for the involvement of (choroidal) leukocytes, in the promotion of neovascular proliferation.” However, these data were restricted to morphological observations only and only suggest that macrophages only participate in the neovascularization stage of drusen formation.

A number of population-based studies indicate that AMD has a genetic component, based upon the examination of the rates of AMD in different racial groups and the degree of familial aggregation of AMD (Hyman, et al., 1983). For example, Caucasians appear to be at greater risk than individuals of Hispanic origin (Cruickshanks, et al., 1997). In addition, a black population on Barbados had a lower incidence of advanced AMD than the local Caucasian population (Schachat, et al., 1995). Studies involving twins and other siblings have demonstrated that, the more related two individuals are, the more likely they are to be at the same risk of developing AMD (Heiba, et al., 1994; Klein, et al., 1994; Meyers and Zacchary, 1988; Meyers, 1994; Meyers, et al., 1995; Piguet, et al., 1993; Seddon, et al., 1997; Silvestri, et al., 1994). These findings suggest that heredity contributes significantly to an individual's risk of developing AMD, but the gene(s) responsible have not been identified.

Other maculopathies, typically with an earlier onset of symptoms than AMD, have bee n described. These include North Carolina macular dystrophy (Small, et al., 1993), Sorsby's fundus dystrophy (Capon, et al., 1989), Stargardt's disease (Parodi, 1994), pattern dystrophy (armor and Byers, 1977), Best disease (Stone, et al., 1992), dominant drusen (Deutman and Jansen, 1970), and radial drusen (“malattia leventinese”) (Heon, et al., 1996). Several of these inherited disorders, including those that map to distinct chromosomal loci or for which the genes have been identified, are characterized by the presence of drusen (or other extracellular deposits in the subRPE space). Based on this information, it is likely that: (1) AMD is not a single, genetic disease, since different diseases with distinct chromosomal loci share morphologic differences (Holz, et al., 1995a; Mansergh et al., 1995; and (2) that drusen may develop as a result of a biological pathway induced by a variety of different insults, genetic or otherwise. Determining whether AMD is a genetic or an acquired disorder is problematic, since AMD may actually be several diseases, and thus defy simple categorization; indeed, both genetic and environmental factors appear to play some role in its development.

“Environmental” conditions may modulate the rate at which an individual develops AMD or the severity of the disease. Light exposure has been proposed as a possible risk factor, since AMD most severely affects the macula, where light exposure is high. (Young, 1988; Taylor, et al., 1990; Schalch, 1992). The amount of time spent outdoors is associated with increased risk of choroidal neovascularization in men, and wearing hats and/or sunglasses is associated with a decreased incidence of soft drusen (Cruickshanks, et al., 1993). Accidental exposure to microwave irradiation has also been shown to be associated with the development of numerous drusen (Lim, et al., 1993). Cataract removal and light iris pigmentation has also been reported as a risk factor in some studies (Sandberg, et al., 1994). This suggests that: 1) eyes prone to cataracts may be more likely to develop AMD; 2) the surgical stress of cataract removal may result in increased risk of AMD, due to inflammation or other surgically-induced factors; or 3) cataracts prevent excessive light exposure from falling on the macula, and are in some way prophylactic for AMD. While it is possible that dark iris pigmentation may protect the macula from light damage, it is difficult to distinguish between iris pigmentation alone and other, cosegregating genetic factors which may be actual risk factors.

Dietary factors may also influence an individual's risk of developing AMD. Anecdotal evidence from Japan suggests that the incidence of AMD, while very low 20 years ago, has increased as urban Japanese acquired a more Western diet and lifestyle (Bird, 1997). Chemical exposure (Hyman, et al., 1983), smoking (Vingerling, et al., 1996), cardiovascular disease/atherosclerosis (Hyman, et al., 1983; Vingerling, et al., 1995; Blumenkranz, et al., 1986), hypertension (Christen, et al., 1997), dermal elastotic changes in non-sun exposed skin (Blumenkranz, et al., 1986), dietary fat intake (Mares-Perlman, et al., 1995b), low concentrations of serum lycopene (Mares-Perlman, et al., 1995a), and alcohol consumption (Ritter, et al., 1995) have been identified, in some studies, as additional risk factors for the development of wet and/or dry AMD. One recent prospective dietary study found that it is often possible to increase macular pigment density and/or serum concentrations of lutein and zeaxanthin by dietary intake (Hammond, et al., 1997), although the significance of this alteration in modulating macular disease remains to be determined. Thus, dietary consumption of some vegetables, (e.g., spinach, collard greens, kale) may be inversely associated with the risk of developing AMD (Seddon, et al., 1994), an effect which is presumably due to their lutein and zeaxanthin content.

Histopathologic studies have documented significant and widespread abnormalities in the extracellular matrices associated with the RPE, choroid, and photoreceptors of aged individuals and of those with clinically-diagnosed AMD (Sarks, 1976; Sarks, et al., 1988; Bird, 1992a; van der Schaft, et al., 1992; Green and Enger, 1993; Feeney-Burns and Ellersieck, 1985; Young, 1987; Kincaid, 1992). The most prominent extracellular matrix (ECM) abnormality is drusen, deposits that accumulate between the RPE basal lamina and the inner collagenous layer of Bruch's membrane (FIG. 1). Drusen appear to affect vision prior to the loss of visual acuity; changes in color contrast sensitivity (Frennesson, et al., 1995; Holz, et al., 1995b; Midena, et al., 1994; Stangos, et al., 1995; Tolentino, et al., 1994), macular recovery function, central visual field sensitivity, and spatiotemporal contrast sensitivity (Midena, et al., 1997) have been reported.

A number of studies have demonstrated that the presence of macular drusen is a strong risk factor for the development of both atrophic and neovascular AMD (Gass, 1973; Lovie-Kitchin and Bowman, 1985; Lewis, et al., 1986; Sarks, 1980; Sarks, 1982; Small, et al., 1976; Sarks, et al., 1985; Vinding, 1990; Bressler, et al., 1994; Bressler, et al., 1990; Macular Photocoagulation Study). Pauleikhoff, et al. (1990) demonstrated that the size, number, density and extent of confluency of drusen are important determinants of the risk of AMD. The risk of developing neovascular complications in patients with bilateral drusen has been estimated at 3-4% per year (Mimoun, et al., 1990). A recent report from the Macular Photocoagulation Study Group shows a relative risk of 2.1 for developing choroidal neovascularization in eyes possessing 5 or more drusen, and a risk of 1.5 in eyes with one or more large drusen (Macular Photocoagulation Study, 1997). The correlation between drusen and AMD is significant enough that many investigators and clinicians refer to the presence of soft drusen in the macula, in the absence of vision loss, as “early AMD” (Midena, et al., 1997; Tolentino, et al., 1994), or “early age-related maculopathy” (Bird, et al., 1995). In addition to macular drusen, Lewis et al. (1986) found that the degree of extramacular drusen is also a significant risk factor for the development of AMD. A few clinical studies have shown that drusen regress and that visual acuity improves in some cases, following laser photocoagulation (Sigelman, 1991; Little, et al., 1997; Figueroa, et al., 1994; Frenneson and Nilsson, 1996). While prophylactic laser treatment may be helpful for some patients (Little, et al., 1997), it appears that other patients react adversely to laser treatment of the macula (Hyver, et al., 1997). In addition, while there may be long term benefits for the patient following photocoagulation, these may not be worth the loss of vision frequently associated with this procedure.

Drusen accumulate between the RPE basal lamina and the inner collagenous layer of Bruch's membrane. They cause a lateral stretching of the RPE monolayer and physical displacement of the RPE from its immediate vascular supply, the choriocapillaris. This displacement creates a physical barrier that may impede normal metabolite and waste diffusion between the choriocapillaris and the retina. It is likely that wastes may be concentrated near the RPE and that the diffusion of oxygen, glucose, and other nutritive or regulatory serum-associated molecules required to maintain the health of the retina and RPE are inhibited. It has also been suggested that drusen perturb photoreceptor cell function by placing pressure on rods and cones (Rones, 1937) and/or by distorting photoreceptor cell alignment (Kincaid, 1992).

The terminology most commonly used to distinguish drusen phenotypes is hard and soft (see, for example, Eagle, 1984; Lewis, et al., 1986; Yanoff and Fine, 1992; Newsome, et al., 1987; Mimoun, et al., 1990; van der Schaft, et al., 1992; Spraul and Grossniklaus, 1997), although numerous drusen phenotypes exist (Mullins & Hageman, 1999, Mol. Vision). Hard drusen are typically defined as small distinct deposits comprised of homogeneous eosinophilic material. Histologically, they are round or hemispherical, without sloped borders. Soft drusen are larger and have sloped, indistinct borders. Unlike hard drusen, soft drusen are not usually homogeneous, and typically contain inclusions and spherical profiles. An eye with many large/soft drusen is at a significantly higher risk of developing complications of AMD than is an eye with no drusen or a few, small drusen. The term “diffuse drusen,” or “basal linear deposit,” is used to describe the amorphous material which forms a layer between the inner collagenous layer of Bruch's membrane and the RPE. This material can appear similar to soft drusen histologically, with the exception that it is not mounded.

Knowledge of drusen composition, especially as it relates to phenotype, is scant. Wolter and Falls (1962) observed that drusen stain with oil red O, indicating the presence of neutral lipids in at least some drusen. Pauleikhoff, et al. (1992) used lipid-based histochemical staining approaches to show that different phenotypes of drusen contain either phospholipids or neutral lipids. These “hydrophilic” drusen were also bound by an anti-fibronectin antibody. Pauleikhoff et al. (1992) concluded that phospholipid-containing, but not neutral lipid-containing, drusen were anti-fibronectin antibody-reactive. Other investigators have not been able to reproduce the observation of an association of fibronectin with drusen (van der Schaft, et al., 1993; Mullins et al., 1999). These data suggest that drusen are either hydrophobic or hydrophilic, and that different drusen classes may indicate significantly different pathologies, suggesting the existence of different compositional classes of drusen, not solely based on morphology (i.e., hard and soft).

Farkas, et al. (1971b) analyzed drusen composition by enzymatic digestion, organic extraction, and histochemical staining methods for carbohydrates and other molecules. They concluded that drusen are comprised of sialomucins (glycoproteins with O-glycosidically-linked oligosaccharides) and cerebrosides and/or gangliosides.

Newsome et al. (1987) described labeling of soft drusen with antibodies directed against fibronectin, and to hard and soft drusen with antibodies directed against IgG and IgM. In addition, weak labeling of drusen with antibodies directed against beta amyloid (Loeffler, et al., 1995) and complement factors (C1q, C3c, C3d, and C4) (van der Schaft, et al., 1993), and more intense labeling with antibodies directed against ubiquitin (Loeffler and Mangini, 1997) and TIMP-3 (Fariss, et al., 1997), has been reported. Antibodies to other ECM molecules, including collagen types I, III, IV, and V, laminin, and heparan sulfate proteoglycan, have also been reported as being components of drusen in “diffuse, mottled or superficial laminar” patterns (Newsome, et al., 1987).

Discrepancies between the results of the immunohistochemical studies described above are likely due to disagreement upon a universal classification system for drusen, the use of dehydrated, paraffin-embedded tissues (which potentially resulting in the extraction of some drusen constituents) as opposed to frozen sections, and the use of antibodies directed against different epitopes of the same protein. Additionally, the use of tissues that are fixed or frozen within a short period after death reduces false negatives (due to post-mortem autolysis and loss of antigenicity) and false positives (due to post-mortem diffusion and loss of physiologic barriers).

Though the literature contains anecdotal reports about drusen composition, a comprehensive understanding of drusen biogenesis is lacking. At least twelve pathways for drusen genesis have been suggested in the literature (Duke-Elder and Dobree, 1967; Wolter and Falls, 1962; Ishibashi, et al., 1986a). These fall into two general categories based on whether drusen are derived from the RPE or the choroid. Theories related to the derivation of drusen from RPE cells include the concepts that: drusen result from secretion of abnormal material derived from RPE or photoreceptors (“deposition theories”—Muller, 1856; Ishibashi, et al., 1986; Young, 1987); transformation of degenerating RPE cells into drusen (“transformation theories”—Donders, 1854; Rones, 1937; Fine, 1981; El Baba, et al., 1986) or some combination of these pathways. Specifically, some investigators have concluded, based on ultrastructural data, that drusen are formed when the RPE expels its basal cytoplasm into Bruch's membrane (Ishibashi, et al., 1986a), possibly as a mechanism for removing damaged cytosol (Burns and Feeney Burns, 1980). However, very few convincing images of this process have been demonstrated. Others have postulated that drusen are formed by autolysis of the RPE, due to aberrant lysosomal enzyme activity (Farkas, et al., 1971a), although more recent enzyme histochemical studies have failed to demonstrate the presence of lysosomal enzymes in drusen (Feeney-Burns, et al., 1987). Other mechanisms, including lipoidal degeneration of the RPE (Fine, 1981) and a derivation from vascular sources (Friedman, et al., 1963) have also been postulated (summarized in Duke-Elder and Dobree, 1967). Farkas et al. (1971a) described the presence of numerous degenerating organelles in drusen, including what appeared to be lysosomes. Based on the observation that similar material was present on the RPE side of Bruch's membrane prior to drusen formation, they suggested that drusen constituents were derived from the RPE. However, lysosomal enzyme activity within drusen has not been verified (Feeney-Bums, et al., 1987). Burns and Feeney-Burns (1980) described the presence of “cytoplasmic debris” in small drusen, which they inferred was derived from the RPE. Feeney-Burns and Ellersieck (1985) later described a paucity of debris in Bruch's membrane directly beneath drusen, and suggested that drusen may result from an inability of the choroid to clear debris from sites of drusen deposition.

Ishibashi et al. (1986) observed cellular extensions of the RPE that protruded through the RPE basal lamina and into Bruch's membrane in eyes that were surgically enucleated for melanoma, suggesting that drusen possess, and may be derived from, RPE cell constituents. However, it should be noted that changes in RPE cytoskeletal organization and cell shape have been described in eyes with choroidal melanoma (Wallow an Tso, 1972; Fuchs, et al., 1991), making it difficult to draw conclusions about the derivation of drusen during normal senescence from these studies. Duvall et al. (1985) suggested a role for choroidal pericytes in keeping Bruch's membrane clear of debris. They suggested that dysfunction of pericytes leads to the formation of drusen, either by the accumulation of material from the choroid or by the failure to remove material deposited by the RPE. Penfold et al. (1986) have suggested a role for giant cells and mononuclear phagocytes in the pathology of the atrophic form of senile macular degeneration (see also Dastgheib and Green, 1994).

Burns and Feeney-Burns (1980) suggested that apoptosis, resulting in basal shedding of RPE cytosol, gives rise to drusen. Drusen-associated membranous profiles were inferred to be derived from the RPE, due to their localization between the RPE basal lamina and the inner collagenous zone of Bruch's membrane. While a number of investigators cite ultrastructural evidence for the derivation of drusen from RPE, the presence of melanin, lipofuscin or other RPE-derived organelles in drusen has not been reported.

It is clear that new diagnostics and therapeutics for drusen associated ocular diseases are needed. For example, there is currently no reliable means for diagnosing AMD. In addition, there is no available therapy that significantly slows the degenerative progression of AMD for the majority of patients. Current AMD treatment is limited to laser photocoagulation of the subretinal neovascular membranes that occur in 10-15% of affected patients. The latter may halt the progression of the disease but does not reverse the dysfunction, repair the damage, or improve vision.

### BRIEF SUMMARY OF THE INVENTION

In one aspect, the invention provides methods for diagnosing or identifying a predisposition to the development of a drusen associated ocular disorder in a subject. The methods comprise detecting the presence, activity or expression level of a drusen associated marker. The drusen associated ocular disorders that can be diagnosed with the methods include macular degeneration, North Carolina macular dystrophy, Sorsby's fundus dystrophy, Stargardt's disease, pattern dystrophy, Best disease, dominant drusen, and radial drusen. Specifically, the methods can be used for diagnosis of age-related macular degeneration.

In some of the methods, the drusen associated marker-to be detected is a phenotypic marker. The phenotypic marker can be RPE dysfunction and/or death, immune mediated events, dendritic cell activation, dendritic cell migration and differentiation, extrusion of the dendritic cell process into the sub RPE space, and the presence of geographic atrophy or disciform scars. In some methods, the drusen associated marker to be detected is a genotypic marker.

Other drusen associated markers that can be detected include markers involved in immune mediated events associated with drusen formation; markers involved in RPE dysfunction and/or death; markers expressed by choroidal and RPE cells; molecules associated with drusen; markers of drusen-associated dendritic cells; dendritic cell-associated accessory molecules that participate in T cell recognition; markers associated with dendritic cell expression; markers associated with dendritic cell proliferation; and markers associated with dendritic cell differentiation.

Some of the drusen associated markers to be detected with methods of the present invention are indicative of an immune mediated process at the RPE-Bruch's membrane-choroid interface. In some methods, the drusen associated marker can be an autoantibody. The autoantibody can be one directed against drusen, an autoantibody directed against RPE, an autoantibody directed against a B cell, an autoantibody directed against a T cell, an autoantibody directed against a macrophage, an autoantibody directed against a dendritic cell, an autoantibody directed against a systemic antigen, or an autoantibody directed against a neoantigen.

In one aspect, the invention provides methods for treating or preventing the development of a drusen associated ocular disorder in a subject. The methods comprise providing to the subject an effective amount of an agent which inhibits immune cell migration, proliferation, or differentiation. In some methods, the immune cell is a dendritic cell or a dendritic cell precursor. In other methods, the immune cell is a B cell or a T cell.

In some methods, the agent inhibits dendritic cell migration and extension of its process through Bruch's membrane and/or into the sub-retinal pigment epithelial. In some methods, the agent inhibits the initiation or maintenance of a cellular or humoral immune response. In some other methods, the agent disrupts antigen presentation and dendritic cell-T cell interaction.

Some of the methods are directed to inhibition of migration of the immune cell. In some of these methods, the agent employed is an agonist of a cytokine selected from the group consisting of GMCSF, TNIFα, and IL-1. Some methods are directed to inhibition of proliferation of the immune cell. An agent for such methods can be selected from the group consisting of antagonists of GMCSF, IL-4, IL-3, SCF, FLT-3, and TNFα. In still some other methods, the agent inhibits differentiation of the immune cell. Such an agent can be selected from the group consisting of IL-10, M-CSF, IL-6, and IL-4. Alternatively, it can be selected from the group consisting of antagonists TNF-α, IL-1, GM-CSF, IL-4, and IL-13.

The present invention also provides methods for inhibiting drusen formation or enhancing drusen resolution in a subject. Such methods comprise providing to the subject an effective amount of an agent which inhibits the gene expression or activity of one or more drusen associated molecules (DRAMs). In some methods, the DRAM is selected from the group consisting of apolipoprotein E, immunoglobulins, factor X, amyloid P component, complement C5, complement C5b-9 terminal complexes, fibrinogen, prothrombin, thrombospondin, and vitronectin.

The invention further provides methods for identifying an agent for treating or preventing drusen formation in a subject. Such methods entail administering to an agent to said subject in a non-toxic dosage and determining whether drusen formation is inhibited or drusen has resolved.

Other features and advantages of the invention will be apparent from the following Detailed Description and Claims.

### BRIEF DESCRIPTION OF THE DRAWINGS

FIG. 1 is a schematic representation of the retina and choroid, as seen in (A) histological section, and (B) retinal neurons shown diagrammatically. A, amacrine cells; B, bipolar cells; BM, Bruch's membrane; C, cone cells; CC, choriocapillaris; ELM, external limiting membrane; G, ganglion cells; GCL, ganglion cell layer; H, horizontal cells; ILM, inner limiting membrane; INL, internal nuclear layer; IPM, interphotoreceptor matrix; IS, inner segments of rods and cones; IPL, internal plexiform layer; NFL, nerve fiber layer; ONL, outer nuclear layer; OPL, outer plexiform layer; OS, outer segments of rods and cones; PE, pigment epithelium; PRL, photoreceptor layer; PT, photorecptor cell terminals; R, rod cells; ST, stroma vascularis of choroid.

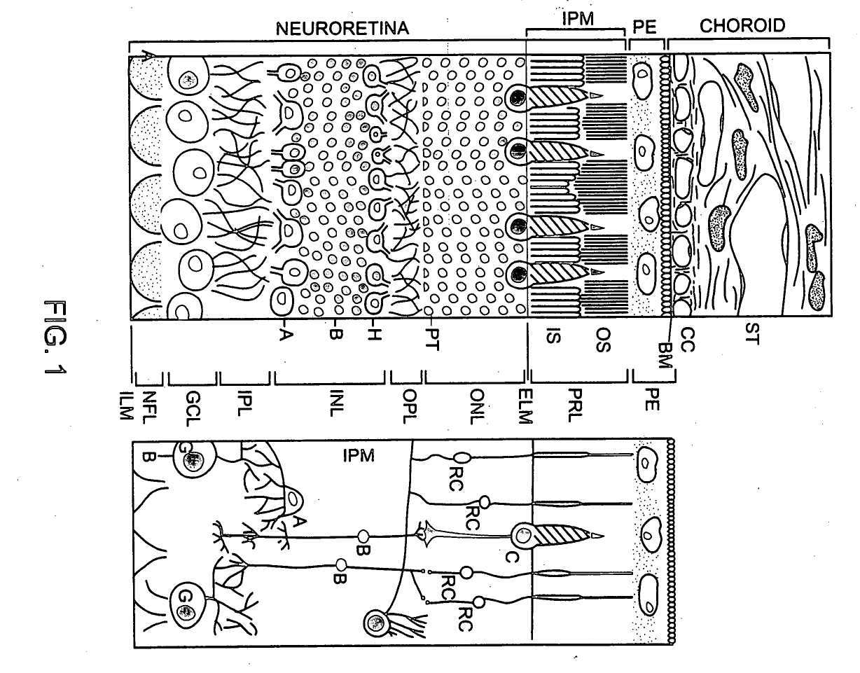

### DETAILED DESCRIPTION OF THE INVENTION

### I. DEFINITIONS

The meaning of certain terms and phrases as used in the following detailed description and claims are defined as follows:

The term “agonist”, as used herein, is meant to refer to an agent that enhances or upregulates (e.g., potentiates or supplements) the production or activity of a gene product. An agonist can also be a compound which increases the interaction of a gene product, molecule or cell with another gene product, molecule or cell, e.g., of a gene product with another homologous or heterologous gene product, or of a gene product with its receptor. A preferred agonist is a compound which enhances or increases binding or activation of a transcription factor to an upstream region of a gene and thereby activates the gene. Any agent that activates gene expression, e.g., by increasing RNA or protein synthesis or decreasing RNA or protein turnover, or gene product activity may be an agonist whether the agent acts directly on the gene or gene product or acts indirectly, e.g., upstream in the gene regulation pathway. Agonists may be RNAs, peptides, antibodies and small molecules, or a combination thereof.

The term “animal model”, as used herein, includes transgenic animals, naturally occurring animals with genetic mutations and non-transgenic animals that have been treated with one or more agents, or combinations thereof (e.g., a skid mouse), any of which may serve as experimental models for a disease, e.g., macular degeneration. For example, a transgenic mouse may be a mouse in which a gene is knocked out or in which a gene is overexpressed.

The term “antagonist” as used herein is meant to refer to an agent that downregulates (e.g., suppresses or inhibits) the production or activity of a gene product. Such an antagonist can be an agent which inhibits or decreases the interaction between a gene product, molecule or cell and another gene product, molecule or cell. A preferred antagonist is a compound which inhibits or decreases binding or activation of a transcription factor to an upstream region of a gene and thereby blocks activation of the gene. Any agent that inhibits gene expression or gene product activity may be an antagonist whether the agent acts directly on the gene or gene product or acts indirectly, e.g., upstream in the gene regulation pathway. An antagonist can also be a compound that downregulates expression of a gene or which reduces the amount of gene product present, e.g., by decreasing RNA or protein synthesis or increasing RNA or protein turnover. Antagonists may be RNAs, peptides, antibodies and small molecules, or a combination thereof.

The term “associate” or “interact” as used herein is meant to include detectable relationships or associations (e.g., biochemical interactions) between molecules, such as interaction between protein-protein, protein-nucleic acid, nucleic acid-nucleic acid, protein-carbohydrate, carbohydrate-carbohydrate, protein-lipid, lipid-lipid, etc., and protein-small molecule or nucleic acid-small molecule in nature.

“Bruch's Membrane” is a trilaminar extracellular matrix complex that lies between the retinal RPE and the-primary capillary bed of the choroid, the choriocapillaris. Bruch's membrane is comprised of two collagen layers, referred to as the inner and outer collagenous layers, that flank a central domain comprised largely of elastin. The strategic location of Bruch's membrane between the retina and its primary source of nutrition, the choroidal vasculature, is essential for normal retinal function (Marshall et al., 1998; Guymer and Bird, 1998). Imunohistochemical studies have documented the presence of collagen types I, III, IV, V and VI within Bruch's membrane proper (Das et al., 1990; Marshall et al., 1992). Type VI is associated specifically with the elastic lamina, types IV and V with the basal laminae of the choriocapillaris and RPE, and types I and III with the inner and outer collagenous layers. The presence of collagen types I, III, IV and V in these tissues has also been confirmed biochemically.

The term “choroid” refers to the highly vascularized tissue lying between the sclera and retinal pigment epithelium of the eye. This tissue is comprised of numerous pericytes, melanocytes, fibroblasts, myofibroblasts and transitional leukocytes. “Bruch's membrane, a trilamellar extracellular matrix comprised of inner and outer collagenous layers and an elastic lamina, is a component of the choroid. It is positioned between the basal lamina of the RPE and the choriocapillaris. The remaining extracellular matrix of the choroid is comprised of a variety of extracellular matrix constituents that are loosely organized.

The term “dendritic cell” or “DC” as used herein refers to hematopoietic cells characterized by their unusual dendritic morphology, their potent antigen-presenting capability and their lack of lineage-specific markers such as CD3, CD19, CD16, CD14, which distinguishes them respectively from T cells, B cells, NK cells, and monocytes. Currently there are at least two ontogenic pathways for dendritic cell development: those that derive from myeloid-committed hematopoietic precursors and those that derive from lymphoid-committed hematopoietic precursors. Myeloid-committed precursors which give rise to granulocytes and monocytes can also differentiate into Langerhans cells of the skin and myeloid related dendritic cells in the secondary lymphoid tissue. There may also be a class of lymphoid-derived dendritic cells (See Lotze, M. T. and Thomson, A. W. (Eds.) (1999) “Dendritic Cells”, Academic Press, San Diego, Calif., for a number of reviews on dendritic cells, the teachings of which are incorporated herein by reference).

The term “dendritic cell precursor” or “DC precursor” as used herein refers to cell types from which a dendritic cell is derived upon differentiation and maturation. A dendritic cell precursor may be a bone marrow stem cell, a lymphiod cell lineage-committed cell or a myeloid cell lineage-committed cell from which a dendritic cell may develop after exposure to-certain factors. For exarnple,.DC precursors-of the myeloid lineage can be induced to differentiate into DCs by treatment with GM-CSF.

The term “drusen” as used herein encompasses a number of phenotypes, all of which develop, between the inner collagenous layer of Bruch's membrane and the RPE basal lamina. Hard drusen are small distinct deposits comprised of homogeneous eosinophilic material and are usually round or hemispherical, without sloped borders. Soft drusen are larger, usually not homogeneous, and typically contain inclusions and spherical profiles. Some drusen may be calcified. The term “diffuse drusen,” or “basal linear deposit,” is used to describe amorphous material which forms a layer between the inner collagenous layer of Bruch's membrane and the retinal pigment epithelium (RPE). This material can appear similar to soft drusen histologically, with the exception that it is not mounded.

The term “drusen-associated marker” (DRAM) refers to a phenotype or genotype that is involved or associated with the development of drusen formation and ultimately the development of a drusen associated ocular disease or disorder. Examples of phenotypic markers include: RPE dysfunction and/or death, immune mediated events, dendritic cell activation, migration, differentiation and extrusion of the DC process into the sub RPE space (e.g. by detecting the presence or level of a dendritic cell marker such as CD68, CD1a and S100), and the presence of geographic atrophy or disciform scars.

Examples of genotypic DRAMs include mutant genes and the encoded mutant polypeptide, abnormal expression of proteins, abnormal levels of expressed gene products (including mRNA or protein levels that are upregulated or downregulated), and/or a distinct pattern of differential gene expression in drusen forming ocular tissues. Markers expressed by dysfunctional and/or dying RPE cells include HLA-DR, CD68, vitronectin, apolipoprotein E, clusterin, and S-100. Markers expressed by choroidal and RPE cells in AMD include heat shock protein 70, death protein, proteasome, Cu/Zn superoxide dismutase, cathepsins, and death adaptor protein RAIDD. Markers involved in immune mediated events are associated with drusen formation include: autoantibodies, leukocytes, dendritic cells, myofibroblasts, type VI collagen, chemokines, and cytokines.

Autoantibody markers can be autoantibodies directed against drusen, RPE, retina components, or other local antigens (e.g., antigens of ECM). Autoantibodies against autoantigens such as vitronectin, β crystallin, calreticulin, serotransferrin, keratin, pyruvate carboxylase, C1, and villin 2 are examples of such markers. They can also be directed to newly exposed antigens or neoantigens, other local, and systemic antigens. Neoantigens are antigens resulting from modification and/or crosslinking of existing molecules by various processes such as oxidation. Examples of neoantigens include neoantigens associated with oxidized LDL in atherosclerosis (Reaven et al., Adv Exp Med Biol, 366:113-28, 1994; Kita et al., Ann N Y Acad Sci, 902:95-100, 2000), or oxidation-derived complex in other diseases (Ratnoffet al., Am J Reprod Immunol, 34:72-9 1995; and Debrock et al., FEBS Lett, 376:243-6, 1995). Autoantibodies against autoantigens from other tissues (systemic antigens) are indicators of a systemic nature of the underlying disease or disorder.

Other DRAMs are molecules that have been shown to be associated with drusen (see Table 2). These markers include immunoglobulins, amyloid A, amyloid P component, HLA-DR, fibrinogen, Factor X, prothrombin, complements 3, 5, 9, and 5b-9, C- reactive protein (CRP) apolipoprotein A, apolipoprotein E, antichymotrypsin, β2 microglobulin, thrombospondin, and vitronectin. Markers of drusen associated dendritic cells include: CD1a, CD4, CD14, CD31 (PECAM-1), CD45, CD64/1 (FcR), CD68, CD83, CD86 and HLA-DR, particular preferred dendritic cell markers include CD1a, CD 14, CD45, CD68, CD83 and HLA-DR. Important dendritic cell-associated accessory molecules that participate in T cell recognition include ICAM-1, LFAI, LFA3, and B7, IL-1, IL-6, IL-12, TNF-alpha, GM-CSF and heat shock proteins. Markers associated with dendritic cell expression include: colony stimulating factor, TNFα, and IL-I. Markers associated with dendritic cell proliferation include: GM-CSF, IL-4, IL-3, SCF, FLT-3 and TNFα. Markers associated with dendritic cell differentiation include IL-10, M-CSF, IL-6 and IL-4.

The term “drusen-associated ocular disorder” as used herein refers to any disease or disorder which involves drusen formation. For example, in macular degenerations, the accumulation of drusen creates a physical barrier that appears to impede normal metabolite and waste diffusion between the choriocapillaris and the retina. As a result, the diffusion of oxygen, glucose, and other nutritive or regulatory serum-associated molecules required to maintain the health of the retina and RPE are inhibited.

A “drusen-associated molecule” or “DRAM” as used herein refers to any protein, carbohydrate, glycoconjugate (e.g., glycoprotein or glycolipid), other lipid, nucleic acid or other molecule which is found in association with, or interacting with, a drusen deposit. DRAMS may include cellular fractions or organelles that are not normally found deposited in, or in association with, a tissue unless it is affected by drusen or which is not present in drusen-affected and normal tissue in equivalent amounts.

The term “extracellular matrix” (“ECM”) refers to, e.g., the collagens, proteoglycans, non-collagenous glycoproteins and elastins that surround cells and provide structural and fimctional support for cells as well as maintain various functions of cells, such as cell adhesion, proliferation, differentiation and protein synthesis. A skilled artisan will appreciate that the precise composition and physical properties of ECM, as well as its function, vary between various cell types, between various tissues, and between various organs.

The term “inhibit” as used herein means to prevent or prohibit and is intended to include total inhibition, partial inhibition, reduction or decrease.

The term “macular degeneration” refers to any of a number of conditions in which the retinal macula degenerates or becomes dysfunctional, e.g., as a consequence of decreased growth of cells of the macula, increased death or rearrangement of the cells of the macula (e.g., RPE cells), loss of normal biological function, or a combination of these events. Macular degeneration results in the loss of integrity of the histoarchitecture of the cells of the normal macula and/or the loss of ftnction of the cells of the macula. The term also encompasses extramacular changes that occur prior to, or following dysfunction and/or degeneration of the macula. Any condition which alters or damages the integrity or function of the macula (e.g., damage to the RPE or Bruch's membrane) may be considered to fall within the definition of macular degeneration. Other examples of diseases in which cellular degeneration has been implicated include retinal detachment, chorioretinal degenerations, retinal degenerations, photoreceptor degenerations, RPE degenerations, mucopolysaccharidoses, rod-cone dystrophies, cone-rod dystrophies and cone degenerations.

The terms “modulation”, “alteration”, “modulate ”, or “alter ” are used interchangeably herein to refer to both upregulation (i.e., activation or stimulation (e.g., by agonizing or potentiating) and downregulation (i.e., inhibition or suppression (e.g., by antagonizing, decreasing or inhibiting)) of an activity. For example, the activity that is modulated may be gene expression or may be the growth, proliferation, migration or differentiation of dendritic cells. “Modulates” or “alters” is intended to describe both the upregulation or downregulation of a process, since, as is well known to a skilled artisan, a process which is upregulated by a certain stimulant may be inhibited by an antagonist to that stimulant. Conversely, a process that is downregulated by a certain stimulant may be inhibited by an antagonist to that stimulant. Thus, e.g., the identification of an agent that induces a cellular response modulates or alters cellular behavior in an inductive manner and it is inherently understood that the response may be modulated in an inhibitory manner by an inhibitor of that agent (e.g., by an antibody or antisense RNA, as is well understood and described in the art).

The term “nucleic acid” as used herein refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long. A “polymorphic gene” refers to a gene having at least one polymorphic region.

The terms “protein”, “polypeptide” and “peptide” are used interchangeably herein when referring to a gene product comprising amino acids. The term “recombinant protein” refers to a polypeptide of the present invention which is produced by recombinant DNA techniques, wherein generally DNA encoding a polypeptide is inserted into a suitable expression vector which is in turn used to transform a host cell to produce the heterologous protein. Likewise the term “recombinant nucleic acid” or “recombinant DNA” refers to a nucleic acid or DNA of the present invention which is produced by recombinant DNA techniques, wherein generally DNA encoding a polypeptide is inserted into a suitable expression vector which is in turn used to transform a host cell to produce the heterologous protein. Moreover, the phrase “derived from”, with respect to a recombinant gene, is meant to include within the meaning of “recombinant protein” those proteins having an amino acid sequence of a native polypeptide, or an amino acid sequence similar thereto which is generated by mutations including substitutions and deletions (including truncation) of a naturally occurring form of the polypeptide.

The term “retinal pigment epithelium” or “RPE” refers to the cuboidal epithelial monolayer that is situated between the neural retina and choroid. The RPE derives developmentally from, and is indeed contiguous with, the same neuroectodermal layer as the neural retina. The RPE possesses numerous large pigment granules (melanosomes) which participate in the prevention of light scattering. In addition, the RPE plays a critical role in the maintenance of photoreceptor cell viability and function by the phagocytosis and removal of photoreceptor outer segment disks, the processing and secretion of various molecules necessary for photoreceptor function and viability (such as vitamin A derivatives and growth factors), the regulation of macromolecular traffic between the retina and choroid, and the mediation of retinal adhesion.

The term “small molecule” as used herein, is meant to refer to a composition which has a molecular weight of less than about 5 kD and most preferably less than about 4 kD. Small molecules can be nucleic acids, peptides, polypeptides, peptidomimetics, carbohydrates, lipids (e.g., glycolipids and pig-tail lipids) or other organic (carbon containing) or inorganic molecules. Many pharmaceutical companies have extensive libraries of chemical and/or biological mixtures, often fungal, bacterial, or algal extracts, which can be screened with any of the assays of the invention to identify therapeutic compounds.

The term “therapeutic” as used herein refers to an agonist or antagonist of the bioactivity of a drusen associated marker. Preferred therapeutics reduce or inhibit RPE cell death, factors involved in the inflammatory response, factors involved in fibroblast proliferation and migration resulting in dendritic cell activation, migration or differentiation into drusen. Examples of therapeutics of the present invention include antiinflammatory agents (e.g. anti CD- 18 antibody), protease inhibitors, inhibitors of elastolytic MMPs (e.g. the hydroxamate based RS3 12908, batimastat, antibiotics (e.g. doxycycline), tetracycline), inhibitors of prostaglandin synthesis and beta-blockers (e.g. propanalol).

The term “transcriptional regulatory sequence” is a generic term used throughout the specification to refer to DNA sequences, such as initiation signals, enhancers, and promoters, which induce or control transcription of protein coding sequences with which they are operably linked.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA.

As used herein, the term “transgene” means a nucleic acid sequence (encoding, e.g., one of the polypeptides of the invention, or an antisense transcript thereto) which has been introduced into a cell. A transgene could be partly or entirely heterologous, i.e., foreign, to the transgenic animal or cell into which it is introduced, or can be homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout or may result in over expression). A transgene can also be present in a cell in the form of an episome. A transgene can include one or more transcriptional regulatory sequences and any other nucleic acid, such as 5′ UTR sequences, 3′ UTR sequences, or introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to fail to express a specific normal gene product, to express a recombinant form of one or more DRAM polypeptides, e.g., either agonistic or antagonistic forms, or molecules that regulate the biosynthesis, accumulation or resorption of DRAMs or dendritic cells. Transgenic knockouts may, for example, be produced which cause alterations in dendritic cell behavior (e.g., cell growth, proliferation, migration, differentiation or gene expression). For example, mice whose Rel-B, transforming growth factor β1 (TGF-β1) or Ikaros genes are disrupted lack dendritic cells from various cell lineages (see Caux, C. et al., 1999). However, transgenic animals in which the recombinant DCRM or DRAM gene is silent are also contemplated, as for example, the FLP or CRE recombinase dependent constructs. Moreover, “transgenic animal” also includes those recombinant animals in which gene disruption is caused by human intervention, including both recombination and antisense techniques.

The term “treating” as used herein is intended to encompass curing as well as ameliorating at least one symptom of the condition or disease.

The terms “vector,” “cloning vector,” or “replicative cloning vector,” are interchangeable as used herein, and refer to a nucleic acid molecule, which is capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors.” The term “expression system” as used herein refers to an expression vector under conditions whereby an mRNA may be transcribed and/or an MRNA may be translated into protein. The expression system may be an in vitro expression system which is commercially available or readily made according to art known techniques, or may be an in vivo expression system, such as a eukaryotic or prokaryotic cell containing the expression vector. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as a plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

### II. OVERVIEW

The invention is based, at least in part, on the discovery of the etiology of AMD and other drusen-associated ocular disorders, essentially as described below.

A. Drusen Biogenesis

Outlined herein is a unifying model of drusen biogenesis. This model is put forth with the understanding that numerous AMD genotypes can exist. Thus, only some aspects of the discussed mechanisms may be involved in any given AMD genotype. Importantly, the model is based upon novel data generated by the inventors which indicate that dendritic cells are associated with drusen biogenesis. This observation invokes, for the first time, the potential for a direct and integral role of immune cell-mediated processes in drusen biogenesis.

The presence of dendritic cells in inflammatory lesions is well-recognized. It is clear that dendritic cells must be recruited, activated, and migrate to, sites of inflammation, rather than passively migrating to these sites. Dendritic cells are typically recruited to sites of tissue damage by various chemoattractants, heat shock proteins, DNA fragments, and others. Choroidal dendritic cell processes are associated with the smallest of drusen, and are often observed in the sub-RPE space in association with whole, or portions of, RPE cells that have been shunted into Bruch's membrane, prior to the time that drusen, per se, are detectable. Based on these observations, proposed herein is a mechanism in which choroidal dendritic cells are activated and recruited by locally damaged and/or sublethally injured RPE cells. This idea is consistent with recent data showing that dendritic cells, and thus the innate immune system, can be activated by microenvironmental tissue damage. In this state, these cells extend a cellular process through Bruch's membrane in order to gain access to the site of tissue damage. In this role, choroidal dendritic cells may thus serve as sentinel receptors with the capacity to respond to local cell injury, and ultimately provide for the overall integration of immune-mediated processes that determine the outcome of the overall response.

In this model, the injured RPE and/or other local cells can serve as a source of soluble cytokines or other stimulatory factors that initiate dendritic cell recruitment and activation. The data presented herein clearly supports accelerated RPE cell death in eyes derived from donors with AMD, as compared to age-matched controls. Based on available information from other systems, and upon previous suggestions pertaining to the etiology of AMD, RPE cell death might occur by several mechanisms, including ischemia, necrosis, gene-mediated injury, Bruch's membrane-induced dysfunction, oxidative injury from light or systemic factors (e.g. smoking-generated compounds), lipofuscin accumulation, or autoimmune phenomena, to list a few. Based on data disclosed herein, it is likely that RPE cell death would most likely have to be due to necrosis, rather than to apoptosis, since cells undergoing apoptotic cell death are not known to be capable of recruiting dendritic cells. Indeed, the data provides compelling evidence for an absence of apoptotic RPE cell death in human donor eyes. Further, additional data disclosed herein indicate that death or dysfunction of choriocapillaris-associated cells can be involved in the process of dendritic cell recruitment and activation.

Several known pathways can initiate receptor-ligand interactions between dendritic cell precursors and injured tissue. These include cytokines such as IL-1, IL-6, IL-12, TNF-alpha, and GM-CSF, heat shock proteins, altered expression of cell surface proteins and DNA in the presence of free radicals. The novel observation of clonal expression of HLA-DR, CD68, vitronectin, S-100, clusterin, and apolipoprotein E by RPE cells in eyes from donors with drusen may be particularly significant in this respect. Furthermore, up-regulation of various cell death- and immune-associated molecules by the RPE/choroid in eyes with developing drusen and AMD have been identified using differential display and gene array analyses. In addition, there is evidence that free radicals, which are known to be present in high concentrations at the RPE-retina-choroid interface, might be immunostimulatory. There is also data suggesting that ceroid (a potential component of lipofuscin) derived from necrotic cells may serve as an antigen in the generation of certain autoimmune diseases. This could explain the general contention that oxidative stress and/or lipofuscin may lead to RPE dysfunction and the development of AMD (Mainster, M. A., Light and macular degeneration: a biophysical and clinical perspective. Eye, 1987, 1 (Pt 2): p.304-10).

Once inside the pre-lesion or lesion (a.k.a. the drusen, or drusen precursor site), dendritic cells might then contribute to the chronicity (induced chronic inflammatory lesions) of AMD by any number of mechanisms, including immune complex formation, complement activation, and/or in situ activation of choroidal T-cells, other phagocytic cells, and matrix proteolysis. The presence of numerous immune-associated constituents in drusen, including immunoglobulins, complement proteins, and some acute phase proteins, could be explained by such an event. One might predict that the dendritic cell response would be down-regulated once the local tissue damage has been repaired, thus restoring tolerance. This type of self-limiting control is typically accomplished in other systems via turnover of dendritic cells; the influx of new dendritic cell precursors and the concomitant reduction in the influx of mature dendritic cells into the lymph nodes is typically sufficient to shift the balance back to tolerance. In other cases, natural killer cells recognize mature dendritic cells as targets, providing a negative feedback effect on antigen presentation, forcing the system into tolerance. However, in the case of AMD, a state of chronic inflammation can persist for many years. In this scenario, cyclical events of RPE cell death may occur over a period of many years that do not allow the system to return to tolerance. In one example, this might occur as a result of genetic preprogramming, as in the case of a RPE gene mutation. In another example, local activation of complement and HLA-DR expression by RPE cells, initiated by dendritic cells recruited to the sub-RPE region, might lead to clonal RPE cell death, thereby maintaining a state of chronic inflammation. A negative outcome of this entire process may be that Bruch's membrane and the surrounding extracellular matrix may be degraded, angiogenic factors may be generated, resulting in opportunistic neovascularization of the sub-RPE and subretinal spaces. Although there is little information in the literature concerning matrix-degrading enzyme expression by dendritic cells, MT-1-MMP expression within drusen cores has been observed, suggesting a possible mechanism for DC-mediated matrix breakdown.

The notion that dendritic cells may be activated by local tissue injury might also initiate an autoimmune response to retinal, RPE, and/or other local antigens that are uncovered during tissue damage. The availability and amount of debris/antigen will most likely determine which ensuing pathway is involved. Such autoimmune responses have been documented as a consequence of ischemia or injury to the heart. The inventors have recently identified autoantibodies in the sera of individuals with AMID that are directed against retinal and RPE proteins of 35 kDa-and 53 kDa. This might occur as a consequence of aberrant delayed-type hypersensitivity responses, perhaps explaining the presence of serum autoantibodies in at least some AMD patients. It is also conceivable that the groundwork for this autoimmune process is primed earlier in life by necrosis of RPE cells. Indeed, this would explain the inventor's observation of a wave of peripheral RPE cell dropout in the second and third decades of life. Alternatively, neoantigens could be formed in the vicinity of drusen via any number of established mechanisms, similar to that implicated in atherosclerosis (Reaven et al., Adv Exp Med Biol, 366:113-28, 1994; and Kita et al., Ann N Y Acad Sci, 902:95-100, 2000). It is also possible that the autoimmune response is initiated in extracellular tissues and that this response damages ocular cells and tissues secondarily.

In the model presented herein, the RPE injury or other local cell and dendritic cell events are followed by the continued deposition of drusen-associated constituents. Early DRAM-matrix complexes, such as immune complexes, or other local ligands might serve as “nucleation sites” for the deposition of additional self-aggregating proteins and/or lipids. These constituents could be derived from either the plasma and/or local cellular sources. Based on the knowledge that many DRAMs are circulating plasma proteins, it is plausible that some DRAMs pass out of choroidal vessels and into the extracellular space adjacent to the RPE where they bind to one or more ligands associated with Bruch's membrane in the aging eye. These ligands could be basement membrane components, plasma membrane receptors, secretory products derived from RPE or choroidal cells, or byproducts of cellular autolysis. As reported herein, a number of drusen-associated molecules, including apolipoprotein E, vitronectin, fibrinogen, C reactive protein, and transthyretin, have been synthesized by the RPE and/or retina. Although unexpected, these data support the concept that some DRAMs may be synthesized and secreted locally. It remains to be determined conclusively whether up- or down-regulation of DRAM synthesis by local cells correlates with drusen deposition and/or AMD, although gene array analyses provide support for upregulated synthesis of a number of DRAMs, including immunoglobulins, by the RPE and choroid of AMD donors. As these abnormal drusen deposits increase in size they displace the RPE monolayer and are recognized clinically as drusen.

B. Role of RPE in Drusen Biogenesis

As described herein, Applicants have discovered that retinal pigment epithelial cell (RPE) dysfunction and death is certainly associated with the development of drusen and, by extension, in the etiology of drusen-associated ocular diseases.

First morphometric analyses of a Comprehensive Donor Database repository comprised of 168 donors, aged between 0 and 101, with and without a clinically documented history of drusen and AMD, provide strong evidence that the rate of RPE cell death in individuals with drusen and AMD is significantly higher than in age-matched controls. RPE cell loss in normal individuals occurs at a rate of between 10% and 15% over nine decades, in contrast to a rate between 30% and 40% in individuals with AMD and drusen. Significantly, it appears that the majority of RPE cell death likely occurs by a process of necrosis, rather than apoptosis. These observations are based on employment of the TUNEL assay, an absence of apoptosis-associated gene expression in gene array analyses and electron microscopic observation.

Second, fragments of RPE cells (identified on the basis of morphologically detectable lipofuscin and pigment granules), can be detected within drusen at both the light and electron microscopic levels of resolution, demonstrating that they contribute to drusen volume and formation.

Third, drusen-associated dendritic cell processes (as described in detail elsewhere herein) are often observed in association with these early stages of RPE fragmentation and “blebbing”, suggesting that the stimulus for dendritic cell recruitment lies at the level of RPE cells.

Fourth, RPE cells associated with the smallest of drusen (and regions presumed to be drusen precursors) are often characterized by focal expression of molecules not normally associated with these cells. These molecules include HLA-DR, CD68, vitronectin, apolipoprotein E, and perhaps clusterin and S-100. Although it is highly unusual for non-immunocompetent cells to express HLA-DR, this protein is typically expressed by cells early in immune reactions. Indeed, its expression by RPE cells may be a marker of RPE cell dysfunction and is likely to be involved in recognition of dysfunction and/or damaged RPE by other cells. Alternatively, the expression of HLA-DR might be a secondary phenomenon related to the presence of dendritic cells.

Fifth, gene array analyses of RPE/choroid preparations from AMD and control donors indicate upregulation of a number of cell death associated molecules in AMD. These include, but are not limited to, death protein, heat shock protein 70, proteasome, Cu/Zn superoxide dismutase, cathepsins and death adaptor protein RAIDD.

It is unclear if drusen (or other abnormal changes in the extracellular environment that is Bruch's membrane) are a cause, or a consequence of RPE dysftmction. An accumulation of drusen could cause local interference with the exchange of metabolites and waste products between the choriocapillaris.and an otherwise normal RPE, leading to RPE dysfimction and death. On the other hand, drusen may be a consequence of aberrant RPE gene expression, although the precise biological events that ultimately lead to RPE dysfunction are equally unclear. Suggestions range from gene mutations to oxidative insults to lipofuscin accumulation, to programmed cell death. Whatever the progression of pathological events, localized RPE degeneration leads to a concomitant degeneration of the underlying photoreceptor cells, which in turn, result in the formation of numerous scotomas corresponding in size and in number to the distribution of macular drusen.

C. Immune-Mediated Processes and Drusen Biogenesis

Data from a variety of studies collectively suggest that immune-mediated events may participate in the development and/or progression of AMD. Autoantibodies have been detected in the sera of AMD patients (Guerne, D., et al., Antiretinal antibodies in serum of patients with age-related macular degeneration. Ophthalmology, 1991. 98: p. 602-7; Penfold, P., et al., Autoantibodies to retinal astrocytes associated with age-related macular degeneration. Graefe's Arch. Clin. Exp. Ophthalmol., 1990. 228: p. 270-4.). Some of these are directed against drusen, RPE and retina components based on immunohistochemical and Western analyses. Accumulations of giant multinucleated cells (Penfold, P., M. Killingsworth, and S. Sarks, Senile macular degeneration. The involvement of giant cells in atrophy of the retinal pigment epithelium. Investigative Ophthalmology & Visual Science, 1986. 27: p. 364-71; Dastgheib, K. and W. Green, Granulomatous reaction to Bruch's membrane in age-related macular degeneration. Archives of Ophthalmology, 1994. 112: p. 813-818); Penfold, P.L., et al., Modulation of major histocompatibility complex class II expression in retinas with age-related macular degeneration. Investigative Ophthalmology & Visual Science, 1997. 38(10): p. 2125-33.) and other leukocytes (Penfold, P., M. Killingsworth, and S. Sarks, Senile macular degeneration: the involvement of immunocompetent cells. Graefe's Archives for Clinical and Experimental Ophthalmology, 1985. 223:p.69-76);Killingsworth, M., J. Sarks, and S. Sarks, Macrophages related to Bruch's membrane in age-related macular degeneration. Eye, 1990. 4: p. 613-621) in the choroid of donors with AMD have been described and HLA-DR immunoreactivity of retinal microglia increases in AMD.

Exhaustive immunohistochemical analyses of drusen composition have revealed a distinct array of molecules (including immunoglobulins, amyloid A, amyloid P component, C5 and C5b-9 terminal complexes, HLA-DR, fibrinogen, Factor X, and prothrombin) that are common to all phenotypes of hard and soft drusen. Surprisingly, additional studies have documented that a number of these constituents (many of which have been thought to be synthesized primarily in the liver) are synthesized locally by RPE, retinal, and/or choroidal cells. These include complements 3, 5 and 9, complement reactive protein (CRP), immunoglobulin lambda and kappa light chains, Factor X, HLA-DR, apolipoprotein A, apolipoprotein E, amyloid A, vitronectin and others.

Interestingly, a number of these drusen-associated constituents (DRAMs) are participants in humoral and cellular immune processes. Moreover, it is indeed difficult to ignore the presence of some of these molecules, including terminal complement complex, immunoglobulin, and MHC class II antigens, in drusen. For example, C5b-9 complex is associated with specific immune processes, often involving cell death. Thus, the presence of C5b-9 in drusen and the expression of complement receptor genes by RPE and choroidal cells, including HCR1, HCR2, clusterin, vitronectin, and gp330/megalin brings to question the role of complement-mediated RPE cell death in drusen biogenesis and the etiology drusen-associated ocular disorders. Data from differential gene expression analyses indicate a significant up-regulation of a number of immune system-associated molecules (including Ig mu, lambda, J, and kappa chains) in the RPE/choroid of AMD donors, as compared to age-matched controls. Taken together, these data suggest that immune-related processes may be important in drusen development and the etiology AMD.

D. Dendritic Cells and Drusen Biogenesis

Dendritic cells are found in primary lymphoid organs and most non-lymphoid tissues and organs (Ibrahim, M., B. Chain, and D. Katz, The injured cell: the role of the dendritic cell system as a sentinel receptor pathway. Immunology Today, 1995. 16: p. 181-6; Matyszak, M. and V. Perry, The potential role of dendritic cells in immune-mediated inflammatory diseases in the central nervous system. Neuroscience, 1996. 74: p. 599-608; Matyszak, M. and V. Perry, Dendritic cells in inflammatory responses in the CNS, in Dendritic cells in fundamental and clinical immunology, Ricciardi-Castagnoli, Editor. 1997, Plenum Press: New York), with the possible exception of the central nervous system. Precursor dendritic cells reside within non-lymphoid tissues. Dendritic cells are powerful antigen-presenting cells that contribute to the pathogenesis of immune-mediated responses in a number of ways, including the primary activation of T lymphocytes, various secondary responses, and the induction of autoimmune responses. Antigen presentation is important in the induction of conventional immune responses, as well as in the induction and maintenance of tolerance. It has-been-proposed that dendritic-cells may provide an essential link between the innate and adaptive immune systems, actively participating in determining the outcome of the immune response. For example, data from recent investigations suggest that dendritic cells, and hence the innate immune system, can be activated by local, microenvironmental tissue damage. In this role, dendritic cells provide a sentinel receptor system that responds to local tissue injury and provides an integrative mechanism that determines the outcome of the immune response.

After acquiring an antigen, dendritic cells typically (but not always) migrate out of the tissue, into the blood, through the afferent lymphatics, and into the T cell-rich regions of the local lymphoid organs. Important dendritic cell-associated accessory molecules that participate in T cell recognition include ICAM-1, LFA1, LFA3, and B7, whereas T cell counter receptors include LFA1, CD2, and CD28. Binding of the B7 ligand to its counter receptor CD28 is especially important in stimulating the synthesis and secretion of IL-2 by T cells.

The results of studies described herein provide additional strong support for the involvement of immune-related processes in drusen biogenesis. Most notably, a novel and specific association has been noted between a subpopulation of choroidal cells and drusen. Ultrastructurally, processes of morphologically distinct choroidal cells are observed to breach Bruch's membrane and to terminate as bulbous, vesicle-filled cores within the centers of drusen. An association of specific cluster differentiation (CD) antigen and MHC class II markers indicates that these cells are certainly of monocytic origin, and are most likely dendritic cells. Specific marker molecules, including CD1a, CD4, CD14, CD68, CD83, CD86, and CD45, react with drusen-associated dendritic cells, suggesting that these cells belong to the DC1 lineage believed to participate in the induction of immunity. Additional immunocytochemical analyses document an intimate association of PECAM, MMP14, ubiquitin, and possibly FGF and HLA with drusen-associated dendritic cell cores.

Ongoing morphometric studies suggest that 40% of drusen in any given eye contain these structures and that at least 70% of donors with drusen possess at least one drusen core. Similar numbers have been obtained using different markers. Drusen cores are observed in all drusen phenotypes and are present in both macular and extramacular drusen. They may be more prevalent in drusen possessing a height-width ratio of less than 0.5.

E. Similar Etiology Between Drusen-Associated Ocular Disorders and Other Age-Related Diseases

Since drusen share a number of molecular constituents in common with abnormal deposits associated with a variety of other age-related diseases, drusen may represent an ocular manifestation of amyloidosis, elastosis, dense deposit disease, and/or atherosclerosis. Although modulated by different genes and/or environmental influences, all these diseases give rise to similar, yet distinguishable, pathological phenotypes by triggering a similar set of biological responses that include inflammation, coagulation, and activation of the immune system. Thus, the invention provides a valuable recognition of these similarities but also provides a method for diagnosing and treating drusen specifically, as compared to other age-related diseases which manifest themselves in deposits or plaques.

TABLE 1
Compositional Comparison of Extracellular Disease Plaques

| | | | Athero- | Dense | |
|---|---|---|---|---|---|
| | Elastosis | Amyloidosis | sclerosis | Deposits | Drusen |
| Vn | + | + | + | + | + |
| SAP | + | + | + | + | + |
| Apo E | ? | 0 | + | −/? | + |
| Complement | + | ? | + | + | + |
| Elastin | + | ? | + | −/? | ? |
| Lipids | −* | ?/− | + | + | + |
| Ca²⁺ | ?** | ? | 0 | ? | + |
| Macrophages | ? | + | + | ? | +/? |

*Sudanophilia has been described with actinic elastosis.
**Calcification of elastic fibers occurs in pseudoxanthoma elasticum.
References for Table 1: Aisen, 1996; Babaev, et al., 1990; Bobryshev, et al., 1995; Castano, et al., 1995; Dahlback, et al., 1988; Dahlback, et al., 1989; Dahlback, et al., 1990; Guyton and Klemp, 1996; Hoque, et al., 1993; Jang, et al., 1993; Jansen, et al., 1993; Li, et al., 1995; Muda, et al., 1988; Namba, et al., 1991; Niculescu, et al., 1987; Niculescu, et al., 1989; Pepys, et al., 1994; Sarks and Sarks, 1989; Stary, et al., 1995; Tarnawski, et al., 1995; Wolter and Falls, 1962.

### III. DIAGNOSTIC ASSAYS

In one aspect, the invention provides a method for diagnosing, or determining a predisposition to developing a drusen associated disease by detecting one or more markers which are associated with drusen development. Examples of phenotypic markers include: RPE dysfunction and/or death, immune mediated events at the RPE-Bruch's membrane-choroid interface, dendritic cell activation, migration and differentiation, extrusion of the dendritic cell process into the sub RPE space (e.g. by detecting the presence or level of a dendritic cell marker such as CD68, CD1a and S100), and the presence of geographic atrophy or disciform scars. Examples of genotypic markers include mutant genes and/or a distinct pattern of differential gene expression (Drusen Development Pathway”), including genes that are upregulated or downregulated in drusen forming ocular tissue associated with drusen biogenesis. For example genes expressed by dysfunctional and/or dying RPE cells include: HLA-DR, CD68, vitronectin, apolipoprotein E, clusterin and S-100. Genes expressed by choroidal and RPE cells in AMD include heat shock protein 70, death protein, proteasome, Cu/Zn superoxide dismutase, cathepsins, and death adaptor protein RAIDD. Markers involved in immune mediated events associated with drusen formation include: autoantibodies (e.g. directed against drusen, RPE and/or retina components), leukocytes, dendritic cells, myofibroblasts, type VI collagen, and a cadre of chemokines and cytokines. Molecules associated with drusen include: immunoglobulins, amyloid A, amyloid P component, HLA-DR, fibrinogen, Factor X, prothrombin, complements 3, 5, 9, and 5b-9, c reactive protein (CRP) apolipoprotein A, apolipoprotein E, antichymotrypsin, β2 microglobulin, thrombospondin, and vitronectin. Markers of drusen associated dendritic cells include: CD1a, CD4, CD 14, CD68, CD83, CD86, and CD45, PECAM, MMP14, ubiquitin, and FGF. Important dendritic cell-associated accessory molecules that participate in T cell recognition include ICAM-1, LFAI, LFA3, and B7, IL-1, IL-6, IL-12, TNF-alpha, GM-CSF and heat shock proteins. Markers associated with dendritic cell expression include: colony stimulating factor, TNFα, and IL-1. Markers associated with dendritic cell proliferation include: GM-CSF, IL-4, IL-3, SCF, FLT-3 and TNFα. Markers associated with dendritic cell differentiation include IL-10, M-CSF, IL-6 and IL-4.

The method can also include a step of detecting drusen-associated markers by one or more ophthalmological procedures. The ophthalmological procedures that can be employed include fundus fluorescein angiography (FFA), fundus ophthalmoscopy or photography (FP), electroretinogram (ERG), electrooculogram (EOG), visual fields, scanning laser ophthalmoscopy (SLO), visual acuity measurements, dark adaptation measurements or other standard methods.

The drusen-associated markers can also be detected on the molecular level, e.g. by detecting the identity, level and/or activity of the gene, mRNA transcript or encoded protein. For example, drusen may be detected by determining the presence of any of the following: amyloid A protein, amyloid P component, antichymotrypsin, apolipoprotein E, β2 microglobulin, complement 3, complement C5, complement CSb-9 terminal complexes, factor X, fibrinogen, immunoglobulins (kappa and lambda), prothrombin and thrombospondin. In another embodiment, the drusen-associated marker is a molecule whose production is altered in a drusen-associated molecular pathological process. For example, one pathological process associated with drusen biogenesis is cell death and/or dysfunction of the retinal pigment epithelium (RPE). A number of molecular markers have been associated with such dysfunctional RPE cells including HLA-DR, CD68i vitronectin, apolipoprotein-E, clusterin and S-100. HLA-DR expression is particularly unique for non-immunocompetent cells (although it is frequently expressed by cells early in an immune reaction). Still other molecular markers associated with dysftnctional choroid and RPE cells of AMD-affected eyes include gene products associated with cell death such as: death protein, heat shock protein 70, proteasome, Cu/Zn superoxide dismutase, cathepsins, and death adaptor protein RAIDD. Furthermore, drusen biogenesis is facilitated by dendritic cells and various immune-mediated events such as the production of autoantibodies in the sera of AMD patients. These autoantibodies are directed against drusen, the RPE and other retinal components.

Accordingly, the invention provides diagnostic assays designed to detect the presence and antigen specificity of such autoantibodies by methods known in the art, including standard imrinunohistochemical and Western blot techniques. Furthermore a number of immune system-associated molecules, including Ig mu, lambda, J, and kappa chains and various cytokines are up-regulated in the RPE/choroid in conjunction with the formation of drusen. Accordingly, these immune-associated molecules provide another target for protein-based (e.g. antibody-based detection methods) and nucleic acid-based (e.g. Northern, and RT-PCR methods) diagnostic assays. Still other drusen-associated molecular markers are those found in conjunction with subpopulation of choroidal cells that possess cellular processes which breach Bruch's membrane and terminate as bulbous, vesicle-filled “cores” within the centers of drusen. Specific marker molecules associated with these dendritic cells include: HLA-DR, CD1a, CD4, CD14, CD68, CD83, CD86 and CD45. Other molecular markers appear to be associated with drusen-associated dendritic cell cores include: PECAM, MMP14, ubiquitin, FGF and HLA. In yet another aspect of the invention, the drusen-associated marker may be a cytokine which facilitates the development of drusen via a receptor-ligand interaction between a dendritic cell precursor and an injured tissue. Such cytokines include: IL-1, IL-6, IL-12, TNF-alpha, and GM-CSF. Other molecules involved in drusen development include heat shock proteins, DNA fragments, angiogenic agents and factors up regulated, such as β integrin, collagen 6α2, collagen 6 α3, elastin, HME, or down regulated (e.g. BIGH3) in diseases characterized by extracellular matrix dysequilibria or associated with immune mediated events.

A variety of means are currently available for detecting aberrant levels or activities of genes and gene products. For example, many methods are available for detecting specific alleles at human polymorphic loci. The preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA TM is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA TM in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g., blood, obtained by known techniques (e.g. venipuncture) or saliva. Alternatively, nucleic acid tests can be performed on dry samples (e.g. hair or skin).

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G.J., 1992, PCR in situ hybridization: protocols and applications, Raven Press, NY).

In addition to methods which focus primarily on the detection of one nucleic acid sequence, gene expression profiles can also be employed in such detection schemes. Gene expression profiles (“fingerprint” profiles) can be generated, for example, by utilizing a differential display procedure, Northern analysis, RT-PCR, and/or gene expression arrays described herein.

A preferred detection method is allele specific hybridization using probes overlapping a region of at least one allele of a drusen associated marker, which has at least about 5, 10, 20, 25, or 30 nucleotides around the mutation or polymorphic region. In a preferred embodiment of the invention, several probes capable of hybridizing specifically to other allelic variants involved in glaucoma are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P.M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of a drusen-associated marker under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, aberrant levels or activities of drusen-associated markers are identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety of sequencing reactions known in the art can be used to directly sequence the allele. Exemplary sequencing reactions include those based on techniques developed by Maxim and Gilbert ((1977) Proc. Natl Acad Sci USA 74:560) or Sanger (Sanger et al (1977) Proc. Nat. Acad. Sci USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetraoxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; and Saleeba et al (1992) Methods Enzymol; 217:286-295. In a-preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, an appropriate probe is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify aberrant levels or activities of drusen-associated markers. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of an allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g.,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect aberrant levels or activities of drusen-associated markers. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microliter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Another embodiment of the invention is directed to kits for detecting a predisposition 25 for developing a drusen-associated ocular disorder. This kit may contain one or more oligonucleotides, including 5′ and 3′ oligonucleotides that hybridize 5′ and 3′ to at least one drusen-associated marker. PCR amplification oligonucleotides should hybridize between 25 and 2500 base pairs apart, preferably between about 100 and about 500 bases apart, in order to produce a PCR product of convenient size for subsequent analysis.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include DNA sampling means. DNA sampling means are well known to one of skill in the art and can include, but not be limited to substrates, such as filter papers, and the like; DNA purification reagents such as Nucleon™ kits, lysis buffers, proteinase solutions and the like; PCR reagents, such as 10× reaction buffers, thermostable polymerase, dNTPs, and the like; and allele detection means such as restriction enzyme, allele specific oligonucleotides, degenerate oligonucleotide primers for nested PCR from dried blood.

### IV. PREDICTIVE MEDICINE

Information obtained using the diagnostic assays described herein (alone or in conjunction with additional genetic or environmental information, which contributes to the drusen associated ocular disorder) may be useful for diagnosing or confirming that a symptomatic subject (e.g. a subject symptomatic for AMD), has a genetic defect (e.g. in an AMD-associated gene or in a gene that regulates the expression of a drusen-associated marker gene), which causes or contributes to the particular disease or disorder. Alternatively, the information can be used prognostically. Based on the prognostic information, a doctor can recommend a regimen (e.g. diet or exercise) or therapeutic protocol, useful for preventing or prolonging onset of the particular disease or condition in the individual.

In addition, knowledge of the particular alteration or alterations, resulting in defective or deficient genes or proteins in an individual (the genetic profile), alone or in conjunction with information on other genetic defects contributing to the same disease (the genetic profile of a drusen associated disease) allows customization of therapy for the particular disease to the individual's genetic profile, the goal of “phannacogenomics”. For example, an individual's genetic profile or the genetic profile of a disease or condition, to which genetic alterations cause or contribute, can enable a doctor to 1) more effectively prescribe a drug that will address the molecular basis of the disease or condition; and 2) better determine the appropriate dosage of a particular drug. For example, the expression level of drusen-associated molecular marker proteins, alone or in conjunction with the expression level of other genes, known to contribute to the same disease, can be measured in many patients at various stages of the disease to generate a transcriptional or expression profile of the disease. Expression patterns of individual patients can then be compared to the expression profile of the disease to determine the appropriate drug and dose to administer to the patient.

The ability to target populations expected to show the highest clinical benefit, based on the genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of a drusen-associated molecular markers can be useful for optimizing effective dose).

### V. SCREENING ASSAYS FOR THERAPEUTICS FOR DRUSEN RELATED OCULAR DISORDERS

A. Cell-Free Assays

Cell-free assays can be used to identify compounds which are capable of interacting with a drusen-associated marker or binding partners thereto, to thereby modify their activity and/or interaction. Such a compound can, e.g., modify the structure of a drusen-associated marker or binding partner thereto and thereby effect its activity.

Accordingly, one exemplary screening assay of the present invention includes the steps of contacting a drusen-associated marker or functional fragment thereof or a binding partner thereto with a test compound or library of test compounds and detecting the presence or absence of complex formation. For detection purposes, the molecule can be labeled with a specific marker and the test compound or library of test compounds labeled with a different marker. Interaction of a test compound with a drusen-associated marker, fragment thereof or a binding partner thereto can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the drusen-associated marker, ftmctional fragment thereof or binding partner thereto is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) a drusen-associated marker, (ii) a binding partner, and (iii) a test compound; and (b) detecting interaction of the drusen-associated marker and binding partner. The drusen-associated marker and binding partner can be produced recombinantly, purified from a source, e.g., plasma, or chemically synthesized, as described herein. A statistically significant change (potentiation or inhibition) in the interaction of the drusen-associated marker and the binding protein in the presence of the test compound, relative to the interaction in the absence of the test compound, indicates a potential agonist (mimetic or potentiator) or antagonist (inhibitor) of drusen-associated bioactivity for the test compound. The compounds of this assay can be contacted simultaneously. Alternatively, a drusen-associated marker can first be contacted with a test compound for an appropriate amount of time, following which the binding partner is added to the reaction mixture. The efficacy of the compound can be assessed by generating dose response curves from data obtained using various concentrations of the test compound. Moreover, a control assay can also be performed to provide a baseline for comparison.

Complex formation between a drusen-associated marker and a binding partner may be detected by a variety of techniques. Modulation of the formation of complexes can be quantitated using, for example, detectably labeled proteins such as radiolabeled, fluorescently labeled, or enzymatically labeled drusen-associated markers or binding partners, by immunoassay, or by chromatographic detection.

Typically, it will be desirable to immobilize either the drusen-associated marker or its binding partner to facilitate separation of complexes from uncomplexed forms of one or both of the proteins, as well as to accommodate automation of the assay. Binding of drusen-associated marker to a binding partner, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase (GST) fusion proteins can be adsorbed onto glutathione sepharose beads (Sigma Chemical, St. Louis, Mo.) or glutathione derivatized microtitre plates, which are then combined with the drusen-associated marker gene product binding partner, e.g. an 35S-labeled drusen-associated marker gene product binding partner, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintillant), or in the supernatant after the complexes are subsequently dissociated. Alternatively, the complexes can be dissociated from the matrix, separated by SDS-PAGE, and the level of drusen-associated marker gene product protein or associated binding partner found in the bead fraction quantitated from the gel using standard electrophoretic techniques such as described in the appended examples.

Other techniques for immobilizing proteins on matrices are also available for use in the subject assay. For instance, either a drusen-associated marker or its cognate binding partner can be immobilized utilizing conjugation of biotin and streptavidin. For instance, biotinylated drusen-associated marker molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with drusen-associated marker can be derivatized to the wells of the plate, and the drusen associated marker trapped in the wells by antibody conjugation. As above, preparations of a drusen-associated marker, a binding partner and a test compound are incubated in the presenting wells of the plate, and the amount of complex trapped in the well can be quantitated. Exemplary methods for detecting such complexes, in addition to those described above for the GST-immobilized complexes, include immunodetection of complexes using antibodies reactive with the drusen-associated marker or binding partner, or which are reactive with the drusen-associated marker and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the drusen-associated marker or binding partner. To illustrate, the drusen-associated marker or binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as anti-drusen-associated marker antibodies, can be used. Alternatively, the protein to be detected in the complex can be “epitope tagged” in the form of a fusion protein which includes, in addition to the drusen-associated marker, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

Cell-free assays can also be used to identify compounds which modulate an activity of an drusen-associated marker. Accordingly, in one embodiment, a drusen-associated marker is contacted with a test compound and the catalytic activity of the drusen-associated marker is monitored. In one embodiment, the ability of a drusen-associated marker to bind a target molecule is determined. The binding affinity of a drusen-associated marker to a target molecule can be determined according to methods known in the art. Determination of the enzymatic activity of a drusen-associated marker can be performed with the aid of the substrate furanacryloyl-L-phenylalanyl-glycyl-glycine (FAPGG) under conditions described in Holmquist et al. (1979) Anal. Biochem. 95:540 and in U.S. Pat. No. 5,259,045.

B. Cell-Based Assays

In addition to cell-free assays, such as described above, drusen-associated markers provided by the present invention facilitate the generation of cell-based assays, e.g., for identifying small molecule agonists or antagonists. In one embodiment, a cell expressing a drusen-associated marker on the outer surface of its cellular membrane is incubated in the presence of a test compound alone or in the presence of a test compound and a drusen-associated marker and the interaction between the test compound and the drusen-associated marker or between the drusen-associated marker and the drusen-associated marker binding partner is detected, e.g., by using a microphysiometer (McConnell et al. (1992) Science 257:1906). An interaction between the drusen-associated marker and either the test compound or the binding partner is detected by the microphysiometer as a change in the acidification of the medium. This assay system thus provides a means of identifying molecular antagonists which, for example, function by interfering with drusen-associated marker—ligand (e.g. receptor) interactions, as well as molecular agonist which, for example, function by activating a drusen-associated marker.

Cell based assays can also be used to identify compounds which modulate expression of a drusen-associated marker gene, modulate translation of a drusen-associated marker mRNA, or which modulate the stability of a drusen-associated marker mRNA or protein. Accordingly, in one embodiment, a cell which is capable of expressing a drusen-associated marker, e.g., a retinal epithelial cell, is incubated with a test compound and the amount of drusen-associated marker produced in the cell medium is measured and compared to that produced from a cell which has not been contacted with the test compound. The specificity of the compound vis a vis a drusen-associated marker can be confirmed by various control analysis, e.g., measuring the expression of one or more control genes. Compounds which can be tested include-small molecules, proteins, and-nucleic acids. In particular, this assay can be used to determine the efficacy of antisense or ribozymes to drusen-associated marker genes.

In another embodiment, the effect of a test compound on transcription of a drusen-associated marker gene is determined by transfection experiments using a reporter gene operatively linked to at least a portion of the promoter of a drusen-associated marker gene. A promoter region of a gene can be isolated, e.g., from a genomic library according to methods known in the art. The reporter gene can be any gene encoding a protein which is readily quantifiable, e.g., the luciferase or CAT gene. Such reporter gene are well known in the art.

This invention further pertains to novel agents identified by the above-described screening assays and uses thereof for treatments as described herein.

C. Animal Models

The invention further provides for animal models, including transgenic animals, which can be used for a variety of purposes, e.g., to identify genetic loci involved in the common etiology of drusen associated diseases, and, further, to create animal models for the treatment of drusen associated diseases.

The transgenic animals can contain a transgene, such as reporter gene, under the control of a drusen-associated marker gene promoter or fragment thereof. These animals are useful, e.g., for identifying drugs that modulate production of the drusen-associated molecular marker, such as by modulating Factor X, HLA-DR, IL-6 or elastin gene expression. A target gene promoter can be isolated, e.g., by screening of a genomic library with an appropriate cDNA fragment and characterized according to methods known in the art. In a preferred embodiment of the present invention, the transgenic animal containing a reporter gene is used to screen a class of bioactive molecules for their ability to modulate expression of a drusen-associated molecular marker such as a DRAM. Yet other non-human animals within the scope of the invention include those in which the expression of the endogenous target gene has been mutated or “knocked out”. A “knock out” animal is one carrying a homozygous or heterozygous deletion of a particular gene or genes. These animals could be useful to determine whether the absence of the target will result in a specific phenotype, in particular whether these mice have or are likely to develop a drusen associated disease. Furthermore these animals are useful in screens for drugs which alleviate or attenuate the disease condition resulting from the mutation of drusen associated markers. These animals are also useful for determining the effect of a specific amino acid difference, or allelic variation, in a target gene. That is, the target knock out animals can be crossed with transgenic animals expressing, e.g., a mutated form-or allelic variant of the target gene containing a drusen associated marker, thereby resulting in an animal which expresses only the mutated protein and not the wild-type target gene product.

Methods for obtaining transgenic and knockout non-human animals are well known in the art. Knock out mice are generated by homologous integration of a “knock out” construct into a mouse embryonic stem cell chromosome which encodes the gene to be knocked out. In one embodiment, gene targeting, which is a method of using homologous recombination to modify an animal's genome, can be used to introduce changes into cultured embryonic stem cells. By targeting a specific gene of interest in ES cells, these changes can be introduced into the germlines of animals to generate chimeras. The gene targeting procedure is accomplished by introducing into tissue culture cells a DNA targeting construct that includes a segment homologous to a target locus, and which also includes an intended sequence modification to the genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting a target gene function through the use of a targeting transgene construct designed to undergo homologous recombination with one or more target genomic sequences. The targeting construct can be arranged so that, upon recombination with an element of a target gene, a positive selection marker is inserted into (or replaces) coding sequences of the gene. The inserted sequence functionally disrupts the target gene, while also providing a positive selection trait. Exemplary targeting constructs are described in more detail below.

Generally, the embryonic stem cells (ES cells ) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. Mol Biol. 87:27-45). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934) Still another preferred ES cell line is the WW6 cell line (loffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E.J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. [1986].

A knock out construct refers to a uniquely configured fragment of nucleic acid which is introduced into a stem cell line and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art. A typical knock out construct contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid which encodes a positive selectable marker, such as the neomycin resistance gene (neoR). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for the gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding-the positive-selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgenic animal which expresses a polypeptide of the targeted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. A marker gene is one that encodes an enzyme whose activity can be detected (e.g., b-galactosidase), the enzyme substrate can be added to the cells under suitable conditions, and the enzymatic activity can be analyzed. One skilled in the art will be familiar with other useful markers and the means for detecting their presence in a given cell. All such markers are contemplated as being included within the scope of the teaching of this invention.

As mentioned above, the homologous recombination of the above described “knock out” and “knock in” constructs is very rare and frequently such a construct inserts nonhomologously into a random region of the genome where it has no effect on the gene which has been targeted for deletion, and where it can potentially recombine so as to disrupt another gene which was otherwise not intended to be altered. Such nonhomologous recombination events can be selected against by modifying the above-mentioned knock out and knock in constructs so that they are flanked by negative selectable markers at either end (particularly through the use of two allelic variants of the thymidine kinase gene, the polypeptide product of which can be selected against in expressing cell lines in an appropriate tissue culture medium well known in the art—i.e. one containing a drug such as 5-bromodeoxyuridine). Thus a preferred embodiment of such a knock out or knock in construct of the invention consist of a nucleic acid encoding a negative selectable marker linked to a nucleic acid encoding a 5′ end of a genomic locus linked to a nucleic acid of a positive selectable marker which in turn is linked to a nucleic acid encoding a 3′ end of the same genomic locus which in turn is linked to a second nucleic acid encoding a negative selectable marker Nonhomologous recombination between the resulting knock out construct and the genome will usually result in the stable integration of one or both of these negative selectable marker genes and hence cells which have undergone nonhomologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques which are well known to those familiar in the art. Alternatively, PCR can be used.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. For example, if the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knock out construct as explained above. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., Bradley et al. (supra).

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the Target gene knocked out in various tissues of the offspring by probing the Western blot with an antibody against the particular target protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a target-gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

A target transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a target protein can be essential for many forms of lineage analysis and can additionally provide a means to assess the effects of, for example, lack of target expression which might grossly alter development in small patches of tissue within an otherwise normal embryo. Toward this end, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject target proteins. For example excision of a target sequence which interferes with the expression of a recombinant target gene, such as one which encodes an antagonistic homolog or an antisense transcript, can be designed to activate expression of that gene. This interference with expression of the protein can result from a variety of mechanisms, such as spatial separation of the target gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxp sequences are oriented as direct repeats and catalyzes inversion of the target sequence when loxp sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant target protein can be regulated via control of recombinase expression.

Use of the cre/loxP recombinase system to regulate expression of a recombinant target protein requires the construction of a transgenic animal containing transgenes encoding both the Cre recombinase and the subject protein. Animals containing both the Cre recombinase and a recombinant target gene can be provided through the construction of “double” transgenic animals. A convenient method for providing such animals is to mate two transgenic animals each containing a transgene, e.g., a target gene and recombinase gene.

One advantage derived from initially constructing transgenic animals containing a target transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic target transgene is silent will allow the study of progeny from that founder in which disruption of target mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the target transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g. a recombinase or a prokayotic protein, is delivered to the tissue and caused to be expressed, such as in a cell-type specific manner. By this method, a target transgene could remain silent into adulthood until “turned on” by the introduction of the trans-activator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal-used to practice this-invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a Target protein (either agonistic or antagonistic), and antisense transcript, or a Target mutant. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

VI. Therapeutics

A. Therapeutic Compositions and Methods of Treatment

The present invention also provides compositions and methods for treating or preventing the development of drusen associated ocular disorders.

Appropriate therapeutics can include any molecule or compound that slows or prevents any of the processes involved in drusen biogenesis, including dendritic cell activation and recruitment, T cell activation, antigen presentation, dendritic cell-T cell interaction, immune mediated events, autoimmune events, neovascularization, and extracellular matrix disequilibrium, etc. Useful therapeutics include agents that inhibit inflammation. For example, an appropriate therapeutic may be an anti-inflammatory agent, such as an antagonist of TNF-α, IL-1, GM-CSF, IL-4 or IL-13. The therapeutic may also be IL-10, M-CSF, IL-6 and IL-4 or an agonist thereof Any therapeutic that helps to decrease drusen formation or DS/CNV may be used. In a preferred embodiment, the agent is selected from the group consisting of cytokines, chemokines and agonists and antagonists thereof.

In another embodiment, the macular degeneration therapeutic is an inhibitor of the expression of one or more DRAMs, such as, for example, amyloid A protein, amyloid P component, al-antichymotrypsin, apolipoprotein E, P2 microglobulin, complement 3, complement C5, complement C5b-9 terminal complexes, factor X, fibrinogen, immunoglobulins (kappa and lambda), prothrombin, thrombospondin or vitronectin. In an another embodiment, the invention provides method for treating a drusen associated disease by modulating the production of DRAMs, e.g., inhibiting or antagonizing their gene expression or activity. The accumulation of amyloid P and al-antichymotrypsin (an inhibitor of serine proteases) in drusen may act to counterbalance attempts by RPE or choroidal cells to clear drusen proteolytically. For example, amyloid P is also found in non-amyloid deposits associated with atherosclerosis (Niculescu, et al., 1987), keratin intermediate filament aggregates (Hintner, et al., 1988), and dense deposits associated with glomerulonephropathy (Yang, et al., 1992). It associates with elastic fibers and may fuiction as an protease inhibitor in vivo (Li and McAdam, 1984; Vachino, et al., 1988). It is also a normal component of Bruch's membrane, where it might protect the elastic lamina against enzymatic degradation (Kivela, et al., 1994). The downregulation of the biosynthesis of these proteins is therefore important for inhibiting drusen formation or facilitating drusen clearance or resolution. Inhibiting of drusen formation or facilitating drusen clearance or resolution may be accomplished by a number of regimes, such as (1) inhibition of RNA synthesis for one or more DRAMs, (2) enhancement of RNA turnover or degradation of one or more DRAMs, (3) inhibition of translation of RNA for one or more DRAMs into protein, (4) inhibition of protein processing or transport of one or more DRAMs; (5) inhibition of drusen formation by blocking particular protein binding sites on one or more factors which participate in inter- and intra-molecular binding necessary for the association of DRAMs which results in a drusen deposit; (6) digestion or perturbation of protein deposits (e.g., using enzymes); (7) targeting and destroying DRAMs in situ (e.g., using enzyme-antibody techniques). DRAMs may be targeted by using photoreactive laser therapy, for example, or other means for targeting and destroying a protein in situ which are well known in the art. Such means may include antibodies conjugated to a reactive group such as a protease or chemical substance which, when activated, cleaves or denatures the individual components or interferes with the interaction of two or more components.

In another embodiment, therapeutics for drusen-associated diseases include agents which alter the gene expression of factors that regulate the expression of one or more DRAMs and all other drusen biogenesis associated proteins. Such agents may be “antagonists” which inhibit, either directly or indirectly, DRAM biosynthesis. The agent may specifically inhibit the transcription or translation of a DRAM, for example. Alternatively, it may be preferable to upregulate either directly or indirectly a gene or genes which will increase the synthesis of a naturally occurring therapeutic agent. For example, the increased gene expression of a proteolytic enzyme that degrades one or more DRAMS or a cytokine or drug that modulates immune responses may be desired.

The invention is therefore also useful for monitoring the efficacy of a drusen therapeutic or preventative treatment, the absence of drusen core formation, the disappearance of drusen or of a drusen core providing evidence of efficacy of the therapeutic or treatment.

In one aspect, the therapeutics of the invention relate to antisense therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more DRAMs so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a DRAM protein. Alternatively, the antisense construct can be an oligonucleotide probe which is generated ex vivo and which, when introduced into the cell causes inhibition of expression by hybridizing with the mRNA and/or genomic sequences of a DRAM gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996, 5,264,564 and 5,256,775). Approaches to constructing oligomers useful in antisense therapy are well known in the art. With respect to antisense DNA, oligodeoxyribonucleotides derived from the translation initiation site, e.g., between the −10 and +10 regions of the drusen-associated component nucleotide sequence of interest, are preferred.

Antisense approaches involve the design of oligonucleotides (either DNA or RNA) that are complementary to a DRAM mRNA, or their agonists or antagonists. The antisense oligonucleotides bind to the subject mRNA transcripts and prevent translation or promote degradation of the transcript. Absolute complementarity, although preferred, is not required. In the case of double-stranded antisense nucleic acids, a single strand of the duplex DNA may thus be tested, or triplex formation may be assayed. The ability to hybridize depends on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex. Other features, strategies and methods of preparing and using antisense or ribozymes are found in U.S. Ser. No. 09/183,972, the teachings of which are incorporated herein.

B. Gene Therapy

Therapeutics of the present invention also include gene therapy-based regimes. Gene therapy refers to the introduction of an otherwise exogenous polynucleotide which produces a medically useful phenotypic effect upon the (typically) mammalian cell(s) into which it is transferred. According to the present invention, a gene delivery system for a DRAM polynucleotide therapeutic can be introduced into a patient by any of a number of methods. For example, the gene delivery vehicle can be introduced by catheter (See U.S. Pat. No. 5,328,470) or by stereotactic injection (Chen et al. (1994), Proc. Natl. Acad. Sci. USA 91:3054-3057. A DRAM gene or cDNA can be delivered in a gene therapy construct by electroporation using techniques described. Dev. et al. (1994), Cancer Treat. Rev. 20:105-115.

In some methods, gene therapy involves introducing into a cell a vector that expresses a DRAM gene product (e.g., a DRAM polypeptide), expresses a nucleic acid having a DRAM gene or mRNA sequence (e.g., an antisense RNA), expresses a polypeptide or polynucleotide that otherwise affects expression of DRAM gene products (e.g., a ribozyme directed to a DRAM mRNA), or replaces or disrupts an endogenous DRAM sequence (e.g., gene replacement and gene knockout, respectively). Numerous other embodiments will be evident to one of skill upon review of the disclosure herein.

Vectors useful in DRAM gene therapy can be viral or nonviral. Gene therapy vectors can comprise promoters and other regulatory or processing sequences, such as are described in this disclosure. Usually the vector will comprise a promoter and, optionally, an enhancer (separate from any contained within the promoter sequences) that serve to drive transcription of an oligoribonucleotide, as well as other regulatory elements that provide for episomal maintenance or chromosomal integration and for high-level transcription, if desired. A plasmid useful for gene therapy can comprise other functional elements, such as selectable markers, identification regions, and other sequences. The additional sequences can have roles in conferring stability both outside and within a cell, targeting delivery of DRAM nucleotide sequences (sense or antisense) to a specified organ, tissue, or cell population, mediating entry into a cell, mediating entry into the nucleus of a cell and/or mediating integration within nuclear DNA. For example, aptamer-like DNA structures, or other protein binding moieties sites can be used to mediate binding of a vector to cell surface receptors or to serum proteins that bind to a receptor thereby increasing the efficiency of DNA transfer into the cell. Other DNA sites and structures can directly or indirectly bind to receptors in the nuclear membrane or to other proteins that go into the nucleus, thereby facilitating nuclear uptake of a vector. Other DNA sequences can directly or indirectly affect the efficiency of integration.

Suitable gene therapy vectors can have an origin of replication. For example, it is useful to include an origin of replication in a vector for propagation of the vector prior to administration to a patient. However, the origin of replication can often be removed before administration if the vector is designed to integrate into host chromosomal DNA or bind to host mRNA or DNA.

The present invention also provides methods and reagents for gene replacement therapy (i.e., replacement by homologous recombination of an endogenous DRAM gene with a recombinant gene). Vectors specifically designed for integration by homologous recombination may be used. Important factors for optimizing homologous recombination include the degree of sequence identity and length of homology to chromosomal sequences. The specific sequence mediating homologous recombination is also important, because integration occurs much more easily in transcriptionally active DNA. Methods and materials for constructing homologous targeting constructs are described by e.g., Mansour et al., 1988, Nature 336: 348; Bradley et al., 1992, Bio/Technology 10: 534. See also, U.S. Pat. Nos. 5,627,059; 5,487,992; 5,631,153; and 5,464,764. In one embodiment, gene replacement therapy involves altering or replacing all or a portion of the regulatory sequences controlling expression of a DRAM gene that is to be regulated. For example, the promoter sequences may be disrupted (to decrease the DRAM expression or to abolish a transcriptional control site) or an exogenous promoter (e.g., to increase the DRAM expression) substituted.

The invention also provides methods and reagents for DRAM gene knockout (i.e., deletion or disruption by homologous recombination of an endogenous DRAM gene using a recombinantly produced vector). In gene knockout, the targeted sequences can be regulatory sequences (e.g., a DRAM promoter), or RNA or protein coding sequences. The use of homologous recombination to alter expression of endogenous genes is described in detail in U.S. Pat. No. 5,272,071 (and the U.S. Patents cited supra), WO 91/09955, WO 93/09222, WO 96/29411, WO 95/31560, and WO 91/12650. See also, Moynahan et al., 1996, Hum. Mol. Genet. 5:875.

Gene therapy vectors can be introduced into cells or tissues in vivo, in vitro or ex vivo. For ex vivo therapy, vectors may be introduced into cells, e.g., stem cells, taken from the patient and clonally propagated for autologous transplant back into the same patient (see, e.g., U.S. Pat. Nos. 5,399,493 and 5,437,994, the disclosures of which are herein incorporated by reference).

C. Formulation and Use

Pharmaceutical compositions for use in accordance with the present invention may be formulated in a conventional manner using one or more physiologically acceptable carriers or excipients. Thus, the compounds and their physiologically acceptable salts and solvates may be formulated for administration by, for example, eye drops, injection, inhalation or insufflation (either through the mouth or the nose) or oral, buccal, parenteral or rectal administration.

For such therapy, the compounds of the invention can be formulated for a variety of modes of administration, including systemic and topical or localized administration. Techniques and formulations generally may be found in Remmington's Pharmaceutical Sciences, Meade Publishing Co., Easton, Pa. A preferred method of administration is an eye drop. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

Other preferred methods of administration include choroidal injection, transscleral injection or placing a scleral patch, and selective arterial catheterization. Other preferred deliveries are intraocular, including transretinal, subconjunctival bulbar, scleral pocket and scleral cutdown injections. The agent can be alternatively administered intravascularly, such as intravenously (IV) or intraarterially.

Techniques for choroidal injection and scleral patching are similar. The clinician uses a local approach to the eye after initiation of appropriate anesthesia, including painkillers and ophthalmoplegics. A needle containing the therapeutic compound is directed into the patient's choroid or sclera and inserted under sterile conditions. When the needle is properly positioned the compound is injected into either or both of the choroid or sclera. When using either of these methods, the clinician may choose a sustained release or longer acting formulation. Thus, the procedure may need repetition only every several months or several years, depending on the patient's tolerance of the treatment and response.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The therapeutic may be administered alone or in combination with other molecules known to have a beneficial effect on retinal attachment or damaged retinal tissue, including molecules capable of tissue repair and regeneration and/or inhibiting inflammation. Examples of useful cofactors include basic fibroblast growth factor (bFGF), LaVail et al. (1998), Invest. Ophthalmol. Vis. Sci. 39:592-602, ciliary neurotrophic factor (CNTF), LaVail et al. (1998), Invest. Ophthalmol. Vis. Sci. 39:592-602, axokine (a mutein of CNTF), LaVail et al. (1998), Invest. Ophthalmol. Vis. Sci. 39:592-602, leukemia inhibitory factor (LIF), LaVail et al. (1998), Invest. Ophthalmol. Vis. Sci. 39:592-602. neutrotrophin 3 (NT-3), LaVail et al. (1998), Invest. Ophthalmol. Vis. Sci. 39:592-602, neurotrophin-4 (NT-4), LaVail et al. (1998), Invest. Ophthalmol. Vis. Sci. 39:592-602, nerve growth factor (NGF), LaVail et al. (1998), Invest. Ophthalmol. Vis. Sci. 39:592-602, insulin-like growth factor II, LaVail et al. (1998), Invest. Ophthalmol. Vis. Sci. 39:592-602, prostaglandin E2, La Vail et al. (1998), Invest. Ophthalmol. Vis. Sci. 39:581-591, 3OkD survival factor, taurine, and vitamin A. Other useful cofactors include symptom-alleviating cofactors, including antiseptics, antibiotics, antiviral and antifungal agents and analgesics and anesthetics.

A therapeutic also may be associated with means for targeting the therapeutics to a desired tissue. Alternatively, an antibody or other binding protein that interacts specifically with a surface molecule on the desired target tissue cells also may be used. Such targeting molecules further may be covalently associated to a therapeutic, e.g., by chemical crosslinking, or by using standard genetic engineering means to create, for example, an acid labile bond such as an Asp-Pro linkage. Useful targeting molecules may be designed, for example, using the simple chain binding site technology disclosed, for example, in U.S. Pat. No. 5,091,513.

Preparations for oral administration may be suitably formulated to give controlled release of the active compound. For buccal administration, the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt. Other suitable delivery systems include microspheres which offer the possibility of local noninvasive delivery of drugs over an extended period of time. This technology utilizes microspheres of precapillary size which can be injected via a coronary catheter into any selected part of the body, e.g., the eye, or other organs without causing inflammation or ischemia. The administered therapeutic is slowly released from these microspheres and taken up by surrounding tissue cells (e.g., endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

As described above, a gene delivery system for a gene therapeutic can be introduced into a patient by any of a number of well known methods in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter, See U.S. Pat. 5,328,470, or by stereotactic injection, Chen et al. (1994), Proc. Natl. Acad. Sci., USA 91: 3054-3057. A sequence homologous thereto can be delivered in a gene therapy construct by electroporation using techniques described, Dev et al. (1994), Cancer Treat. Rev. 20:105-115.

The pharmaceutical preparation of the gene therapy construct or compound of the invention can consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

D. Effective Dose

Toxicity and therapeutic efficacy of such compounds can be determined by standard pharmaceutical procedures in cell cultures or experimental animals, e.g., for determining the Ld50 (the dose lethal to 50% of the population) and the Ed50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The practice of the present invention can employ, unless otherwise indicated, conventional techniques of cell biology, cell culture, molecular biology, transgenic biology, microbiology, recombinant DNA, and immunology, which are within the skill of the art. Such techniques are explained fully in the literature. Molecular Cloning A Laboratory Manual (1989), 2nd Ed., ed. by Sambrook, Fritsch and Maniatis, eds., Cold Spring Harbor Laboratory Press, Chapters 16 and 17; Hogan et al. (Manipulating the Mouse Embryo: A Laboratory Manual (1986), Cold Spring Harbor Laboratory Press, Cold Spring Harbor, N.Y.; See U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The present invention is further illustrated by the following examples which should not be construed as limiting in any way. The contents of all cited references (including literature references, issued patents, published patent applications as cited throughout this application) are hereby expressly incorporated by reference.

### VII. REFERENCES CITED

Abremski et al. (1984), J. Biol. Chem,. 259, 1509-1514.

Abravaya et al. (1995), Nuc. Acid. Res.,. 23, 675-682.

Akiyama, H., et al. (1991), J. Neuroimmunol., 32, 19-28.

Allikmets, R., et al. (1997), Science, 277, 1805-7.

Alt, A. (1877), Arch. Ophthalmol. and Otolaryngol., 6, 304-323.

Babaev, V., et al. (1990), Atherosclerosis, 85, 239-247.

Badimon, J., et al. (1993), Circulation, 87 (3 Suppl), II3-116.

Barany (1991), Proc. Natl. Acad. Sci. USA, 88, 189.

Been and Cech (1986), Cell, 47, 207-216.

Bernoist and Chambon (1981), Nature, 290, 304-310.

Bilato amd Crow (1996) Aging 8(4):221-34

Bird, A. (1992), Pathophysiology of AMD. In G. Hampton and N. PT (Eds.), Age-related macular degeneration: principles and practice (pp. chap. 3.). New York: Raven Press.

Bird, A. (1992a), Br. J. Ophthalmol., 763, 166-168.

Bird, A. (1992b), Pathophysiology of AMD. In G. Hampton and N. PT (Eds.), Age-related macular degeneration: principles and practice (pp. chap. 3.). New York: Raven Press.

Bird, A. (1996), Br. J. Ophthalmol., 80, 2-3.

Bird, A. (1997), The Wolfe Lecture: Genotype/phenotype correlations in hereditary macular dystrophies No. The University of Iowa.

Bird, A., et al. (1995), Survey of Ophthal., 39(5), 367-74.

Bittorf, S. V., et al. (1993), J. Biol. Chem., 268, 24838-24846.

Blumenkranz, M., et al. (1986), Ophthalmol., 93, 552-558.

Bobryshev, Y., et al. (1995), Atherosclerosis, 118, 9-21.

Bobryshev, Y.V., and Lord, R.S. (1995), Cardiovas. Res. 29, 689-696.

Bocan, T., et al. (1988), Arteriosclerosis, 8, 499-508.

Bradley et al. (1984), Nature, 309,255-258.

Bradley et al. (1986), Current Topics Devel. Biol., 20, 357-371.

Bressler, N., et al. (1994), Retina, 14, 130-142.

Bressler, S., et al. (1990), Arch. of Ophthalmol., 108, 1442-1447.

Brinster et al. (1982), Nature, 296, 39-42.

Brinster et al. (1985), Proc. Natl. Acad. Sci. USA, 82, 4438-4442.

Brown, B., and Lovie-Kitchin, J. (1983), Amer. J. Optom. Physiol. Opt., 60, 645-650.

Brown, B., et al. (1986), Ophthalmic Physiol. Opt., 6, 81-84.

Bunchereau, J., and Steinman, R.M. (1998), Nature 393, 245-252.

Burns, R., and Feeney-Burns, L. (1980), Transactions of the American Ophthalmologic Society, 78, 206-225.

Butler (1981), Meth. Enzymol., 73, 482-523.

Capon, M., et al. (1989), Ophthalmol., 96, 1769-1777.

Caux et al. (1999) “Developmental Pathways in Human Myeloid Dendritic Cells', in Dendritic Cells, Lotze, M.T. and Thomson, A.W. (Eds.) Academic Press, pp. 63-2.

Chang, B., et al. (1996), Invest. Ophthalmol. Vis. Sci. (Suppl), 37, S305.

Chen, J., et al. (1992), Invest Ophthalmol. Vis. Sci., 33, 334-340.

Chen et al. (1994), Proc. Natl. Acad. Sci., USA, 91, 3054-3057.

Chirgwin, J., et al. (1979), Biochem., 18, 5294-5299.

Christen, W., et al. (1997), Invest. Ophthalmol. Vis. Sci. (Suppl.), 38, S472.

Cohen et al. (1996), Adv. Chromatogr.,. 36, 127-162.

Cole et al. (1985), Monoclonal Antibodies and Cancer Therapy, Alan R. Liss, Inc. pp. 77-96.

Collier, L., et al. (1986), Invest. Ophthalmol. Vis. Sci., 27, 702-707.

Coria, F., et al. (1988), Lab. Invest., 58, 454-458.

Cotton et al (1988), Proc. Natl. Acad. Sci. USA, 85, 4397.

Cotton (1993), Mutat. Res., 285, 125-144.

Cronin et al. (1996), Hurnan Mutation, 7, 244.

Cruickshanks, K., et al. (1997), Arch. Ophthalmol., 115, 242-250.

Cruickshanks, K., et al. (1993), Arch. Ophthalmol., 111, 514-518.

Curcio, C., et al. (1996), Invest. Ophthahnol. Vis. Sci., 37, 1236-49.

Curcio, C., et al. (1993), Invest. Ophthalmol Vis. Sci., 34, 3278-3296.

Cutler, J., and AH, P. (1981), Infection and Immunity, 31(3), 1110-1116.

Dahlback, K., et al. (1990), J. Invest. Dennatol., 94, 284-291.

Dahlback, K., et al. (1988), Acta Derm. Venereol., 68, 107-115.

Dahlback, K., et al. (1989), J. Invest. Dermatol., 92, 727-733.

Dahlback, K., et al. (1993), J. Invest. Dermatol., 100, 166-170.

Das and Frank (1990) Arch Ophthalmol 108 (3):421-9.

Dastgheib, K. and Green, R. (1994) Arch. Opthalmol; 112, 813-818.

De La Paz, M., et al. (1998), Invest. Ophthalmol. Vis. Sci. (Suppl), 39, S915.

Deutman, A., and Jansen, L. (1970), Br. J. Ophthahnol., 54, 373-382.

Dev et al. (1994), Cancer Treat. Rev., 20, 105-115.

Doetschman et al. (1985), J. Embryol. Exp. Mol. Biol., 87, 27-45.

Donders, F. (1854), Graefe's Arch. Clin. Exp. OphthaLnol., 1, 106-118.

Dorey, C., et al. (1989), Invest. Ophthalmol. Vis. Sci., 30, 1691-1699.

Duke-Elder, S., and Dobree, J. (1967), Diseases of the Retina. St. Louis: CV Mosby.

Duvall, J., and Tso, M. (1985), Arch. OphthaLnol., 103, 694-703.

Duvall-Young, J., et al. (1989), Br. J. Ophthalmol., 73, 297-302.

Eagle, R. (1984), Ophthalmol., 91, 613-25.

Eisner, A., et al. (1987), Invest. Ophthalmol. Vis. Sci., 28, 1832-1837.

El Baba, F., et al. (1986a), Arner. J. Ophthalmol., 101, 576-83.

El Baba, F., et al. (1986b), Ophthalmol., 93, 1581-1592.

Eglom et al. (1993), Nature, 365, 566.

Ellison et al. (1991), J. Biol. Chem., 266, 21150-21157.

Elman, M., and Fine, S. (1989), In S. Ryan (Ed.), The Retina (pp. 175-200). St Louis: CV Mosby.

Elner, S. G., et al. (1992), Exp. Eye Res., 55(1), 21-8.

Engel, H., et al. (1988), Ophthalmologica, 196, 143-150.

Ermilov, V., and Vodovozov, A. (1995), Vestnik Oftalmologii, 111(4), 24-7.

Evans et al. (1981), Nature, 292, 154-156.

Fariss, R. N., et al. (1997), Amer. J. Path., 150(1), 323-8.

Farkas, T., et al. (1971a), Amer. J. Ophthatmol., 71, 1196-1205.

Farkas, T., et al. (1971b), Amer. J. Ophthalmol., 71, 1206-1215.

Farkas, T and Krill (1971) Trans Am Acad Ophthalmol Otolaryngol 75:333-343.

Feeney-Burns, L., and Ellersieck, M. (1985), Amer. J. Ophthalmol., 100, 686-697.

Feeney-Burns, L., et al. (1988), Invest. Ophthalmol. Vis. Sci., 29, 708-719.

Feeney-Burns, et al. (1987), Invest. Ophthalmol. Vis. Sci., 28, 1138-1147.

Feher and Valu (1967) Albrecht Von Graefes Arch Klin Exp Ophthalmol 173: 162-7.

Figueroa, M., et al. (1994), Retina, 14, 391-396.

Fine, B. (1981), Amer. J. Ophthatmol., 91, 469-473.

Fite, K., Bengston, C., and Cousins, F. (1994), Exper. Eye Res., 59, 417-424.

Fox, P., et al. (1995), Life Sci., 56, 1749-58.

Frenneson, I., and Nilsson, S. (1996), Eur. J. Ophthalmol., 6, 307-314.

Frennesson, C., et al. (1995), Documenta Ophthalmologica, 90, 37786.

Frennesson, I., and Nilsson, S. (1995), Br. J. Ophthalmol., 79, 905-9.

Freshney, R. I. (1987), Culture Of Animal Cells, Alan R. Liss, Inc.

Friedman, E., et al. (1963), Arch. Ophthalmol., 69, 220-230.

Fuchs, U., et al. (1991), Invest. Ophthalmol. Vis. Sci., 32(13), 3178-86.

Gait, M. J. (ed.) (1984) Oligonucleotide Synthesis.

Gasparini et al. (1992), Mol. Cell Probes, 6, 1.

Gass, J. (1973), Arch. Ophthalmol., 90, 206-217.

Gass, J. (1987), Stereoscopic Atlas of Macular Diseases: Diagnosis and Treatment (3rd ed.). St Louis: CV Mosby.

Galy, A. (1999), “Hemaopoietic Cell Fate and the Development of Dendritic Cel” in Dendritic Cells, Lotze, M. T. and Thomson, A. W. (Eds.) Academic Press, pp. 3-14

Gautier et al. (1987), Nucl. Acids Res., 15, 6625-6641.

Genge, B., et al. (1991), J. Biol. Chem., 266, 10678-10685.

Ghadially, F. (1988), Ultrastructural Pathology of the Cell and Matrix (3rd ed.). New York: Butterworth.

Gibbs et al. (1989), Nucl. Acids Res., 17, 2437-2448.

Glover, (ed.) (1985) DNA Cloning, Volumes I and II.

Gossler et al. (1986), Proc. Natl. Acad. Sci. USA, 83, 9065-9069.

Green, W., and Enger, C. (1993), Ophthalnol., 100, 1519-1535.

Green, W., and Key, S. (1977), Transactions Amer. Ophthalmol. Soc., 75, 180-254.

Green, W., et al. (1985), Ophthalmol., 92, 615-27.

Griffin et al. (1993), Appl. Biochem. Biotechnol., 38, 147-159.

Guatelli et al. (1990), Proc. Natl. Acad. Sci. USA, 87, 1874-1878.

Gueme, D., et al. (1991), Ophthalmol., 98, 602-7.

Guo, et al., (1997) IOVS (Suppl) 38:S354.

Guymer and Bird (1998) In “The Retinal Pigment Epithelium” MF. Marmor and T. J. Wolfensberger, eds, pp693-705. Oxford University Press, NY.

Guyton, J., and Klemp, K. (1996), Arterioscler., Thromb. and Vas. Biol., 16, 4-11.

Habig et al (1974), J. Biol. Chem., 249, 7130.

Hale, I., and Matsumoto, B. (1993), Methods in Cell Biol., 38, 289-324.

Hames, B. D. and S. J. Higgins, (eds.) (1984) Nucleic Acid Hybridization.

Hames, B. D. and S. J. Higgins, (eds.) (1984), Transcription and Translation.

Hammond, B. J., et al. (1997), Invest. Ophthalmol. Vis. Sci., 38, 1795-801.

Harlow and Lane (eds.) (1988) Antibodies: A Laboratory Manual, Cold Spring Harbor Press.

Haseloffand Gerlach (1988), Nature, 334, 585-591.

Hashimoto, K., and DiBella, R. (1967), J. Invest. Dermatol., 48, 405-422.

Hayashi (1992), Genet. Anal. Tech. Appl., 9, 73-79

Hayman, E., et al. (1983), Proc. Nat. Acad. Sci. USA, 80, 4003-7.

Heiba, I., Elston, et al (1994), Genet. Epidemiol., 11, 51-67.

Helene (1991), Anticancer Drug Des., 6, 569-584.

Helene et al. (1992), Ann. N.Y. Acad. Sci., 660, 27-36.

Heon, E., et al. (1996), Arch. Ophthalmol., 114, 193-8.

Herkner, K., et al. (1992), Clin. Chem., 38, 548-50.

Hewitt, A., and Newsome, D. (1985), Cur. Eye Res., 4, 169-173.

Hintner, H., et al. (1988), J. Invest. Derm., 91, 22-28.

Hirata, A., and Feeney-Burns, L. (1992), Invest. Ophthalmol. Vis. Sci., 33, 2079-2090.

Hogan, M. (1972), Transactions Amer. Acad. Ophthalmol. and Otolaryngol., 76, 64-80.

Hogan et al. (1986), Manipulating the Mouse Embryo: A Laboratory Manual, Cold Spring Harbor Laboratory Press, Cold Spring Harbor, N.Y.

Hogasen, K., et al. (1992), J. Biol. Chem., 267, 23076-23082.

Holmquist et al. (1979), Anal. Biochem., 95, 540.

Holz, F., et al. (1995a), Arch. Ophthalmol., 113(2), 178-84.

Holz, F., et al. (1995), German J. Ophthalmol., 4(6), 336-41.

Holz, F., et al. (1994), Arch. Ophthalmol., 112, 402-406.

Hope, G., et al. (1992), Br. J. Ophthalmol., 76, 11-16.

Hsu et al. (1994), Carcinogenesis, 15, 1657-1662.

Hussain, A., et al. (1997), Invest. Ophthalmol. Vis. Sci., 38, S353.

Hyman, L. (1992) Epidemiology of AMD; In G. Hampton and P. Nelsen (Eds.), Age-related Macular Degeneration: Principles and Practice (pp. 1-36). New York: Raven Press.

Hyman, L., et al. (1983), Amer. J. Epidemiol., 118,213-227.

Inoue et al. (1987), Nucl. Acids Res., 15, 6131-6148.

Inoue et al. (1987), FEBS Lett,. 215, 327-330.

Ioffe et al. (1995), Proc. Natl. Acad. Sci., USA, 92, 7357-7361.

Ishibashi, T., et al. (1986a), Amer. J. Ophthalmol., 101, 342-353.

Ishibashi, T., et al. (1986b), Invest. Ophthalmol. Vis. Sci., 27, 184-193.

Ishikawa, et al. (eds.) (1981), Enzyme Immunoassay, Kgaku Shoin, Tokyo.

Jacobson, S., et al. (1995), Nature Genet., 11, 27-32.

Jaenich (1976), Proc. Natl. Acad. Sci. USA, 73, 1260-1264.

Jaenisch (1988), Science, 240, 1468-1474.

Jang, I., et al. (1993), Eur. Heart J., 14(Supplement K), 2-6.

Jahner et al. (1982), Nature, 298, 623-628.

Jahner et al. (1985), Proc. Natl. Acad. Sci. USA, 82, 6927-6931.

Jansen, J., et al. (1993), Amer. J. Path., 143, 1356-65.

Jaworowski, A., et al. (1995), Biochim. Biophys. Acta, 1245(1), 121-9.

Joh, K., et al. (1993), Acta Pathologica Japonica, 43, 552-565.

Johnson, L., and Blanks, J. (1984), Cur. Eye Res., 3, 969-974.

Johnson, L., and Hageman, G. (1987), Exper. Eye Res., 44, 553-565.

Kahn, H., et al. (1977), Amer. J. Epidem., 106, 17-32.

Keen et al. (1991), Trends Genet., 7, 5.

Kida, E., et al. (1994), Neur. Lett., 167, 73-76.

Killingsworth, M., et al. (1990), Eye, 4, 613-621.

Kincaid, M. (1992), Pathology of AMD. In G. Hampton and P. Nelsen (Eds.), Age-related macular degeneration: principles and practice (pp. chap. 2). New York: Raven Press.

Kishimoto, T., et al. (1997), Leukocyte Typing VI. New York: Garland Publishing.

Kivela, T. (1990), Cur. Eye Res., 9, 1195-1209.

Kivela, T., et al. (1994), Invest. Ophthahnol. Vis. Sci., 35, 3759-3769.

Klein, M. (1991), JAMA, 266, 2279.

Klein, M., et al. (1994), Arch. Ophthalmol., 112(7), 932-7.

Klein, R., et al. (1991), Ophthalmol., 98, 1128-1134.

Klein, R., et al. (1995), Ophthalmol., 102(3), 371-81.

Kliffen, M. et al. (1995), Lab. Invest., 73(2), 267-72.

Kliffen, M., et al. (1994), Invest. Ophthalmol. Vis. Sci., 35, 2901-2905.

Kliffen, M., et al. (1996), Arch. Ophthalmol., 114, 1009-14.

Kliffen, M., et al. (1997), Microscopy Res. Tech., 36(2), 106-22.

Kligman, A. (1969), JAMA, 210, 2377-2380.

Kohler and Milstein (1975), Nature, 256, 495-497.

Komher et al. (1989), Nucl. Acids. Res., 17,7779-7784.

Kozbar et al. (1983), Immunol. Today, 4, 72.

Krol et al. (1988), BioTechniques, 6, 958-976.

Kuppuswamy et al. (1991), Proc. Natl. Acad. Sci. USA, 88, 1143-1147.

Kwoh et al. (1989), Proc. Natl. Acad. Sci. USA, 86, 1173-1177.

Laemmli, U. (1970), Nature, 227, 680-685.

Lakso et al. (1992), Proc. Natl. Acad. Sci. USA, 89, 6232-6236.

Lamand, A., and Mann, M. (1997), Trends in Cell Biol., 7, 139-142.

Landegran et al. (1988), Science, 241, 1077-1080.

LaVail et al. (1998), Invest. Ophthalmol. Vis. Sci., 39, 592-602.

La Vail et al. (1998), Invest. Ophthalmol. Vis. Sci., 39, 581-591.

Lazarus, H., et al. (1993), Exp. Eye Res., 56, 531-541.

Leibowitz, H., et al. (1980), Survey of Ophthalmol., 24, 428-457.

Lemaitre et al. (1987), Proc. Natl. Acad. Sci. USA, 84, 648-652.

Letsinger et al. (1989), Proc. Natl. Acad. Sci. USA, 86,6553-6556.

Lewis, H., et al. (1986), Ophthalmol., 93, 1098-1112.

Leys, A., et al. (1990), Graefe's Arch. Clin. Exp. Ophthalmol., 228, 499-504.

Leys, A., et al. (1991), Graefe's Arch. Clin. Exp. Ophthalmol., 229, 406-10.

Li, J., and McAdam, W. (1984), Scand. J. of Immunol., 20, 219-226.

Li, X., et al. (1995), Arterioscler., Thromb., and Vasc. Bio., 15, 252-257.

Liem, A., et al. (1992), Amer. J. Ophthalmol., 114, 149-157.

Lim, J., et al. (1993), Retina, 13, 230-3.

Lizardi et al. (1988), Bio/Technology, 6, 1197.

Loeffler, K., and Mangini, M. (1997), Graefe's Arch. Clin. Exp. Ophthalmol., 235, 248-54.

Loeffler, K., et al.(1995), Invest. Ophthalmol. Vis. Sci., 36, 24-31.

Lovie-Kitchin, J., and Bowman, K. (1985), Senile Macular Degeneration. Boston: Butterworth.

Luna, L. (Ed.). (1968), Manual of Histological Staining Methods of the Armed Forces Institute of Pathology (3rd ed.). New York: McGraw-Hill.

Macular Photocoagulation Study. (1997), Arch. Ophthalmol., 115(6), 741-7.

Maggio (ed.) (1980), Enzyme Immunoassay, CRC Press, Boca Raton.

Maher. (1992), Bioassays, 14, 807-815.

Mansergh, F., et al. (1995), J. Med. Genet., 32, 855-8.

Mares-Perlman, J., et al. (1995a), Arch. Ophthalmol., 113, 1518-23.

Mares-Perlman, J., et al. (1995b), Arch. Ophthalmol., 113, 743-8.

Marmor, M., and Byers, B. (1977), Amer. J. Ophthalmol., 84, 32-44.

Massof, R., et al. (1989), Clin. Vision Sci., 4, 221-227.

Maxim and Gilbert (1977), Proc. Natl. Acad. Sci. USA, 74, 560.

Mayer and Walker, (eds.) (1987) Immunochemical Methods In Cell And Molecular Biology, Academic Press, London.

Mehregan, A., and Hashimoto, K. (1991), Pinkus' Guide to Dermatohistopathology (pp. 369-382). Englewood Cliffs, N.J.: Prentice Hall.

Meyers, S. (1994), Transact. Amer. Ophthalmol. Soc., 92, 775-843.

Meyers, S., and Zacchary, A. (1988), Arch. Ophthalmol., 106, 651-653.

Meyers, S., et al. (1995), Amer. J. Ophthalmol., 120, 757-66.

Midena, E., et al. (1997), Invest. Ophthalmol. Vis. Sci., 38, 469-477.

Midena, E., et al. (1994), Documenta Ophthalmologica, 88, 179-85.

J. H. Miller and M. P. Calos, (eds.) (1987) Gene Transfer Vectors For Mammalian Cells, Cold Spring Harbor Laboratory.

Mimoun, G., et al. (1990), J. Fr. Ophtalmol., 13(10), 511-30.

Monaco, W., and Wormington, C. (1990), Optomet. Vis. Sci., 67, 532-537.

Moore, D., et al. (1995), Invest. Ophthalmol. Vis. Sci., 36, 1290-1297.

Muda, A., et al. (1988), Virchows Archiv—A, Path. Anat. Histopath., 413, 529-37.

Muller, H. (1856), Albrecht von Graefe Archiv fur Ophthalmologie, 2(2), 1-69.

Mullins, R., and Hageman, G. (1997), Histochemical comparison of ocular “drusen” in monkey and human. In M. LaVail, J. Hollyfield, and R. Anderson (Eds.), Degenerative Retinal Diseases (pp. 1-10). New York: Plenum Press.

Mullins, R., et al. (1997a), Ophthalmol., 104, 288-294.

Mullins, R. et al. (1999), FASEB J. 13:X-X.

Myers, et al. (1985), Science, 230, 1242.

Myers et al. (1985), Nature, 313, 495.

Nakazawa et al. (1994), Proc. Natl. Acad. Sci. USA, 91, 360-364.

Newsome, D. (1988), Angioid streaks and Bruch's membrane degenerations. In D. Newsome (Ed.), Retinal Dystrophies and Degenerations (pp. 271-283). New York: Raven Press.

Newsome, D., et al. (1987), Amer. J. Ophthalmol., 104, 373-381.

Newsome, D., et al. (1987b), Curr. Eye Res., 6, 1211-1221.

Newsome, D., et al. (1986), Invest. Ophthalmol. Vis. Sci., 27, 1675-80.

Nickerson et al. (1990), Proc. Natl. Acad. Sci. USA, 87, 8923-8927.

Niculescu, F., et al. (1987), Atherosclerosis, 65, 1-11.

Niculescu, F., et al. (1989), Atherosclerosis, 78, 197-203.

Nyren et al. (1993), Anal. Biochem., 208, 171-15.

O'Gorman et al. (1991), Science, 251, 1351-1355.

O'Shea, J. G. (1996), Med. J. Austr., 165(10), 561-4.

Olin, K., et al. (1995), Proc. Soc. Exp. Biol. Med., 208, 370-377.

Oppenheim, J., and Leonard, E. (1989), Introduction to human monocytes. In M. Zembala and G. Asherson (Eds.), Human Monocytes San Diego, Calif.: Academic Press.

Orban et al. (1992), Proc. Natl. Acad. Sci. USA, 89, 6861-6865.

Orita et al. (1989), Proc. Natl. Acad. Sci. USA, 86, 2766.

Osusky, R., et al. (1997), Recep. Sign. Trans., 5, 43-50.

Parodi, M. (1994), Acta Ophthalmologica, 72, 260-4.

Pauleikhoff, D., et al. (1990), Amer. J. Ophthalmol., 109, 38-43.

Pauleikhoff, D., et al. (1990b), Ophthalmol., 97, 171-178.

Pauleikhoff, D., et al. (1994)[German], Ophthalmologe, 91(6), 730-4.

Pauleikhoff, D., et al. (1992), Ophthalmol., 99, 1548-1553.

Penfold, P., et al. (1985), Graefe's Arch. Clin. Exp. Ophthalmol., 223, 69-76.

Penfold, P., et al. (1986), Invest. Ophthalmol. Vis. Sci., 27, 364-71.

Penfold, P., et al. (1990), Graefe's Arch. Clin. Exp. Ophthalmol., 228, 270-4.

Perbal (1984), A Practical Guide To Molecular Cloning Methods In Enzymology (Academic Press, Inc., N.Y.).

Perry-O'Keefe et al. (1996), Proc. Natl. Acad. Sci. USA, 93, 14670-14675.

Piguet, B., et al. (1995), Eye, 9, 34-41.

Piguet, B., et al. (1996), Ophthalmic Gen., 17(4), 175-86.

Piguet, B., et al. (1993), Br. J. Ophthalmol., 77, 400-403.

Preissner, K. (1991), Ann. Rev. Cell. Biol., 7, 275-310.

Prezant et al. (1992),. Hum. Mutat., 1, 159-164.

Prossner (1993), Tibtech, 11, 238.

Puchtler, H., et al. (1962), J. Histochem. Cytochem., 10, 355-364.

Reblin, T., et al. (1995), Atherosclerosis, 113, 179-88.

Rekhter, M., et al. (1993), Amer. J. Pathol., 143, 1634-1638.

Ritter, L., et al. (1995), Amer. J. Ophthalmol. 120(2), 190-6.

Robertson et al. (1986), Nature, 322, 445-448.

Robertson (1987) in: Teratocarcinomas and Embryonic Stem Cells: A Practical Approach, E. J. Robertson, ed., IRL Press, Washington, D.C.

Roest, et. al. (1993), Hum. Mol. Genet., 2, 1719-21.

Roitt, I., et al. (1985), Immunol., St. Louis: C. V. Mosby.

Rones, B. (1937), Formation of drusen of the lamina vitrea.

Rosenbaum and Reissner (1987), Biophys. Chem., 265, 12753.

Rosenfeld, M., et al. (1993), Arterioscl. Thromb., 13, 1382-9.

Rudnew, A. (1871), Archiv fur Pathologische Anatomie und Physiologie und fur Klinische Medicin, 53, 455-465.

Rus, H., et al. (1989), Immunol. Lett., 20, 305-10.

Saiki et al. (1986), Nature, 324, 163.

Saiki et al (1989), Proc. Natl. Acad. Sci. USA, 86, 6230.

Saitta, M., et al. (1992), Clin. Chem., 38, 2454-7.

Salib, E., and Hillier, V. (1997), Internat. J. Ger. Psych., 12(3), 363-8.

Saleeba et al. (1992), Meth. Enzymol., 217, 286-295.

Sambrook, Fritsch and Maniatis, (eds.), (1989) Molecular Cloning A Laboratory Manual, 2nd Ed., ed. by Cold Spring Harbor Laboratory Press, Chapters 16 and 17.

Sandberg, M., et al. (1994), Invest. Ophthalmol. Vis. Sci., 35(6), 2734-40.

Sanger et al (1977), Proc. Nat. Acad. Sci. USA, 74, 5463

Sarin et al. (1988), Proc. Natl. Acad. Sci. USA, 85, 7448-7451.

Sarks, J., et al. (1988), Eye, 2, 552-577.

Sarks, J., et al.(1994), Eye, 8, 269-283.

Sarks, S. (1976), Br. J. Ophthalrnol., 60, 324-341.

Sarks, S. (1980), Aust. J. Ophthalmol., 8, 117-30.

Sarks, S. (1982), Austr. J. Ophthalmol., 10, 91-97.

Sarks, S., and Sarks, J. (1989), Age-related macular degeneration: atrophic form. In S. Ryan (Ed.), The Retina St. Louis: C V Mosby.

Sarks, S., et al. (1985), Pattems in macular-degeneration. In S. Ryan, A. Dawson, and H. Little (Eds.), Retinal Diseases (pp. 87-93). Orlando: Grune and Stratton.

Sarks, S., et al. (1980), Trans. Ophthalmol. Soc. U.K., 100, 414-422.

Sarver et al. (1990), Science, 247, 1222-1225.

Schachat, A., et al. (1995), Arch. Ophthalmol., 113(6), 728-35.

Schalch, W. (1992), Exs, 62, 280-98.

Seddon, J., et al. (1997), Amer. J. Ophthalmol., 123, 199-206.

Seddon, J., et al. (1994), JAMA, 272(18), 1413-20.

Seiffert, D., et al. (1994), J Biol Chem, 269, 19836-19842.

Sheraidah, G., et al. (1993), Ophthalmol., 100, 47-51.

Sigelman, J. (1991), Ophthalmol.,98, 1379-1383.

Silvestri, G., et al. (1994), Eye, 8, 564-8

Small, K., et al. (1993), Ophthal. Paediat. Genet., 14(4), 143-150.

Small, M., et al. (1976), Arch. Ophthalmol., 94, 601-607.

Smithies et al. (1985), Nature, 317, 230-234.

Sokolov (1990), Nucl. Acids Res., 18, 3671.

Solem, M., et al. (1991), Mol. Cell. Biochem., 100, 141-9.

Spraul, C., and Grossniklaus, H. (1997), Arch. Ophthalmol., 115, 267-73.

Stafford, T., et al. (1984), Ophthalmol., 91, 513-521.

Stangos, N., et al. (1995), Ophthalmologica, 209, 194-8.

Starita, C., et al. (1996), Exp. Eye Res., 62, 565-572.

Stary, H., et al. (1995), Circulation, 92, 1355-1374.

Steinmetz, R., et al. (1993), Br. J. Ophthalmol., 77, 549-554.

Stewart et al. (1987), EMBO J., 6, 383-388.

Stockmann, A., et al. (1993), J. Biol. Chem., 268, 22874-82.

Stone, E. et al. (1998) Nature Med. December

Stone, E., and Sheffield, V. (1993), Moleclar Genetics of Inherited Eye Disorders. Chur, Switzerland: Academic Publishers.

Stone, E., et al. (1992), Nature Genet., 1, 246-250.

Stone, K., et al. (1990), Meth. Enzymol., 193, 389-412.

Strittmatter, W. J., and Roses, A. D. (1995), Proc. Nat. Acad.Sci. USA, 92(11), 4725-7.

Sunness, J., et al. (1988), Arch. Ophthalmol., 106, 1081-1084.

Sunness, J., et al. (1989), Ophthalmol., 96, 375-381.

Sunness, J., et al. (1985), Arch. Ophthalmol., 103, 811-816.

Syvanen, et al. (1990), Genomics, 8, 684-692.

Syvanen et al. (1993), Amer. J. Hum. Genet., 52, 46-59.

Tabatay, C., et al. (1987), Arch. Ophthalmol., 105, 826-830.

Tan, S., and Pepys, M. (1994), Histopathol., 25, 403-414.

Tanzi, R. E., et al. (1996), Neurobiol. Dis., 3(3), 159-68.

Taylor, H. R., et al. (1990), Trans. Amer. Ophthalmol. Soc., 88, 163-73.

Territo, M. C., and Cline, M. J. (1975), Clin. Haematol., 4(3), 685-703.

Thomas & Capecchi (1987), Cell, 51, 503-512.

Thompson et al. (1989), Cell 5, 313-32.Thorner, P., and Baumal, R. (1982), Arch. Path. Lab. Med., 106, 628-31.

Tobe et al. (1996), Nucleic Acids Res., 24, 3728.

Tolentino, M., et al. (1994), Vis. Res., 34(3), 409-13.

Tomasini, B., and Mosher, D. (1991), Prog. Hemost. and Thromb., 10, 269-305.

Ugozzoli et al. (1992), GATA, 9, 107-112.

Ulshafer, R., et al. (1987), Retina, 7, 198-203.

Umemoto, J., et al. (1977), J. Biol. Chem., 252, 8609-14.

Vachino, G., et al. (1988), J. Leuk. Biol., 44, 529-34.

van der Luijt, et. al. (1994), Genomics, 20, 1-4.

Van der Putten et al. (1985), Proc. Natl. Acad. Sci. USA, 82, 6148-6152.

van der Schaft, T., et al. (1993), Br. J. Ophthalmol., 77, 657-661.

van der Schaft, T., et al. (1994), Graefe's Arch. Clin. Exp. Ophthalmol., 232, 40-46.

van der Schaft, T., et al. (1992), Ophthalmol., 99, 278-86.

Vinding, T. (1 990), Acta Opthalmologica, 68, 410-414.

Vingerling, J., et al. (1995), Amer. J. Epidem., 142, 404-9.

Vingerling, J., et al. (1996), Arch. Ophthalmol., 114, 1193-1196.

Volker, W., et al. (1993), J. Histochem. Cytochem., 41, 1823-1832.

Voller (1 978), Diagnostic Horizons, 2, 1-7.

Voller et al. (1978), J. Clin. Pathol., 31, 507-520.

Wagner et al. (1981), Proc. Natl. Acad. Sci. USA, 78, 1441-1445.

Wallow, I., and Tso, M. (1972), Amer. J. Ophthalmol., 73, 914-926.

Weintraub, B., (1986) Principles of Radioimmunoassays, Seventh Training Course on Radioligand Assay Techniques, The Endocrine Society.

Weir, D. M. and Blackwell, C. C. (eds.) (1986), Handbook Of Experimental Immunology, Volumes I-IV.

Wetzig, P. (1988), Trans. Am. Ophthalmol. Soc., 86, 276-290.

Williams, K., et al. (1996), Matrix assisted-laser desorption ionization mass spectrometry as a complement to internal amino acid sequencing. In J. Walker (Ed.), The protein protocols handbook (pp. 541-555). Totowa: Humana Press.

Wolter, J., and Falls, H. (1962), Arch. Ophthalmol., 68, 219-226.

Wu et al., (eds.) Methods In Enzymology, Vols. 154 and 155, Academic Press Inc., N.Y;

Yamamoto et al. (1980), Cell, 22, 787-797.

Yang, G., et al. (1992), Am. J. Path., 141, 409-419.

Yanoff, M., and Fine, B. (Date), Ocular Pathology. A Color Atlas. New York: Gower Medical Publishing.

Yasumitsu, H., et al. (1993), In vitro Cell. Devel. Biol., 29A, 403-407.

Young, R. (1987), Surv. Ophthalmol., 31, 291-306.

Young, R. W. (1988), Surv. Ophthalmol., 32(4), 252-69.

Zahedi, K. and Whitehead, A. S. (1989), J. immunol., 143,2880-2886.

Zaug et al. (1984), Science, 224, 574-578.

Zaug and Cech (1986), Science, 231, 470-475.

Zaug et al. (1986), Nature, 324, 429-433.

Zeller, C., and Marchase, R. (1992), Am. J. Physiol., 262, C1341-C1355.

Zon (1988), Pharm. Res., 5, 539-549.

### EXAMPLES

### Example 1

### Human Donor Eve Repository and “Comprehensive Donor Database” (CDD)

Tissues from the a unique human donor eye repository and comprehensive donor database (CDD) have been employed for the experiments described in the Examples that follow.

This research employs a human donor eye repository that has been developed over the past eight years. The repository contains over 2,000 pairs of eyes. Staff are on 24 hour call to retrieve and process donated tissue. Eyes are accepted only if they can be processed within four hours of death. A database of clinical, statistical and scientific information for each donor eye entered into the repository has been developed and will continue to be maintained. Sera, blood (DNA), ophthalmologic and medical histories, and family interviews are collected for as many donors as possible; these data have been collected for over 90% of the donors entered into the repository in the past two years. Over 25% of our donors in the last two years have a clinically documented history of AMD.

A standard procedure for processing eyes has been developed; this procedure is modified, when required, to meet the needs of specific studies. All eyes are photographed immediately. Every eye is processed similarly such that reproducible regions are available for comparative biochemical, molecular and morphological analyses. Briefly, the posterior pole is pinned to a wax plate following the placement of four incisions directed towards, but not passing through, the macula. Grossly, eyes (or gross photographs) are examined under a dissecting microscope at 6-10×. Subjective grading of drusen size, density and class is recorded. Macular drusen are classified into the following categories: rare (<5 drusen), few (6-50 drusen), moderate (51-200 drusen), and numerous (>200-300); and sizes: small (<50 μm), moderate (50-500 μm), and large (>50 μm). Additional features of age-related macular degeneration, such as macular increased RPE pigmentation, macula RPE pigment clumping, RPE atrophy, subretinal or sub RPE hemorrhage are also noted. In general, “early” AMD is defined as 1) the presence of indistinct (“soft”) or reticular drusen, or 2) presence of any drusen type with associated visual loss, RPE degeneration, and/or abnormal retinal pigment in the macular area. “Late” AMD is defined as the presence of exudative AMD (RPE detachment, detachment of the retina, subretinal or subRPE hemorrhage, or subretinal fibrous scars) or geographic atrophy. At this stage, eyes are graded based upon an adaptation of a classification system developed by The International ARM Epidemiological Study Group; this information is entered into a database containing all information available for each donor.

Following gross examination, the vitreous is removed and various regions excised with trephine punches; these are frozen immediately in liquid nitrogen or fixed as per the protocol. The neural retina is separated from the RPE/choroid in regions that are punched. Portions of every eye are processed for light and electron microscopic analyses. Wedges composed of equatorial/peripheral retina are removed with forceps and frozen similarly. Sections made from all eyes are stained with hematoxylin and eosin, Mallory trichrome, PAS, oil red O, and Sudan black B. Histopathologic and electron microscopic examination of all donor eyes, that includes portions of the maculas from most eyes, is performed. Based on these analyses, drusen are classified into distinct morphologic phenotypes. These categories resemble most closely the classification scheme proposed by Sarks. It is from these morphological analyses that eyes are divided into experimental groups for the proposed biochemical and molecular studies.

We have also developed the “Comprehensive Donor Database” (CDD), a rigorously characterized group of donors of all ages, with and without AMD. To date, we have placed 231 donors from our repository into the CDD, ranging in age from 1 day to 101 years. 31% of these donors had a clinically documented history AMD. The CDD is comprised of at least donors per decade up to 50 years. Decades above the age of 50 years are comprised of 15 donors each with clinically documented AMD (5 each with macular drusen, geographic atrophy, and choroidal neovascular membranes or disciform scars) and 10 age-matched controls.

Fixed (4% paraformaldehyde; Kamovsky) and frozen tissue is available for all donors (see Human Subjects section). In addition, histologic sections of all the eyes that have been entered to date have been made. Sections stained with H&E, PAS, Oil Red 0, and Sudan black B, and prepared for the examination of autofluorescence, are available for every eye entered into the CDD. Approximately 20% of the eyes thus far have been examined by electron microscopy. Micrographs from both the maculas and peripheral regions have been recorded at standardized magnifications. Baseline morphometric data from each eye are being obtained. These include measurements of drusen size, number, and phenotype; BLD density and distribution; RPE and photoreceptor cell densities; Bruch's membrane thickness and degree of debris accumulation; choriocapillaris density; and choroidal thickness and density of choroidal fibrils. Other parameters will be added as required.

### Example 2

### Identification of Distinct Core Subdomains within Drusen

Reagents: Fluorescein isothiocyanate- (FITC-) and rhodamine-conjugated lectins derived from Limax flavus (LFA), Triticum vulgaris (WGA), Arachea hypogea (PNA), and Ricinis commnunis (RCA-1) were obtained from EY Laboratories, Inc. (San Mateo, Calif.) and Vector Laboratories (Burlingame, Calif.). Neuraminidase (isolated from Clostridium perfringens) was obtained from Boehringer-Mannheim (Indianapolis, Ind.) and O-glycanase (endo-a-N-acetylgalactosaminidase) was purchased from Genzyme (Cambridge, Mass.) and Boehringer-Mannheim (Indianapolis, Ind.). Sudan black B solution was obtained from Poly Scientific (Bay Shore, N.Y.), and PNGase F and globulin free bovine serum albumin (BSA) were purchased from Sigma Chemical Company (St. Louis, Mo.). Immumount was purchased from Shandon (Pittsburgh, Pa.). Acrylamide and other reagents used for embedding were purchased from Bethesda Research Laboratories (Bethesda, Md.). Optimal Cutting Temperature compound (OCT) was obtained from Miles Inc. (Elkhart, N.Y.). Superfrost Plus slides were obtained from Fisher (Pittsburgh, Pa.). Materials for transmission electron microscopy were obtained from Fluka Chemika-BioChemika (Ronkonkoma, N.Y.).

Human Donor Eyes: Eyes from 42 human donors, ranging from 35 to 101 years of age, were obtained from MidAmerica Transplant Services (St. Louis, Mo.) and the Iowa Lions Eye Bank (Iowa City, Iowa). Eyecups were preserved in 4% paraformaldehyde in 100 mM sodium cacodylate, pH 7.4, or embedded directly in Optimal Cutting Temperature compound (OCT) and frozen in liquid nitrogen, without fixation, within six hours post-mortem.

Fixation and Embedding: After 2-4 hrs in fixative, eyecups were placed into buffer and embedded, as described in the previous Example.

Glycosidase Treatments: Sections of fixed and unfixed tissues were incubated with 1 U/mL neuraminidase in 30 mM sodium acetate buffer, pH 5, at 37° C., overnight in a humidified chamber. Sections from 41 eyes—33 fixed and acrylamide-embedded and 9 unfixed and OCT-embedded—were treated with neuraminidase and subsequently labeled with FITC- or rhodamine-PNA (below). Adjacent, control sections were incubated with buffer alone. Serial sections from two eyes (one fixed and one unfixed) were treated with neuraminidase and labeled with LFA, RCA-I, ConA, and WGA, to determine the effects of neuraminidase on labeling of drusen with other lectins. Tissue sections from two additional donors were pretreated with neuraminidase and subsequently treated with O-glycanase (1 U/mL in 15 mM sodium cacodylate, pH 6) or PNGase F (1 U/mL in PBS), for 72 hours, at 37° C. Control sections were treated with buffer alone.

Lectin Histochemistrv: For lectin labeling, 6-8 mm thick cryostat sections were cut, mounted on Superfrost Plus slides, and labeled with PNA, WGA, or LFA as described above. Unlabeled, adjacent control sections were used to distinguish between lectin binding and drusen autofluorescence.

In order to compare labeling ofenzyme- and buffer-treated sections, intensity and binding patterns on serial sections containing the same drusen were compared. Identical exposure times for experimental pairs were used during photomicrography and during photographic processing.

Transmission Electron Microscopy: Drusen-containing tissues were obtained as above, and were fixed within 4 hours of death in one half-strength Kamovsky's fixative and processed as described below. Reagents employed in embedding tissues for transmission electron microscopy were obtained from Fluka Chemical (Milwaukee, Wis.). All other reagents were obtained from Electron Microscopy Sciences (Fort Wayne, Pa.). Tissues were fixed for at least two hours in one-half strength Kamovsky fixative (1/2K; 2% formaldehyde and 2.5% glutaraldehyde in lOOmM cacodylate buffer, pH 7.4, containing 0.025% CaCl2) prior to washing 3×10 min. in 100 mM cacodylate buffer. Pellets or wedges were then post fixed with 2% osmium tetraoxide in cacodylate buffer for 2 hours, and were then rewashed 3×10 min. prior to dehydration through a series of graded ethanol solutions (50% ethanol 10 min. each in 70% ethanol, and 95% ethanol, followed by 2×10 min. in 100% ethanol. Tissues were then dehydrated 2×10 min. in propylene oxide, and were infiltrated overnight in a 1:1 mixture of propylene oxide:Epon 812 solution (containing 51% Epon 812, 27% dodecenyl succinic anhydride, and 22% nadic methyl anhydride, with 1.5% DMP-30 added to the solution as an accelerator). The following day the Epon solution was changed 3 times throughout the day, and the samples were cured at 40%C overnight and then at 65%C for 2 days.

Thin sections (60-75nm) were taken from Epon-embedded tissues, on a Reichert-Jung Ultracut ultramicrotome. Sections were collected on nickel grids and were stained with 2% aqueous uranyl acetate and Reynold's lead citrate.

Results

Neuraminidase Treatment: Incubation of drusen-containing tissue sections with neuraminidase completely eliminated LFA labeling of drusen and other structures in the chorioretinal complex, as compared to controls. Labeling of nuclei in the choroid and neural retina persisted after neuraminidase treatment, however. This loss of labeling was used throughout the study to control for enzyme efficacy, as were changes in labeling of the interphotoreceptor matrix, as described previously (Johnson and Hageman, 1987; Kivela, 1990).

Labeling of drusen with WGA, RCA-I, and Con A is not significantly diminished following neuraminidase treatment, demonstrating that the binding of these lectins to drusen-associated glycoconjugates is not primarily due to sialic acid. In some eyes, the intensity of WGA labeling of the choroidal stroma decreases after neuraminidase treatment, without a concomitant loss of WGA binding to drusen in the same section.

PNA does not normally bind to drusen or any other structure within the RPE-choroid complex. Following pre-exposure to neuraminidase, the endothelial cells and/or endothelial cell basal laminae of the choriocapillaris and other choroidal vessels, as well as the basal lamina of the RPE, bound PNA intensely.

Some drusen were labeled by PNA following exposure to neuraminidase. This intense labeling was typically restricted to subdomains, or “cores”, within drusen. These cores were not observed in adjacent sections treated with buffer alone. Drusen cores were typically spherical, centrally located within the drusen, and juxtaposed against the inner collagenous layer of Bruch's membrane. Only one core was generally observed within any given solitary drusen, whereas confluent or fused drusen may possess several cores. These cores ranged from 5 to 30 μm diameter, with a mean diameter of 14 μm.

Drusen cores were observed frequently; they were present in 32 of the 42 eyes examined in this study. No differences in the appearance or frequency of these deposits was noted with respect to fixation and embedding conditions. Intense labeling of drusen cores was observed using both FITC-PNA and rhodamine-conjugated PNA, demonstrating that this observation is not due to interaction with the fluorophore. In addition, both hard and soft drusen possessed cores, although large, soft drusen typically had PNA-binding cores which are larger and less centrally localized than those of hard drusen.

Enzymatic Characterization of Drusen Cores: Following incubation with O-glycanase, PNA labeling of the choroid and interphotoreceptor matrix was nearly or completely abrogated. Labeling of drusen cores with PNA was significantly reduced following O-glycanase treatment. O-glycanase treatment had little effect on labeling of rod outer segments with ConA, suggesting that contaminating N-glycosidase activity was not present. In contrast to O-glycanase treatment, PNGase F pretreatment did not change the intensity of PNA labeling of drusen cores. ConA labeling of rod outer segments was completely abrogated following incubation with PNGase F, demonstrating that the enzyme efficiently removed N-linked glycans.

When PNA-labeled or unlabeled tissue sections were stained with Sudan black B, core-like regions were not stained. Serial sections, stained alternatively with PNA (following neuraminidase pretreatment) and Sudan black B revealed that the PNA-positive cores and Sudan black-negative cores colocalize.

Transmission Electron Microscopy: Transmission electron microscopy revealed a variety of possible “core-like” structures within drusen. These include regions which exhibited subtle differences in contrast due to differences in quantity and/or electron density of particles, when compared to the rest of the drusen, as well as domains which were very dense and osmiophilic. In addition, drusen occasionally possessed regions that are electron lucent, presumably due to extraction of lipids during processing.

### Example 3

### Dendritic Cells and Proteins Involved in Immune-Mediated Processes are Associated with Drusen and Drusen Biogenesis

Introduction: Drusen are a significant risk factor for the development of age-related macular degeneration (AMD). Relatively little is known, however, about their origin(s). We recently described the presence of centralized domains comprised of distinct saccharides within drusen (J Histochem Cytochem 47;1533-9, 1999). Electron microscopic analyses have revealed that cell processes, derived from choroidal cells, breach Bruch's membrane and terminate in bulbous cores within drusen.

Reagents: All supplies for electron microscopy were obtained from Fluka Chemical (Milwaukee, Wis.). Antibodies to CD3, CD15, CD45, and anti-mouse secondary antibodies conjugated to indocarbocyanine-3 (Cy3) were purchased from Chemicon International (Temecula, Calif.); monoclonal antibodies to CD1a, CD 14, CD31, CD45, CD68, S100 and HLA-DR were purchased from Dako (Carpenteria, Calif.). Fluorescein-conjugated secondary antibodies were obtained from Jackson Immunoresearch Laboratories (West Grove, Pa.). For some experiments, the Elite Staining Kit was employed and labeling was visualized with the Vector VIP substrate (Vector, Burlingame, Calif.). Neuraminidase (Clostridium perfiingens) was obtained from Boehringer-Mannheim (Indianapolis, Ind.). Fluorescein-conjugated peanut agglutinin was purchased from EY Laboratories, Inc. (San Mateo, Calif.). Other reagents used for tissue fixation and embedment were obtained from Sigma Chemical Company (St. Louis, Mo.), unless otherwise noted. Studies were conducted to immunophenotype the choroidal cells from which these core terminations arise and to evaluate their potential relationship to drusen biogenesis.

Tissues: Human donor eyes employed in this study were obtained from The University of Iowa Lions Eye Bank (Iowa City, Iowa) within four hours of death. Institutional Review Board committee approval for the use of human donor tissues was obtained from the Human Subjects Committee at The University of Iowa. Posterior poles, or wedges of posterior poles spanning between the ora serrata and macula, were processed from 30 donors, embedded in OCT, snap frozen in liquid nitrogen, and stored at −80° C. Tissues were sectioned to a thickness of 6-8 μm on a cryostat. Confocal laser scanning microscopy and immunohistochemistry were employed to examine drusen-associated cores in human donor eyes. Immunolabeling of sections was performed using a battery of antibodies directed against various cell-populations including endothelial-cells, lymphocytes, granulocytes, monocytes/macrophages and dendritic cells.

Transmission Electron Microscopy: For transmission electron microscopy, posterior poles were fixed in one-half strength Karnovsky's fixative (see Example 1) within 4 hours of death. Macular punches and saggittal wedges from over 200 eyes obtained by our laboratory were fixed and processed for electron microscopy, as described previously (Lazarus, et al., 1993), and used for these analyses.

Immunohistochemistry: Eyes employed for immunohistochemistry were dissected and embedded into Optimal Cutting Temperature compound (Miles Inc.; Elkhart, N.Y.) without prior fixation. Cryostat sections were cut at a thickness of 6-8 mm and were collected on Superfrost plus slides (Fisher; Pittsburgh, Pa.). Neuraminidase treatment was performed in some cases; sections were digested overnight with IU/niL neuraminidase in 30 mM sodium acetate buffer, pH 5.0, overnight at 37° C. For fluorescence microscopy, antibody and lectin labeling was performed as described previously in Example 1. For some experiments, the Vector Elite kit for horseradish peroxidase staining was used, according to the manufacturer's instructions. In order to determine whether leukocyte antigens and PNA-binding cores colocalize, serial sections were incubated alternately with PNA (neuraminidase pretreated) and anti-CD antibodies. Control sections were treated with secondary antibodies alone. Positive controls for CD antibodies were based on their reactivity with leukocytes in the choroidal vasculature and stroma. Reactivity of drusen with antibodies to HLA-DR and CD68 was quantitated in unfixed sections by calculating the percentage of labeled drusen. The diameters of drusen and of drusen cores were measured with an eyepiece reticle calibrated to a stage micrometer.

Double Labeling/Confocal Microscopy: Cryostat sections were treated overnight with neuraminidase (above) followed by immunolabeling with a monoclonal antibodies directed against CD68 HLA-DR, and CDla. Alexa 488-or Cy-3-conjugated secondary antibodies (Chemicon; Temecula, Calif.: Molecular Probes; Eugene, Oreg.) were used to visualize CD68 immunoreactivity. Sections were washed extensively and incubated with PNA conjugated to fluorescein. Confocal images of both probes were collected simultaneously using a confocal microscope (BioRad; Hercules, Calif.). Positive controls included choroidal and scleral leukocytes.

Results: Significantly, cellular processes, derived from cells in the ohoroidal stroma, were observed that breach Bruch's membrane and terminate within drusen. Extensive serial sectioning through five drusen revealed a single process, derived from a choroidal cell, passing through Bruch's membrane and-terminating as a bulbous process, occupying the same location as drusen cores. The choroidal cells from which these processes emanate exhibited a large degree of rough endoplasmic reticulum, had nuclei that are lobed, and were dendritic in shape. No large granules or lysosomes were apparent in their cytoplasm. These processes were also observed lying adjacent to whole, or portions of, RPE cells, in regions without significant numbers of drusen.

In order to examine the association of these choroidal cell-derived processes to drusen cores, cryostat sections were incubated with antibodies to CD45 (leukocyte common antigen), while alternate serial sections were digested with neuraminidase and labeled with PNA. A subset of the same cores which bound PNA are also labeled with CD45 antibodies. Anti-CD45 antibodies colocalize with PNA-binding cores in smaller drusen.

As detailed in Table 2, drusen cores, and the cells from which they are derived, are also strongly reactive with CD1a, CD4, CD11a, CD11c, CD14, CD18, CD31, CD40, CD45, CD64, CD68, CD83, CD86, and HLA class II (CR3/43 and TAL.1B5) antibodies. The CR3/43 antibody reacts with MHC class II antigens including HLA-DP, HLA-DQ, and HLA-DR, whereas the TAL.1B5 antibody is specific to HLA-DR alpha chains. Both antibodies react with drusen cores, although the DR-specific clone (TAL.1B5) may react with more restricted, cell-associated domains whereas the pan-MHC-II clone (CR3/43) may label more voluminous domains within drusen. This may imply that HLA-DR is largely confined to drusen-associated dendritic cells, in contrast to HLA-DP and HLA-DQ, which may be derivatives of membrane blebs from these cells, or exosomes. Ongoing studies are be directed toward determining whether there is a difference in the distribution of the various MHC class-II antigens in drusen cores, and whether other exosomal proteins are present in drusen.

To gain further insight into the relationship between the distribution of leukocyte processes and drusen, double-labeled tissue sections were examined using scanning laser confocal microscopy. Two relevant observations were made. In some drusen, there was a direct colocalization of PNA and anti-CD68 antibody to the drusen cores. However, CD68, but not PNA, labeled the body of the choroidal cell associated with the core. These observations suggest that the core-associated PNA-binding material was restricted to the bulbous cell process or that it was secreted by these processes into drusen. In other cases, a small CD68-immunoreactive core was observed that is surrounded by a larger, PNA-binding cuff, consistent with the proposition that this material was synthesized and secreted by core-associated macrophages/dendritic cells, and/or that the bulbous processes modify the surrounding drusen-associated matrix such that it bound PNA.

Quantitative studies indicate that these drusen-associated cores are present in approximately 40% of drusen. Drusen cores appear to be more prevalent in smaller drusen, and are also detected as putative drusen precursors, solitary cores within Bruch's membrane that are not surrounded by additional drusenoid accretions.

The number of HLA-DR and CD68 immunoreactive drusen were determined in unfixed cryostat sections. Eighty-eight percent of all drusen were HLA-DR immunoreactive; binding was restricted to cores in some drusen, whereas in others it was observed throughout.

The mean size of HLA-DR reactive drusen was 26 μm+9 μm. The mean size for HLA-DR negative drusen was 22.8 μm+4.81 μm. Thus, there was no significant difference in size between HLA-DR positive and negative drusen (Student's t-test). In contrast, approximately twenty percent of all drusen in any given eye possessed anti-CD68 antibody immunoreactive cores.

The diameters of cores that reacted with antibodies to CD1a, CD45 and CD68 were measured with an eyepiece reticle. These cores measured 10.4 μm+4.4 μm in diameter. This was somewhat smaller than the average size of PNA-binding cores (14 μm), and may suggest that the PNA-binding material in drusen surrounds the leukocyte process. This result is consistent with results from double labeling confocal microscopy experiments (above).

Conclusions: The immunophenotyping data, when combined with ultrastructural analyses, provide strong evidence that drusen cores are derived from choroidal dendritic cells. The identification of dendritic cell-derived cores in smaller drusen and putative drusen precursors, when combined with our studies that demonstrate the presence of HLA-DR, immunoglobulin light chains, vitronectin, and terminal complement complexes in all drusen phenotypes (see Table 2), suggest a role for dendritic cells and immune-mediated processes in drusen biogenesis and early AMD.

### Example 4

### Further Characterization of Drusen-Associated Molecules: the Development of Procedures for the Enrichment of Drusen from Human Eyes

Reagents: Polyclonal antisera directed against vitronectin (VN) and laminin (LN) were obtained from Telios (San Diego, Calif.); antibodies to collagen type IV were obtained from Chemicon (Temecula, Calif.). Wheat germ agglutinin (WGA) and Limax flavus agglutinin (LFA) were purchased from Vector (Burlingame, CA) and EY Laboratories (San Mateo, Calif.), respectively. Reagents employed in embedding tissues for immunofluorescence were obtained from Bethesda Research Laboratories (Bethesda, Md.) and Sigma Chemical (St. Louis, Mo.). Materials employed in the preparation of tissue for transmission electron microscopy were obtained from Fluka Chemical (Milwaukee, Wis.). Sudan black B was purchased from Poly Scientific (Bay Shore, N.Y.). Reagents used for hematoxylin and eosin staining were purchased from Richard-Allan Medical (Richland, Mich.). Round-tipped surgical blades (Beaver Mini Blade ES, #69) were obtained from Becton Dickinson (Franklin Lakes, N.J.).

Human Donor Eyes: Human tissues were obtained from MidAmerica Transplant Services (St. Louis, Mo.) and the Iowa Lions Eye Bank (Iowa City, Iowa) within 5 hours of death. Following removal of the corneas, donor eyes were cut into quadrants. An inferior saggittal wedge from the ciliary body to the macula was removed from each eye and fixed in either 4% paraformaldehyde or one-half strength Karnovsky's fixative (1/2K) for 2 hours, to assess the presence and morphology of drusen in these tissues. The neural retina was removed from each eye, and individual quadrants were pinned to wax-coated Petri dishes, scleral side down.

Microdissection: Attempts were made to microdissect large drusen using number five forceps or narrow gauge (26G) syringe needles. Drusen were gently separated from the choroid and were washed with lOmM phosphate buffered saline (PBS; pH 7.4) and placed into a Petri dish for photomicrography or into an Eppendorf tube for transmission electron microscopical or biochemical analyses.

Scraped Drusen Preparations: In other experiments, the RPE aspect of the pinned quadrants was gently scraped with a Beaver #69, round-tipped blade to debride Bruch's membrane in areas with large numbers of drusen. Care was taken not to slice through the elastic lamina, by holding the blade at a slight angle and scraping perpendicular to the axis of the blade. Both RPE and drusen were harvested from these regions. Other eyes without macroscopically-visible drusen were also scraped. These preparations, which contain RPE but no drusen, served as controls. The debrided material was collected on the surface of the blade, and was then rinsed off with PBS containing protease inhibitors. The RPE/drusen preparations were spun for 3 min in an Eppendorf microfuge prior to fixation or freezing of the pellet in liquid nitrogen for subsequent biochemical analyses.

For some experiments, enriched drusen preparations were incubated in ice cold distilled water in order to lyse RPE cells. These preparations were then either frozen for electrophoresis or were fixed and processed for immunohistochemistry, as above. In other experiments, the RPE was removed with a stream of buffer (using a 30 gauge needle mounted to a 10 cc syringe) and the Beaver #69 blade was used to debride Bruch's membrane of the remaining drusen.

Tissue Processing In order to determine the efficacy of the scraping technique in removing RPE/drusen from Bruch's membrane, portions of the scraped material and the remaining Bruch's membrane/choroid were fixed in 4% paraformaldehyde and prepared as described above. Cryostat sections of the enriched material and the remaining choroid were collected and employed in histochemical analyses.

Sections of drusen-enriched pellets were stained with 1% Sudan black B, WGA, LFA, and antibodies to vitronectin (VN), laminin (LN), complement C5, and collagen type IV. Lectin and antibody staining was performed as described in above.

Portions of enriched drusen/RPE specimens and post-scraped choroid (without drusen or RPE) from the same eyes, were also preserved in 1/2K fixative and prepared for transmission electron microscopy as described above. Thin sections were taken from blocks of enriched drusen to examine the ultrastructure of these preparations, and sections from post-scraped choroids were prepared to examine the integrity of the Bruch's membrane/choroid complex.

One enriched drusen preparation was fixed in 1/2K as above, rinsed in cacodylate, and dried down on a polylysine coated surface for subsequent examination by scanning electron microscopy. The tissue was dehydrated by critical-point drying, and was sputter coated, as described previously.

Laser Capture Microdissection (LCM): As an additional method for the isolation of drusen and other ocular age-related deposits, we have employed laser capture microdissection (LCM) on frozen sections derived from human donor tissues. This technique allows for the precise identification and isolation of drusen from routinely prepared tissue sections. CapSure™ transfer film is placed on the tissue section surface, the structures of interest are identified, and a low power infrared laser is used to bond the structure of interest onto the film, while the remainder of the tissue section remains adhered to the slide. The instrument delivers precise laser pulses to cells or tissues of interest, trapping them in a polymer film and separating them from the remainder of the tissue components. Laser spot sizes of 7.5, 15, or 30 μm may be selected.

Protein Separation and Analysis: Preparation of enriched drusen proteins for electrophoretic separations involved sonicating total RPE/choroid tissues or enriched RPE/drusen pellets briefly on ice, followed by boiling of samples in sample buffer, as described below. Following removal of corneas, donor eyeswere cut into quadrants. Neural retinae were removed in order to reveal more precisely the extent of RPE pathology, including geographic atrophy, choroidal neovascularization, pigment clumping, and/or drusen. Tissues with advanced degeneration of the RPE were excluded from this study. The presence or absence, and extent, of drusen was determined initially under a dissecting microscope. Eyes with large numbers of drusen or no visible drusen (“controls”) were collected separately. In some cases, different regions of the RPE/choroid complex possessing or lacking drusen were collected from the same eye, to control for donor-to-donor variation in protein levels and mobility. In addition, inferior sagittal wedges from the ciliary body to the macula were removed from each eye, bissected meridionally, and fixed in either 4% paraformaldehyde or 1/2K (see Tissue Processing: Electron Microscopy) for two hours. These tissues were used to determine the extent and phenotype(s) of drusen, by routine histological techniques. For some experiments, the entire RPE/choroid was used. Following assessment of drusen status (outlined above), whole RPE/choroids were peeled from the sclera and immediately frozen in liquid nitrogen.

When needed, these tissues were thawed, sonicated in a minimal volume of isotonic buffer (PBS, pH 7.4) containing protease inhibitors (see Appendix) for 20 bursts, and then centrifuged (11,000×g, 5 min.). Protein concentrations of the supematant fractions were determined using the Micro BCA method (Pierce, Rockford, Ill.). Equivalent amounts of protein from donors with or without drusen (10-50 μg of total protein per sample per lane) were separated by SDS-PAGE as described previously (Laemmli, 1970). For most experiments, extracts from at least four drusen-containing eyes and four age-matched donors without drusen were run simultaneously. For some experiments, samples from young donors without drusen were also included in order to control for age-related changes.

Enriched drusen preparations were compared to whole RPE/choroid preparations on silver stained gels and Western blots. In one experiment, the high molecular weight aggregates at the interface of the stacking gel-separating gel, characteristic of drusen-containing preparations, were excised and analyzed by matrix assisted laser desorption ionization mass spectrometry (MALDI-MS).

Amino acid sequencing was performed at the W. M. Keck Foundation's Biotechnology Resource Foundation (New Haven, CT) as described (Stone, et al., 1990). In some cases, in-gel trypsin digestion of Coomassie-stained gel bands was performed (Stone, et al., 1990), and peptides were identified and matched to their respective proteins based on their molecular mass using matrix assisted laser desorption ionization mass spectrometry (MALDI-MS), as described (Williams et al., 1996). The European Molecular Biology Laboratory and OWL databases were then searched for masses of tryptic peptides of known, as well as conceptually-translated, proteins (Lamand and Mann, 1997). Only cases in which at least five peptides and up to 20% of the protein mass were matched to predicted tryptic fragments were the matches considered significant.

MS/MS of Enriched Drusen: As an additional approach to identify the molecular constituents of drusen and enriched RPE/drusen preparations were collected and digested with trypsin, followed by identification of resultant peptides by mass spectrometry (LC/MS/MS). In other studies, these preparations were separated by two-dimensional SDS-PAGE, individual spots were collected, and analyzed, as above, employing MS/MS.

Results

RPE/Choroid Biochemistry: Prior to the development of enrichment techniques for examining drusen constituents biochemically, proteins from the RPE/choroid complex from donors assessed to have drusen by gross and histological examination were separated and compared to those of donors without drusen. As a function of drusen status, variations in the pattern of proteins were observed with silver staining. RPE/choroid extracts from donors with drusen typically possess a doublet of approximately 35/36 kDa, whereas homogenates from age-matched controls exhibit only a single band at this molecular weight. Of donors with drusen, 75% were found to possess the 35/36 kDa doublet, whereas none of the donors without drusen exhibited this alteration. A second pattern variation coinciding with the presence of drusen is a 120 kDa band which is absent in drusen-containing tissues, but is always present in age-matched controls. These bands were excised from preparative gradient gels, and their constituent proteins were identified by MALDI-MS. These analyses identified interphotoreceptor retinoid binding protein as being present in the 120 kDa band associated with donors without drusen. The corresponding region of the gel from drusen-containing donors contained ceruloplasmin, which was not identified in the control donor band, but did not contain IRBP. Cellular retinaldehyde binding protein (CRALBP) and annexin II were found in samples derived from donors with and without drusen, whereas the 26S protease regulatory subunit (involved in ubiquitin-mediated proteolysis) was found only in this band from donors with drusen.

Isolated Solitary Drusen:

Morphology: Large, individual drusen, relatively free of contaminants, were isolated using the techniques described. Isolated drusen were examined using bright field micrography. They were typically spherical or hemispherical, contained vesicular profiles, and were often associated with a few RPE cells or pigment. Ultrastructurally, this material was comprised of membranous debris and other structural elements characteristic of drusen in situ and fragmented RPE cells.

SDS-PAGE: Individual drusen were dissociated in sample buffer and separated by SDS-PAGE, followed by silver staining or Western blotting. Typically, we were only able to collect 5-20 drusen per eye using this approach. Although too little protein was obtained from each isolated drusen sample to run preparative gels for amino acid sequencing, sufficient material was present for analysis by silver staining and lectin labeling of Western blots. Silver stained drusen preparations typically yielded 6-7 discrete bands ranging in molecular weight from 20 to 65 kDa; these preparations invariably contained a prominent band with a molecular weight of approximately 35 kDa. Lectin labeling of Western blots indicated that isolated drusen contained one major WGA-binding band of approximately 65 kDa, as well as India ink-binding bands of 78 and 62 kDa. Interestingly, vitronectin migrated at 65 kDa under reducing conditions. The 65 kDa WGA-binding band migrated at the same apparent molecular weight as serum albumin. However, the drusen-associated band was bound by silver stain, in contrast to albumin, which was visualized as an unstained band against the background.

Enriched Drusen Preparations:

Histology and Histochemistry: Drusen in situ are typically eosinophilic when stained with hematoxylin/eosin. Small, hard (“hyaline”) drusen stain more intensely and uniformly than large, soft drusen, which tend to be more heterogenous. Drusen in enriched preparations were stained similarly. In eyes in which differences in staining between hard and soft drusen was apparent in situ, this same pattern was also noted in preparations of enriched drusen collected from the same eye. Spherical hard drusen could be discriminated from large, amorphous soft drusen in these preparations. Layers of RPE cells were also readily apparent in these preparations.

The RPE/drusen-debrided choroid, enriched drusen preparations, and intact, control regions from the same eye were examined using immunohistochemistry and lectin histochemistry. The intact basal lamina of the choriocapillaris was observed in the debrided choroid, as was the autofluorescent elastic lamina of Bruch's membrane, providing evidence that Bruch's membrane was not breached during the enrichment procedure.

Antisera directed against VN and C5 and drusen-binding lectins were used as markers to follow drusen through the enrichment process. In the intact RPE/choroid complex, these markers labeled drusen intensely. The globular drusen within the enriched drusen preparations exhibited intense labeling with these probes, indicating that drusen retained VN, Cs, and drusen-associated glyconjugate molecules after enrichment. Similarly, drusen within enriched pellets bound Sudan black B and oil red O in the same manner as was seen in situ.

Exposure of RPE/drusen preparations to water reduced the amount of the RPE cell material in these preparations; only the highly insoluble melanosomes/residual bodies remained in the drusen rich pellet. Drusen remained highly immunoreactive to C5 antibodies after this treatment.

Electron Microscopv: Ultrastructural observations of enriched drusen preparations demonstrated that they contain RPE cells, the RPE basal lamina, free melanosomes from the RPE, and drusen that were morphologically identical to those observed in situ in the same eye. No contamination of the pellets with choroidal material was observed. Basal laminar deposits, that typically lie between the RPE and its basal lamina, were also present in these preparations. The drusen-debrided choroids possessed an intact Bruch's membrane. In eyes without drusen, the RPE monolayer was completely removed and much of the RPE basal lamina typically remained adherent to Bruch's membrane. The elastic lamina and inner collagenous zone of Bruch's membrane were intact and undamaged.

By scanning electron microscopy, enriched drusen preparations were visualized as highly heterogeneous mounds of vesicular profiles resembling drusen; RPE cell debris and melanosomes were also apparent.

SDS-PAGE: In preliminary attempts to determine whether enriched RPE/drusen preparations were useful for analyzing drusen constituents, protein profiles of enriched RPE/drusen preparations were compared to total RPE/choroid protein profiles following SDS-PAGE. These experiments revealed a significant reduction in the total number of bands in the enriched preparations. Particularly notable was a reduction in major choroidal constituents such as serum albumin. Enriched drusen preparations possessed immunoreactive vitronectin and apolipoprotein E at the appropriate molecular weights, confirming that known drusen-associated molecules segregated with the drusen-enriched pellet. Western blots of scraped RPE/drusen exhibited bands of relatively high molecular weight, ranging from 22-150 kDa.

As described above, treatment of enriched drusen preparations with water did not result in a significant loss of labeling of drusen-associated molecules. Water-treated RPE/drusen preparations were compared to whole RPE/choroid and non-water-treated RPE/drusen from the same eye. A further reduction in the total number of bands was observed secondary to lysis in the hypotonic water solution.

As part of an initial study to characterize differences between enriched RPE and enriched RPE/drusen preparations, proteins were separated by SDS-PAGE. In previous experiments, we found that drusen-containing preparations contained significantly more high molecular weight protein at the gel interface than did non-drusen preparations. For this reason, stacking gel interfaces were excised and protein constituents of samples with and without drusen were identified by MALDI-MS. The matching putative proteins included myosin, desmoplakin I/II for the RPE preparation and Myosin, beta-spectrin, alpha-spectrin, and N-acetylglucosamine (GlcNAc) transferase for the RPE/drusen preparations.

Laser Capture Microdissection (LCM): We have tested this system for its ability to collect drusen from a complex tissue section, and have found that the Pix Cell™ LCM system can efficiently and rapidly isolate drusen for further analysis.

MS/MS of Enriched Drusen: A set of molecular candidates for drusen-associated molecules/molecules increased in the RPE-Bruch's membrane in association with drusen have been identified using MS/MS. Differentially-expressed proteins included an upregulation of a neutral pI, ˜30 kDa and a basic, 20 kDa spot in the drusen-containing sample. A number of spots additionally appeared to be downregulated in the drusen-containing sample, ranging from basic to acidic and from ˜15 to 8OkDa. To date, these studies have conclusively identified tissue inhibitor of metalloproteases-3 (TIMP3) and vitronectin in the drusen-enriched sample(s).

Collectively, these data demonstrate that a combination of novel drusen isolation techniques and mass spectrometry are a useful tool for the confirmation of histochemically-identified, and for the identification of previously uncharacterized, DRAMs.

### Example 5

### Characterization of Drusen-Associated Molecules (DRAMs)

Tissues: Eyes from the human donor repository and CDD, ranging in age between 45 and 101 years, were processed within four hours of death. Many of these donors had a documented clinical diagnosis of AMD (including donors with geographic atrophy, choroidal neovascularization, and disciform scars in at least one eye) and one donor was diagnosed with cuticular drusen. Human liver was obtained within 2 hours of biopsy. RPE cells were isolated with 2% dispase within 5 hours of death and were grown in Coon's F-12 media with 10% fetal bovine serum.

Immunohistochemistry: Tissues were fixed and prepared as described in other Examples. Slides were blocked for 15 min. in 0.01M sodium phosphate (pH 7.4) containing 0.85% NaCl, 1 mM calcium chloride, ImM magnesium chloride (PBS/M/C), and 1 mg/ml globulin-free bovine serum albumin (PBS/M/C/BSA). Sections were then rinsed for 10 min. in PBS/M/C, incubated in primary antibody diluted in PBS/M/C/BSA, for one hr., at room temperature. In some cases, sections were pretreated, prior to blocking, with 0.5% trypsin (Sigma, St. Louis, Mo.) for 10 min. as specified by the supplier. Following exposure to primary antibody, sections were rinsed (2×10 min.) in PBS/M/C, incubated in the appropriate fluorescein-conjugated secondary antibody (often adsorbed against human serum) diluted in PBSM/CI/BSA (30 min., room temperature), rinsed (2×10 min.) in PBSI/MC, and mounted in Immumount (Shandon, Pittsburgh, Pa.). Adjacent sections were reacted with secondary antibody alone, as negative controls. Some sections were pre-treated for 10 min with 0.5% trypsin (Sigma; St. Louis, Mo.), or 0.2-0.02 U/ml chondroitinase ABC (Seikagaku; Rockville, Md.), for use in conjunction with antibodies for collagen type IV or various chondroitin sulfate proteoglycans, respectively. Drusen-containing tissues from a minimum of five donor eyes were examined for each antibody.

For negative controls, sections were exposed to PBS/M/C/BSA containing: a) no primary antibody; b) 1% (vol/vol) normal serum; and/or c) antibodies to irrelevant proteins. In some cases, an additional control included adsorption of primary antibody to purified antigen. Positive controls included reaction of antibodies with the extracellular matrices of sclera, choroid, and vitreous; retinal and choroidal basal laminae; retinal interphotoreceptor matrix; and liver. In order to determine the “specificity” of serum protein accumulation in drusen, drusen-containing sections were reacted with antibodies to human albumin (Cappel; Malvern, Pa.) and haptoglobin (Dako; Carpenteria, Calif.).

Results

Reactivities of antibodies with drusen are listed in Table 2 below. In general, all positive antibodies bound to all drusen phenotypes. Controls confirm all antibody reactivities to be specific. In addition, the majority of the antibodies utilized bound to the expected regions of sclera, choroid, RPE, retina, vitreous, and/or other “control” tissues.

TABLE 2
Drusen associated molecules (DRAMs)

| ANTIGEN | SOURCE | DRUSEN |
|---|---|---|
| α1 antichymotrypsin | Dako | + |
| α1 antitrypsin | Dako | −/+ |
| α2 macroglobin | Biodesign | − |
| aFGF | | − |
| AKS | | − |
| Albumin | Cappel | − |
| Actin Bundles | | rare punctate label in ch; dr − (n = 3) |
| Amyloid A | Dako | + |
| Amyloid β | Dako | − to +/− |
| Amyloid P | Dako | + |
| Amyloid Prec Prot | B-M | − |
| Antithrombin III | Calb | +/− |
| Apo A1 | Calb | − |
| Apo E | Calb | + |
| ASPG-1 | | − |
| Atrial Natriuretic Factor | Chemicon | − |
| β2 microglobin | B-M | +/− (mostly −) |
| bFGF | | − |
| Basement Membrane | Chemicon | − |
| Bovine nas. cart. p. | ICN | − |

CD Antigens

| | | |
|---|---|---|
| CD1a | Dako | + |
| CD3 | Pharm | −/+ |
| | Dako | − |
| CD4 | Pharm | +/− (cores) |
| CD8 | Pharm | − |
| CD11a | Hogg | + (cores and cell bodies) |
| CD11c | Hogg | + (cores) |
| CD14 | Dako | + (cores) |
| CD15 | Chemicon | − |
| CD21 | | −/+, RPE basal |
| CD31 | Dako | +/− (cores) |
| CD35 | | −/+ RPE |
| CD40 | | +/− (cores) |
| CD44 | Various | − |
| CD45 | Dako | cells +/− to + (unf only); cores seen prev. |
| | | that coloc w/PNA |
| | Chemicon | ch cells +; some cores + (unfixed only) |
| CD55 | | −, rims/basal RPE + |
| CD59 | | −, rims/basal RPE + |
| CD64 | Dako | +/− (cores) |
| CD68 | Dako | + (cores) var; no rxn on fixed; unf rare |
| | | cores + in 3 of 4 donors, ch cells + |
| | | (among melanocytes) |
| CD83 | | + (cores) |
| CD86 | Dako | + |
| C-Reactive Protein | Dako | − to +/− (VS. DHA) |
| Calcitonin | Dako | − |
| Carbonic Anhydrase | | − |
| Carc Assoc Ag | | − |
| cfms/CSF-1 receptor | | − |
| Chondroitin sulfate | | − |
| Chondroitih 0 sulfate | | − |
| Chondroitin 4 sulfate | | + |
| Chondroitin 6 sulfate | | + |
| Chondroitin sulfate PG | Chemicon | − |
| Collagen I | Southern Biotech | − |
| Collagen II | SB | − |
| Collagen III | SB | − |
| Collagen IV | SB, Chemicon | − |
| Collagen V | SB | − |
| Collagen VI (goat) | | almost all −; ch fibrils in one donor (5 |
| | | donors) |
| Collagen VI (ms IgG) | | no rxn (5 donors) |
| Collagen VI (rb) | | Telios choroid +; occasional labeling of |
| | | RPE BL! (5 donors) |
| Collagen VII | | − |
| Collagen IX | | − |
| Collagenases | | |

Complement

| | | |
|---|---|---|
| C1q | Calb | −/+, positive RPE |
| C1q inhibitor | | −/+ |
| C2 | | − to + |
| C4 | | − to + |
| C3a | | −/+ |
| C3c | | − to + |
| C3d | | + drusen |
| C5 | | + |
| C6 | | + |
| C7 | | + |
| C8 | | + |
| C9 | | + |
| C5-C9 complex | Calb, Quidel, Dako | +/− |
| COS | | − |
| CRALBP | | − |
| Cystatin C | | − |
| Decorin | Chemicon | − |
| Elastin | Sigma | − |
| Entactin | | − |
| Factor D | | + |
| Factor H | | + |
| Factor I | | −/+ |
| Factor B | | − to + (cores), CH+ |
| Ba | | −, CH+ |
| Factor X | Dako | + |
| Fibrillin-1 | | −, Bruch's + |
| Fibrillin-2 | | −, Bruch's +, ch fibrils ++ |
| Fibrin | | − |
| Fibrinogen | Dako | − to +/− |
| Fibronectin | | − |
| Fibulin 3 | Timpl | −/? (+ in BLD) |
| Fibulin 4 | Timpl | −/? (+ in BLD) |
| FnR | | − |
| α Fodrin | | − |
| β Fodrin | | −/+ |
| Gangliosides | Dev Hyb | − |
| Gelsolin | | − |
| GFAP | | − |
| Glucose Transporters 1, 3, 4 | | − |
| Glycolipid | Dev Hyb | − |
| Glycophorin A, C | | − |
| Ham 56 | Don | some bvs +, ch cells +/−, dr − |
| Haptoglobin | Dako | +/− (variable) |
| Heckenlively serum Ag | | +/− |
| Heparan sulfate | (MAB) | +/− |
| | (MAC) | |
| | Kimata | |
| Hermes | | − |
| HLA ABC | | −/? |
| HLA DR | Dako, Mult clones | + (88% +) |
| HNK-1 | | − |
| Heat Shock Prot 27 | | + |
| Heat Shock Prot 60 | | − |
| Heat Shock Prot 70 | | − |
| HSPG | | − |
| Human IgA | | − |
| Human IgG | | +/− |
| Hyaluronic Acid (NDOG) | | − (no rxn; 3 donors) |
| IgGAM | | −/+ |
| IgG | | −/+ |
| IgM | | − |
| Ig Kappa chain | | − to +/− |
| Ig Lambda chain | Dako | +/− to + (var; all dr − in fixed; =/− in |
| | | unfixed; chroid always + in fixed or unf; |
| | | IPM +) |
| Integrin α2 | | − |
| Integrin α3 | | − |
| Integrin α4 | | − |
| Integrin α5 | | − |
| Integrin α6 | | − |
| Integrin β1 | | − |
| Integrin β2 | | − (basal RPE +) |
| Integrin β4 | | − |
| Intermediate Filaments | | − (all − except weak label of GCL, n = 3) |
| Interphotoreceptor Matrix | | − |
| IRBP | | − |
| Keratan sulfate | | − |
| Keratin | | − |
| Laminin | | − (+ in BLD) |
| LAMP-1 | Dev Hyb | − |
| LAMP-2 | Dev Hyb | − (no rxn, 5 donors) |
| Link Protein | Dev Hyb | − |
| Lipoprotein β | | − to +/− |
| Mannose binding protein | | + |
| Mannose receptor | | − |
| MAGP-1 | Elastin Pdts | −, Bruch's + |
| MAGP-2 | Elastin Pdts | −, Bruch's + |
| Melanoma Assoc Ag | | − |
| Milk mucin core Ag | | − |
| MMP1 | | R/Ch −; ILM - to +/− (5 donors) |
| MMP2 | | R/Ch − (5 donors) |
| MMP3 | | R/Ch − (5 donors) |
| MT-1-MMPs (MMP14) | | −, rare cores + ch − to +; occ dr +, most −; |
| | | ELM (?) + |
| Mitochindrial Ag | | − ret CIS +/−; dr, ch −, n = 5 |
| N.S. Enolase | | − |
| Nerve Growth Factor | | − |
| NGFR | | − |
| Neurofibrillary tangles | | − |
| PG40 (Decorin) | | − |
| Phospholipase A2 | | − |
| Plasminogen# | Dako | + (rare dr vesicles +; choroid +/− to +) |
| Plasminogen Act. Inhib. −1 | | − |
| Platelet Derived GF | | − |
| Prealbumin# | B-M | − to + |
| Proteasome 26S s7 | ((MAINE)) | −/+ to +; note that smaller drusen may be |
| | | + whereas large softies are more +/− |
| Protein A | | + |
| Prothrombin# | | +/− (vesicles, not cores) |
| Prothrombin (repeat) | | rare dr +/−; choroid +/− to + |
| S-100 (Bovine) | | −/? |
| Sialo Cell Surface Ag | | − (no rxn) |
| Smoothelin | Chemi | −, SMCs, RPE + |
| SP40, 40 | | + |
| Tau | | − |
| Tenascin | | − sclera, vessels +; IPM + |
| TGFβ | | − |
| Thrombin | Sera | +/−; bvs +/−; dr −; IPM + |
| Thrombospondin | (Gib/AMAC) | − to +/− |
| Thrombosp | AMAC | − (all 6 donors) |
| Thrombosp | Chemi | − (all 6 donors) |
| Thrombosp (IgM) | | ch − except in one spot; dr − to +/−; |
| | | GCL + (3 donors) |
| TIMP1 | | − |
| TIMP2 | | − |
| TIMP3 | Chemicon | + (Drusen +; RIS +?; GCL +) |
| TIMP4 | Chemicon | +/− (Dr cores +, coloc w/PNA; rest −; |
| | | RIS?, GCL+) |
| Tubulin | | − |
| Ubiquitin | StressGen | − to + (v rare cores) |
| UPAR | Anderson | − |
| Vimentin | | − |
| Vitronectin | Various | + |
| VnR | | − |
| von W Factor | | − (most dr −; rare dr +; bvs +/++) |

Key: ++ = intense, invariant labeling; + = strong labeling in most donors; +/− = weak labeling; − = no labeling detected; (var.) = donor to donor or drusen to drusen variation; vesicles = labeling of spherical profiles within drusen; B-M = Boehringer-Mannheim; Calb = Calbiochem; Gib = Gibco/BRL; Pharm = Pharmingen; Sera = Sera Labs; Tel = Telios;

### Example 6

### Drusen Associated with Aging and Age-Related Macular Degeneration Contain Proteins Common to Extracellular Deposits Associated with Other Diseases

Recent studies in this laboratory revealed that vitronectin is a major component of drusen. Because vitronectin is also a constituent of abnormal deposits associated with a variety of diseases, drusen from human donor eyes were examined for compositional similarities with other extracellular disease deposits. The sixty-three human donor eyes employed in this study were obtained from The Human Donor Repository and the CDD. All eyes were collected and processed within four hours of death; donor ages ranged from 45 to 96 years. Drusen were categorized as hard or soft. Tissues from a minimum of five donors were assayed with each antibody employed, at least two of whom had clinically-documented AMD, and each drusen phenotype was examined in at least two donors. Institutional Review Board committee approval for the use of human donor tissues was obtained from the Human Subjects Committee at The University of Iowa.

Thirty-four antibodies to twenty-nine different proteins or protein complexes were tested for immunoreactivity with hard and soft drusen phenotypes. These analyses provide a partial profile of the molecular composition of drusen (see Table 3 below). Serum amyloid P component, apolipoprotein E, immunoglobulin light chains, Factor X, and complement proteins (C5 and C5b-9 complex) were identified in all drusen phenotypes. No reaction of antibodies to the primary amyloid proteins keratin, apolipoprotein A-I, gelsolin, calcitonin, atrial natriuretic factor, tau, or amyloid precursor protein was observed. Antibodies against human serum albumin and haptoglobin bound strongly to the choroidal stroma, but not to hard or soft drusen. Immunoreactivity of some drusen-associated proteins was frequently observed in distinct, heterogeneous patterns. For example, drusen binding by prothrombin and amyloid A antibodies, was often localized to spherical profiles within drusen. Drusen were occasionally labeled by anti-fibrinogen antibodies; this binding was generally confined to peripheral regions and/or concentric bands within drusen.

The compositional similarity between drusen and other disease deposits may be significant in view of the correlation between AMD and various systemic disorders. These data suggest that similar pathways may be involved in the etiologies of AMD and other systemic disorders.

TABLE 3
Immunoreactivity of Drusen

| Antigen | Supplier | Conc. | No. | Drusen |
|---|---|---|---|---|
| Albumin | Accurate | 1:50 | 5 | − |
| Amyloid A | Dako | 1:50 | 8 | +/−; vesicles |
| Amyloid β | Dako | 1:10 | 7 | − |
| Amyloid Precursor | Boehringer | 1:20 | 5 | − |
| Protein | Mannheim | | | |
| Amyloid P | Dako | 1:50 | 6 | ++ |
| component | Calbiochem | 1:50 | 5 | ++ |
| α1-antichymo- | Dako | 1:50 | 6 | +/− (var.) |
| trypsin | Calbiochem | 1:50 | 5 | +/− (var.) |
| α1-anti-trypsin | ICN | 1:50 | 5 | −, rare +/− |
| Apolipoprotein A1 | Calbiochem | 1:50 | 6 | − |
| Apolipoprotein B | Chemicon | 1:20 | 6 | − |
| | Dako | 1:50 | 5 | − to +/− |
| Apolipoprotein E | Calbiochem | 1:50 | 9 | + |
| Atrial natriuretic | Calbiochem | 1:50 | 5 | − |
| factor | | | | |
| C-reactive protein | Dako | 1:50 | 5 | − to +/−, (var.) |
| Calcitonin | Dako | 1:50 | 5 | − |
| Complement C 1q | Calbiochem | 1:50 | 5 | − |
| Complement C3 | Dako | 1:50 | 5 | − to +, (var.) |
| Complement C5 | Dako | 1:50 | 5 | ++ |
| Complement C5b-9 | Dako | 1:50 | 5 | ++ |
| Cystatin C | Accurate | 1:50 | 5 | −, (var.) |
| Factor X | Dako | 1:50 | 9 | + |
| Fibrinogen | Dako | 1:50 | 5 | − to +/−, (var.) |
| Gelsolin | Chemicon | 1:50 | 5 | − |
| HLA-DR | Accurate | 1:25 | 10 | + |
| | Dako | 1:200 | 10 | + |
| Immunoglobulin | Boehringer | 1:50 | 8 | − to +/− |
| kappa | Mannheim | | | |
| Immunoglobulin | Dako | 1:50-1:2000 | 9 | +/− to + |
| lamda | | | | |
| β2 microglobulin | Boehringer | 1:50 | 5 | − to +/− |
| | Mannheim | | | |
| Prothrombin | Dako | 1:50 | 5 | + (vesicles) |
| Tau | Dako | 1:50 | 5 | − |
| Transthyretin | Boehringer | 1:50 | 9 | +/− (var.) |
| | Mannheim | | | |
| Ubiquitin | Chemicon | 1:50 | 5 | − |
| | StressGen | 1:100 | 5 | −, rare +/− |

Key: ++ = intense, invariant labeling; + = strong labeling in most donors; +/− = weak labeling; − = no labeling detected; (var.) = donor to donor or drusen to drusen variation; vesicles = labeling of spherical profiles within drusen

### Example 7

### Local Sources of DRAMs Common to Extracellular Deposits Associated with other Diseases

Studies were conducted to determine whether any of the DRAMs that were identified as being common to extracellular deposits associated with atherosclerosis, elastosis, amyloidosis, or dense deposit disease were produced locally in the eye by RPE, retinal, and/or choroidal cells.

RNA Isolation: Total RNA was isolated from adult human liver, RPE/choroid, retina, and enriched RPE as described by Chirgwin et. al. (1979), except that cesium trifluoroacetate was used instead of cesium chloride in the density gradient ultracentrifugation step. The resulting pellet was stored at −80° C. The quality/integrity of RNA obtained was assessed on both agarose gels and Northern blots. Total protein was determined from identically sized punches of the ocular tissue(s) from which the RNA was collected and employed as an internal reference.

RT-PCR Analyses: Total RNA was extracted from the specified tissues and cDNA was synthesized with reverse transcriptase using oligo(dT)₁₆ as a primer. Reverse transcriptase was omitted from some reactions. cDNA was amplified using molecule-specific primer pairs. PCR amplification products were separated electrophoretically on a 1.8% agarose gel.

Results: Transcripts encoding a number of DRAMs common to extracellular deposits associated with atherosclerosis, elastosis, amyloidosis, or dense deposit disease were found to be synthesized by the retina, retinal pigmented epithelium and/or choroid (see Table 4 below).

TABLE 4
RT-PCR results from retina, RPE/choroid, and liver.

| Gene Name | Ret | R/Ch | RPE | Gen | Liver |
|---|---|---|---|---|---|
| Albumin | + | + | + | − | + |
| Amyloid P | − | − | − | − | + |
| Apo B | + | + | − | − | + |
| Apo E | + | + | + | − | + |
| Complement | + | + | + | − | + |
| 3 | | | | | |
| Complement | + | + | + | − | + |
| 5 | | | | | |
| Complement | + | + | − | − | + |
| 9 | | | | | |
| Factor X | + | + | − | − | + |
| Fibrinogen | − | + | − | − | + |
| Ig kappa | − | + | − | − | + |
| Ig lamda | + | + | − | − | + |
| Prothrombin | − | − | − | − | + |

Ret = retina; R/Ch = RPE/choroid; Gen = amplification of genomic DNA by the primer pair;
* = higher molecular weight genomic band detected with primer pair.

### Example 8

### Characterization of Metalloproteinases/TIMPs in AMID Donors

Extracellular matrix turnover is initiated, at least in part, by the regulated secretion of members of a family of matrix metalloproteinases (MMPs) and their inhibitors, the tissue inhibitors of metalloproteinases (TIMPs). Leukocytes, including dendritic cells and macrophages, are major sources of MMP production. MMP action permits leukocyte immigration into tissues, causes tissue damage, and generates immunogenic fragments of normal proteins that may escalate autoimmune diseases (Opdenakker, 1992). The MMP family of enzymes contributes to both normal and pathological tissue remodeling. Although the link between single MMPs and individual substrates is not as direct as once thought, it is clear that the MMPs are capable of breaking down most ECM components. Most MMPs, with the exception of the 72 kDa gelatinase and the MT-MMPs, are not constituitively expressed in normal tissues. Inflammatory cytokines (IL-1 and TNF) and growth factors (TGFβ) are typically required to initiate transcription. MMPs are expressed as inactive zymogens, which are activated extracellularly by the action of enzymes such as plasmin and other MMPs. Once activated, MMPs are subject to inactivation by TIMPs and by binding to plasma proteins such as a2-macroglobulin. This balance of expression and activation, and the levels of TIMPs, govern the level of destruction mediated by MMPs. Excessive or inappropriate expression of MMPs may contribute to the pathogenesis of many tissue destructive processes, including diseases such as arthritis, multiple sclerosis, atherosclerosis, and COPD.

In order to assess the notion that an imbalance of the metalloproteinase/inhibitor system in AMD may lead to degradation of Bruch's membrane in the macula, RPE/choroidal tissues from 20 donors with and without AMD were examined using zymography. Four proteases with approximate molecular weights of 65 (MMP-2), 95 (MMP-9), 120, and 250 kDa were present in macular and peripheral tissues. No differences in the pattern of MMP bands were detected as a function of age or drusen phenotype. Aprotinin and leupeptin had no effect on proteolytic degradation of gelatin, whereas EDTA (5 mM) completely inhibited enzyme activity in these bands, indicating that all four bands are likely metalloproteinases. These activities were also resistant to boiling, but extremely sensitive to reducing agents.

In order to establish the relationship of lysed bands on zymography with known metalloproteases, antisera to a number of known MMPs, as well as to all known TIMPs, were employed to screen Western blots of RPE/choroid proteins. MMP-1, -2, and -9, but not MMP-3 or -8, were identified in RPE/choroid extracts and did not show changes with respect to drusen status, AMD, and/or age. TIMP-4 antibody bound to a band of 28 kDa in all samples, including a 2-month-old donor. The higher molecular weight bands may be due to smaller MMPs that have polymerized (99) or may represent novel proteases, such as the 300 kDa elastase identified in lung by broncheoalveolar lavage (100). Antibodies directed against TIMP-3 reacted with hard and soft drusen, whereas anti-TIMP-4 antibodies reacted with drusen cores.

The development of a comprehensive picture of MMP involvement will require the use of several methods. Ongoing studies are being focused on further characterization of MMPs 2, 7, 9,-12, and 14, members of the MMP. family that exhibit elastolytic properties, using immunohistochemistry, zymography, ELISA, and QRT-PCR.

### Example 9

### Autoantibodies in the Sera of Donors with AMD and/or Drusen

In order to address the role of autoantibodies in drusen biogenesis and AMD, we performed a series of preliminary experiments using enriched drusen preparations in order to identify anti-drusen/Bruch's membrane/RPE autoantibodies that might be present in the sera of donors with AMD and/or drusen.

Protein extracts from an enriched drusen preparation (DR+) obtained by debridement of Bruch's membrane with a #69 Beaver blade and from a control (DR−) preparation were prepared using PBS with proteinase inhibitor cocktail and mild detergent. Proteins were separated by molecular weight using 10-20% gradient mini SDS gels (Amresco) and transferred to PVDF membranes for Western blot analysis. PVDF strips with human retinal proteins from 50 normal human retinas were also used for detection of any anti-retinal autoantibodies in the donor sera.

Sera from the same eight donors described above were screened. Serum from one AMD donor (#90-98) positively labeled a band in the RPE (both DR+ and DR−) and RPE/choroid preparations of approximately 35 kDa. A second band of approximately 60 kDa was labeled weakly only in the DR+ protein extract. Sera from an AAA donor (#189-97) reacted with a protein(s) of approximately 53 kDa. This band labeled in all three protein extracts. There was one band of approximately 64 kDa that this serum sample labeled only in the DR+ sample.

The presence of serum anti-drusen/RPE autoantibodies in donors with AMD and/or drusen further suggests a possible role for shared immune-mediated processes in these conditions.

### Example 10

### Analyses of Autoantibodies in the Sera of Living AMD Patients

In order to determine whether the sera of AMD patients possesses autoantibodies or alterations in the abundance and/or mobility of serum proteins, plasma was collected from 20 patients with clinically-diagnosed AMD and from 20 unaffected patients to serve as controls.

For some experiments, sera were separated by SDS-PAGE and proteins were visualized with either silver stain or Coomassie blue, or (for preparative purposes) proteins were transferred to PVDF membranes for amino acid sequencing. Abnormalities of serum proteins were detected in a subset of AMD donors. These differences included the presence of “additional” bands in the sera of some AMD patients (molecular weights of ˜25, 29, 30 and 80 kDa) that were not present in control donors. Amino acid sequencing of these molecules revealed N-terminal sequences consistent with haptoglobin (25 kDa) and immunoglobulin kappa (29 kDa), lambda (30 kDa), and gamma (80 kDa) chains.

In a second set of experiments, sera from AMD and control donors was screened for the presence of auto-antibodies. As an extension of experiments in which weak-moderate immunoreactivity of drusen in tissue sections was previously observed, purified vitronectin was electrophoretically separated and blotted onto PDVF. Because vitronectin had been identified as a DRAM (see, Examples 4 and 5), the sera from AMD patients was then evaluated for the presence of anti-vitronectin immunoreactivity. Strong labeling of both the 65 kDa and 75 kDa vitronectin species was identified in these sera, indicating that AMD sera contain autoantibodies directed against at least some DRAMs and/or Bruch's membrane constituents.

As an additional approach toward the identification of AMD autoantibodies and their targets in ocular tissues, RPE-choroidal proteins from one donor with large numbers of drusen and a nine month old donor were separated electrophoretically according to molecular weight and transferred to nitrocellulose. Proteins were then immunolabeled with either sera from 3 AMD donors or polyclonal antiserum directed against vitronectin. The AMD sera reacted with bands of roughly 65, 150 and 200 kDa only in the sample from the donor with numerous drusen. These results are suggestive that age and/or the presence of drusen leads to an increase in AMD autoantigen.

### Example 11

### Additional-Assessment of Additional Serum Markers in Drusen Biogenesis and AMD

Study Design: Visual acuity measurements, stereo macula photos, and peripheral photos will be taken at the beginning of the study and every six months thereafter. Blood and sera will be drawn when subjects enter the study and every 6_{—}12 months thereafter. DNA will be prepared from a portion of each blood sample for future genetic studies. The presence of serum autoantibodies and immune complexes will be determined using standard protocols. In addition, sera will be reacted with tissue sections derived from donors with and without AMD, followed by a secondary antibody that has been adsorbed against human immunoglobulins. Western blots of retina/RPE/choroid from AMD and non-AMD donors will also be incubated with serum samples to identify specific bands against which autoantibodies react.

In addition, levels of the following proteins, additional indicators of autoantibody responses, chronic inflammation and/or acute phase responses, will be assayed by a clinical diagnostic laboratory. These will include Bence Jones protein, serum amyloid A, M components, C-reactive protein, mannan binding protein, serum amyloid A, C3a, C5a, other complement proteins, coagulation proteins, fibrinogen, vitronectin, CD25, interleukin 1, interleukin 6, and apolipoprotein E. Serum protein electrophoresis, lymphocyte transformation, sedimentation rate, and spontaneous, whole blood, white cell count will also be measured.

The presence of antibodies directed against the following proteins (many observed in other age-related conditions and/or MPGN) will also be determined: type IV collagen, glomerular basement membrane, neutrophils, cytoplasm (c-ANCA, p-ANCA), C3 convertase (C3 nephritic factor), alpha-1 anti-trypsin levels (decreased in MPGN), epsilon 4 allele, apolipoprotien E, GFAP, ANA, serum senescent cell antigen, S-100, type 2 plasminogen activator, alpha-1-antichymotrypsin, SP-40,40, endothelial cell, parietal cell, mitochondria, Jo-1, islet cell, inner ear antigen, epidermolysis Bullosa Acquista, endomysial IgA, cancer antigen 15-3, phospholipid, neuronal nucleus, cardiolipin, and ganglioside.

Other markers that could be present in the serum of patients having a drusen associated ocular disorder are listed in the following Table.

TABLE 5
Serological Tests for Immune Mediated Processes, Including
Autoimmune Disease and Chronic Inflammation

Cells:
Whole blood cell count, hemogram plus differential
CBC, hemogram.
Immunoglobulins:
Immunoglobulin A, G, M, D, E quantification
IgG subclass quantification
Kappa/lambda light chains quantification and ratios
Miscellaneous Proteins:
Serum protein electrophoresis
Complement, total classical and alternative
Complement: C3, C4, C5 quantitative
Bence Jones proteins
M component
C reactive protein
Serum amyloid A
Coagulation proteins
Fibrinogen (and/or ESR)
Elastase inhibitors
Elastin and collagen peptide fragments
Serum β-2-microglobulin
Serum carotine
Creatine kinase
Rheumatoid factor
C-reactive protein
Immunocompetent Cells:
Lymphocyte immunophenotyping and absolute CD4 cell count.
Anti-OKT3, IgG antibodies.
CD34 Stem cell count.
CD3 cell count.
CD4 cell count.
Lymphocyte mitogen and antigen profile screen (LPA).
Lymphocyte antibody screen???
NK cells.
T and B-cell markers. (which ones they screen?).
CD4/CD8− absolute count and ratio.
HLA phenotyping, both class I and II. HLAB-27.
Cytokines:
Interleukins
Fibroblast growth factor
Vasoactive intestinal peptide (VIP)
Autoantibodies:
Anti-nuclear antibody (ANA)
Anti-neutrophil cytoplasmic antibody (ANCA)
Double stranded DNA antibody
Anti-ribonuclear protein antibody
Scl-70 antibody
SM antibody
SS-A antibody (anti-RO) and SS-B (anti-LA) antibody
Anti-neuronal nuclear antibodies
Antineuronal nuclear antibody (Purkinje cells).
Jo-1 antibody
Paraneoplasctic antibody A
Anti-cardiolipin antibody
Anti-glomerular basement membrane antibodies
Mitochondrial antibody
Anti-ganglioside assay
Anti-Streptolysin-O screen
Anti-sulfatide antibody
Anti-Thyrocellular antibody
Antibody to inner ear antigen
Bullos pemphigoid antibodies
PM-1 antibody
Adrenal cortical antibody.
Liver-kidney microsomal antibody
Mitochondrial antibody
Parathyroid antibody
Parietal cell antibody
Pemphigus antibodies
Smooth muscle antibodies and striated muscle antibodies.
Islet cell antibodies
Lupus anticoagulant
Anti-Viral and Anti-Bacterial Antibodies:
CMV antibody
Group B strep antigen
Hepatitis B, E, C, A antibodies
Helicobacter Pylori antibodies
Antibodies to CMV, EB virus, Herpes Simplex, Measles,
mycoplasma, Rubella, Varicella-Zoster
Others:
Cancer antigen 125
Cancer antigen 15-3
Carcinoembrionic antigen
Small fiber axonal profile
CNS serology battery
Sensorimotor neuropathy profile

### Example 12

### Differential Gene Expression Analyses in the RPE/Choroid Complex of Donors with AMD: Toward the Development of a Diagnostic “Gene Expression Fingerprint” for Drusen Biogenesis and AMD

One prevailing concept pertaining to the etiology of AMD is that a threshold event occurs at some point during the aging process that distinguishes AMD from normal aging. Provided that AMD is heritable in the majority of affected individuals, then the gene(s) responsible likely initiate this threshold event. Our working hypothesis suggests that cellular dysfunction within the RPE-choroid-retina complex is involved in the earliest stages of AMD, since most of the initial clinical and histopathological signs (e.g. RPE cell death) are associated with the RPE, Bruch's membrane, and the choroid. However, little is known about the patterns of gene expression in normal RPE and choroidal cells and nothing is known about gene expression in RPE, choroidal, or retinal cells from individuals with AMD and/or drusen formation. This is especially surprising in view of the strategic location of the RPE, the fact that its health appears crucial for the maintenance of the retina-choroid interface, and its apparent involvement in AMD. Because of our access to a large repository of carefully documented human donor eyes, we are in a unique position to determine unique patterns of RPE, choroidal, and retinal cell gene expression (AMD and drusen “gene expression fingerprints”) in defined AMD phenotypes that are distinct from those of age-matched and younger donors without AMD.

Differential gene expression of RPE/choroid complexes derived from four paired donors of selected AMD phenotypes and age-matched controls has been analyzed using gene array analysis. The arrays utilized in this study contained 18,380 non-redundant cDNAs derived from the I.M.A.G.E. consortium. Each cDNA clone was robotically spotted, in duplicate, onto a nylon membrane in a precise pattern, allowing easy identification. These analyses are typically performed using first strand cDNA which has been radiolabeled during reverse transcription of the probe mRNA. However, due to the small amounts of mRNA that can be isolated from the RPE layer of individual human donor eyes, we have modified this standard protocol. The cDNAs were radiolabeled with ³³P in a random-primed reaction, purified, and hybridized to the gene arrays. The arrays were phosphoimaged, the signals were normalized, and the data analyzed using the Genome Discovery Software package (Genome Systems).

Analysis of the data reveals distinct patterns of clones that are significantly up- and/or down-regulated in the RPE/choroid of individuals with specific AMD as compared to controls. At this point, these differentially-expressed mRNAs can be grouped into three distinct “pathways”: extracellular matrix, membrane transport, and gene regulation-associated pathways. In addition, a significant number of uncharacterized expressed sequence tags (ESTs) are differentially expressed in the RPE-choroid of donors with specific AMD phenotypes as compared to the RPE from donors without the disease.

It is anticipated that large scale analyses of gene and protein expression profiles (“fingerprints”) in tissues from donors with drusen deposits, as well as those from various “AMD phenotypes”, will provide significant new insight into the molecular pathology of cell dysfunction associated with the development of AMD. These “gene expression fingerprints” will also provide powerful diagnostic capabilities for detecting individuals at risk for developing drusen and/or macular degenerations, including AMD.

### Example 13

### RPE Cell Death as Related to Drusen Biogenesis

RPE Cell Density as a Function of Age: RPE cells are generally considered terminally-differentiated. Thus, there is no mechanism for the replacement of lost cells in vivo. Although the net density of RPE cells appears to decrease in human eyes as a function of age, the rate of loss has not rigorously examined. Based on data from preliminary studies, we propose to determine whether this loss is linear with age, varies in peripheral and macular regions, is greater in eyes from donors with AMD, especially early AMD, and, if so, whether this loss is associated with drusen phenotype.

In order to test the feasibility of determining the relationship between RPE cell density, age and drusen status, we counted RPE nuclei on DAPI-stained sagittal sections (DAPI is a probe that binds specifically to DNA), spanning from the ora serrata to the macula of each quadrant, in a series of 20 CDD donors with and without drusen/AMD. The number of RPE nuclei, basal drusen length and drusen area were determined per mm of Bruch's membrane. Sections were also photographed at 500 nm excitation to assess qualitatively the degree of autoflourescence due to RPE lipofuscin.

These data suggest that both RPE cell density and thickness (volume) is reduced in eyes with numerous drusen/AMD, supporting our hypothesis that RPE may contribute to drusen biogenesis. Four additional phenomena have been observed. First, a significant decrease in RPE density is noted in the peripheral and equatorial retina in some donors between the ages of 10 and 40. Second, although many RPE cells appear to be extruded toward the subRPE space where they contribute to drusen formation, others are extruded into the subretinal space, often as groups of cells, and are “phagocytosed” by the neural retina. Third, focal groups of RPE cells that express HLA-DR were identified. Fourth, morphological images are suggestive that the neural retina underlying drusen, even small drusen, exhibits thinning and local reduction in nuclear number.

RPE Contribution to Drusen Development: The distribution of RPE-associated lipofuscin and/or pigment granules, in addition to nuclei, is easily detected in DAPI-stained sections. Examination of DAPI-stained preparations revealed the presence of autofluorescent lipofuscin and pigment within small drusen, as well as dispersed profiles of DAPI-reactive material, interpreted to be RPE nuclei. When tissues from the same donors are examined ultrastructurally, profiles of lipofuscin and pigment were detected in small drusen. Sometimes these were contained within membrane bound fragments of RPE cells. Nuclei or nuclear material are observed within the drusen less commonly. In a few eyes we have observed the remnants of whole RPE cells in the sub-RPE space. Significantly, drusen-associated dendritic cell processes are often observed in association with these profiles of RPE cells, suggesting that the dendritic cells are initially recruited to these sites of RPE damage.

Focal RPE “Injury” and Drusen Development: We have observed focal groups of RPE cells that express HLA-DR. These cells are often associated with small drusen and exhibit unique apical-basal polarization, and apical displacement of the majority of cellular organelles. Additional studies of these cells indicate that that they react with antibodies directed against CD68, HLA-DR, vitronectin, clusterin, and apolipoprotein E. In diseases such as rheumatoid arthritis and glomerulonephritis, immunoglobulin and complement activation are associated with tissue injury and subsequent cell death.

Mode of RPE Cell Death: We have neverobserved an apoptotic RPE cell in examination of over 35,000 electron micrographs. In addition, the morphological profiles of dead or dying RPE cells that we observe exhibit the hallmarks of necrotic, rather than apoptotic, cell death. Based on the hypothesis that the typical mode of RPE cell death occurs by necrosis, we initiated studies to identify apoptotic RPE cells in sections of 20 human donor eyes of various ages, with and without AMD, from the CDD. All experiments were run in duplicate or triplicate. Although TUNEL positive cells were observed consistently in the RCS rat and human controls, no apoptotic RPE cells were identified in human donor sections, at any age or disease state. These data support the hypothesis that the majority of RPE cell death occurs via cellular necrosis, rather than by apoptosis.

### Example 14

### Drusen Biogenesis in Immunosuppressed Patients with AMD and/or Drusen

Experimental Approach:

Rationale: Based on the hypothesis that drusen formation is dependent on the activity of choroidal dendritic cells, it is likely that drusen formation will be arrested (or that the rate of drusen formation will be reduced significantly) in the absence or down-regulation of these cells. The overall goal of this study is to determine the prevalence, biomicroscopic characteristics and clinical course of preexisting drusen in AMD patients whose immunocompetent cells have been significantly depleted.

Patient Population: Twenty AMD patient volunteers will be identified who have undergone, or will undergo, organ transplants. In some cases, immune compromised patients will be identified for whom ophthalmic medical histories, fundus photographs, and angiograms were collected prior to the immunosuppression. In other patients who will undergo transplantation, fundus photographs and angiograms will be collected before radiation treatment and/or immunosuppression. A target study population will be patients who are to undergo cardiac transplant, as these patients with be likely to have a high incidence of drusen but will have limited life expectancy. The control population will be age-matched patients with and without AMD.

Study Design: In all participants, visual acuity measurements, fuindus photographs, and fluorescein angiography and a number of parameters will be measured at the time of recruitment every six months and compared to age-matched controls with and without AMD. Blood and sera will be drawn when subjects enter the study and every 6-12 months thereafter. DNA will be-prepared from a portion of each blood sample for future genetic studies.

Fundus photographs will be graded by masked readers utilizing the International Age Related Eye Disease Study grading protocol. Measurements will include: total drusen area; sizes of drusen; number of macular drusen; percent of drusen possessing funduscopic “cores”; rate of appearance of new drusen; and rate of regression of extant drusen.

The fundus photographs from the same patient, taken before and for 3-5 years after transplantation (including the control patients), will be compared for the presence of drusen, changes in the distribution and number of drusen, and other drusen related pathology.

### Example 15

### In Vitro Model for Drusen Biogenesis. RPE-Dendritic Cell Interactions, and Gene Expression

Our hypothesis of drusen biogenesis predicts that the essential elements to drusen formation-dendritic cells and the RPE-may interact across Bruch's membrane in the aging eye, via a set of molecular signal molecules, to result in drusen deposition and growth. It is anticipated that this interaction may be reproduced in vitro, as both cell types are tractable to cell culture methodologies. Due to the fact that there is not currently a suitable animal model for drusen biogenesis or AMD, the prospect of testing potential therapeutics which interfere with this interaction in vitro is particularly attractive. Molecules to be tested for their ability to inhibit RPE-dendritic cell interaction may include antibodies directed against cell surface proteins, cytokines and chemokines; growth factors and growth factor inhibitors; anti-inflammatory agents; and inhibitors of matrix degradation by dendritic cells.

Experimental Approach:

Rationale: Based on the histochemical and ultrastructural observations that indicate a role for dendritic cells (DCs) in the formation of drusen, the interactions between RPE cells and DCs will be evaluated in vitro. These studies will aid our understanding of the putative roles of DCs in drusen formation by examining the effects of these cells on stimulation or depression of the expression of specific genes by the RPE and, subsequently, the effects of DC contact and/or DC-secreted molecules on RPE gene expression. These studies will also likely reveal downstream (i.e., RPE) molecular targets for therapeutic intervention, particularly important in the event that general/systemic inhibition of DC activity is assessed to be too global to be effective in the management or prevention of AMD.

RPE Cell Cultures: Human donor eyes, obtained within 4 hours postmortem, will be employed in these studies. We have found that RPE cells can be successfully cultured up to 12 hours after death, making 4 hours a conservative interval for the isolation of RPE cells. RPE cells and other tissues can be isolated from donors with diagnosed AMD, as well as from age-matched donors without AMD.

RPE cells can be isolated either by debridement of the choroid and collection of RPE cells from different defined regions or surgical removal of the sclera from the choroid and removal of RPE cells by incubation of the eyecup in dispase. Our laboratory has considerable experience in the successful application of both protocols for the isolation of RPE. For debridement of the choroid, the eye will be quartered and photographed, the neural retina will be removed, and the RPE surface will be scraped gently with a Beaver #69, round-tipped blade to debride Bruch's membrane in areas with large numbers of drusen. Care will be taken not to slice through the elastic lamina, by holding the blade at a slight angle and scraping perpendicular to the axis of the blade. The debrided material will be collected on the surface of the blade, and then rinsed off the blade with Coon's F-12 culture medium supplemented with either fetal calf serum (10%) or human serum. For isolation of RPE cells from whole eyecups following dissection of the sclera, the protocol described by Pfeffer et al. can be employed, except that Coon's F-12 medium is used for all experiments.

Dendritic Cell Cultures: DCs will be isolated using standard techniques. Briefly, CD14 positive cells from the buffy coat fraction of peripheral blood will be isolated, either from eye donors or from clinic patient volunteers. In some experiments, stimulation of these cells with the appropriate cytokines (GM-CSF and IL-4) will be performed prior to co-incubation with RPE cells in order to pre-differentiate these cells into an activated dendritic cell phenotype. Unactivated DCs will be employed in other experiments. We will also attempt to design a protocol to isolate DCs directly from the choroid.

RPE and Dendritic Cell Co-Culture: RPE cells derived from at least five AMD and five non-AMD donors will be examined for their ability to recruit, or elicit migration of, activated and unactivated DCs. These experiments will be conducted using modified Ussing chambers. Changes in the expression of various DC markers indicative of activation, including IL-12, MHC class II antigens, CD83, and CD14 will be monitored immunohistochemically and using RT-PCR. These experiments will help test our hypothesis that RPE cells from AMD patients are sublethally “injured” and/or secrete “factors” that result in the recruitment and maturation of DCs, thus initiating an inflammatory response that culminates in drusen biogenesis.

In a second set of experiments, RPE and dendritic cells will be plated on the opposite side of a porous membrane, using cell culture inserts. Coon's F-12 medium will be used in both chambers. RPE cells will be plated at confluency and DCs will be plated at a density of 600 cells/mm², which reflects their in vivo distribution. Pore sizes will be employed that will permit cell processes to penetrate (e.g. 1 μm) or that will permit only soluble molecules to traverse (e.g. 0.45 μm). RPE cells will be co-cultured with non-immune cells (e.g. fibroblasts; “sham-stimulated” or with no cells as a control for “normal” RPE gene expression.

Following various periods of time in co-culture, RPE cells and DCs will be collected by either scraping the membrane surfaces or by trypsinization. Cells will be pelleted and RNA will be isolated as described by Chirgwin. Culture supernatants will be collected and frozen for future analyses of proteins, including various growth factors and cytokines. RNA will be isolated from 1) AMD-derived, DC-stimulated RPE; 2) non-AMD-derived, DC-stimulated RPE; 3) AMD-derived, unstimulated/sham-stimulated RPE; 4) non-AMD-derived, unstimulated/sham-stimulated RPE; 5) DCs co-cultured with AMD-derived RPE; and 6) DCs co-cultured with non-AMD-derived RPE. RNA will be reverse-transcribed, and 33P-labeled cDNAs will be employed to probe gene arrays. The data collected from the gene array analyses will be analyzed as described in Objective 1, in order to identify pathways that are up- and/or down-regulated in DCs co-cultured with RPE derived from AMD and non-AMD donors.

### Example 16

### Further Evidence Linking Drusen Biogenesis, Immune-Mediated Processes and Macular Degeneration

T cell proliferation assays, determination of serum immune complexes and Th1/Th2 cytokine profiles, identification of RPE/choroid autoantigens, characterization of chemokine and chemokine receptor expression, and assessment of choroidal CD4/CD8 T cell distribution in a group of AMD and non-AMD patients have been conducted. In addition, serum levels of various markers of inflammation and excessive complement activity are being assessed in individuals with and without AMD. Results from these analyses indicate that RPE antigens are capable of stimulating T cell proliferation in cells derived from at least some individuals with AMD.

Immune Mediated Processes: Experiments to pursue the identification of autoimmune antibodies and immune complexes in the sera and ocular tissues of patients/donors with AMD have also been conducted. Plasma from over 80 patients with or without clinically diagnosed AMD has been assayed to determine whether sera of AMD patients possess autoantibodies directed against specific RPE, retinal, and/or choroidal antigens. Seven distinct protein bands have been identified that react with sera derived from some AMD, but not any control, donors and patients. The antigens recognized by these antibodies include serotransferrin, keratins 9 and 10, apolipoprotein AI, and complement component 1. Autoantibodies directed against serotransferrin and keratin were also detected in the serum derived from a single patient with Malattia leventinese, another hereditary form of macular degeneration.

The presence of immunogobulin (Ig) in enriched drusen preparations has been confirmed by ELISA and Western blot analysis and have sought to develop protocols for characterizing antigen specificity and isotype. To date, approximately 30 μg of drusen-associated Ig has been isolated per eye. This amount has been sufficient to confirm Ig purity and specificity on SDS-PAGE, Western blot and ELISA. Analysis has also been conducted to identify the primary immunoglobulin isotype in an enriched drusen preparation derived from a donor with substantial numbers of drusen and a diagnosis of AMD. Analysis of immunoglobulin associated with drusen from a single AMD donor revealed that IgG isotype 2a predominated.

Data described herein indicate that dendritic cells belonging to the DC1 family, and associated immune- and inflammatory-mediated processes are intimately associated with the process of drusen development. Such drusen-associated dendritic cells have been further characterized and a number of additional markers have been determined. Drusen core-associated epitopes include CD1a, CD4, CD11a, CD11c, CD14, CD45, CD64, CD68, CD83, CD86, and HLA-DR, and perhaps CD18 and CD40. Dendritic cells at both early and late stages of maturation are associated with drusen. Analyses of 179 drusen labeled with HLA-DR, CD1a, and CD86 revealed that cores are associated with approximately 40% of drusen. Similarly, confocal laser scanning of HLA-DR immunoreactivity of 104 drusen from aldehyde-fixed eyes revealed 44% of drusen with cores. Taken together, these data indicate that cores are a frequent occurrence in drusen, and are not a rare or isolated observation. In addition, the numbers of drusen-associated dendritic cells are significantly higher at earlier ages (40-50 years of age) when drusen precursors are just beginning to develop.

Complement Process: The full spectrum of complement proteins and inhibitors, as well as activated complement complexes, are present at the RPE-choroid interface based on immunohistochemical and biochemical analyses. Experiments have been conducted to determine whether inappropriate complement activation occurs within the sub-RPE space in individuals with AMD and whether such an aberrant process might have injurious effects on the RPE and/or choroidal cells and promote neovascularization. These experiments were directed toward assessing various parameters of this pathway. To date, numerous complement pathway proteins has been identified to be associated with drusen, Bruch's membrane, the basal surface of the RPE, and/or the sub-RPE space. These include CD4, CD11c, CD14, CD21, CD35, CD40, CD55, CD59, CD64, CD83, CD86, C1q, C1q inhibitor, C2, C3, C3a, C3d, C5a, factor H, factor I, factor B, SP40,40, and mannose binding protein.

In one study, the distribution of the C5b-9 terminal complement complex was examined in 30 human donor eyes ranging in age between less than 1 year and 94 years of age, with and without AMD. A strong correlation between intensity and distribution of C5b-9 associated with the choriocapillaris and a diagnosis of AMD was observed, indicating that the choriocapillaris of AMD patients may be under more rigorous attack than that of individuals without AMD. Gene expression data obtained using RT-PCR suggest a role for locally produced complement components in the activation of complement in the region of Bruch's membrane. Specifically, C3, C5, APP, clusterin, and Factor H are synthesized by the RPE (see also Mullins et al., FASEB J. 2000). C9 and MASP-1 are synthesized by adjacent choroidal and/or retinal cells and could contribute to complement activation in Bruch's membrane. Gene expression array data confirm that a majority of molecules involved in complement activation and inhibition are expressed locally by RPE and choroid cells.

Other studies have been conducted to reveal the mechanism(s) of complement activation in individuals with AMD. C1q—an indicator of the classical pathway—is not a major drusen constituent, although immunoglobulin has been identified within drusen using both biochemical and immunohistochemical approaches. Nearly all of the above major activators of the alternative pathway are present in the drusen and/or the RPE. Factor H, C3b and C3d are present in all drusen phenotypes, as is mannose binding protein, indicating that activation of the complement cascade at the level of the RPE-choroid interface may occur via the alternative and/or lectin pathways.

Moreover, biochemical studies have been conducted in order to determine whether C5b-9 complexes are inserted into plasma membranes of RPE and/or choroidal cells. ELISA and ELISA capture assays demonstrate that the C5b-9 MAC complexes are present, and predominant, in the plasma membranes isolated from the RPE and choroid samples from older donors with and without AMD. The highest levels of C5b-9 have been detected in cells from a donor with neovascular AMD.

### Example 17

### In Vitro Analyses of Choroidal Monocvtes/Dendritic Cells

Choroidal Cultures: A battery of choroidal cultures were generated as models for evaluating the biology and pathophysiology of resident choroid dendritic cells. Cells are detected that express HLA-DR and that develop elongated, highly HLA-DR-immunoreactive processes resembling the processes that penetrate Bruch's membrane in situ. These processes terminate in rounded bulbs that further resemble “drusen cores”.

Differentiation In Vitro: Cells in choroidal cultures that treated with Granulocyte-Monocyte Colony Stimulating Factor (GM-CSF) and interleukin-4 (IL-4) for 5-7 days were stimulated to express the mature dendritic cell antigen CD86, whereas few CD86 positive cells were detected in untreated cultures. These cells appeared either rounded, with extensive filopodia in a radial orientation, or as flattened cells with numerous veils and, often, with a single, elongated process.

Migration In Vitro: Additionally data indicate that choroidal antigen presenting cells may send processes through pores in the direction of a higher concentration of LPS. This observation is highly relevant to the generation of a culture model of choroidal DC pathophysiology in Bruch's membrane. Cells treated with 1 μg/mL on the opposite side of a porous membrane appeared to send HLA-DR positive processes into the 0.4 μm pores in a Falcon insert. Further studies can verify this behavior and to characterize the factors that may induce DC process growth and its effects on the underlying RPE in vivo.

### Example 18

### Distribution of Choroidal Leukocytes

Various studies were performed to examine distribution of choroidal leukocytes. The following summarized the results.

CD1a Labeling: Labeling of the dendritic cell marker CD1a was strongly and consistently associated with drusen cores (and pre-drusen bodies) and also with rare choroidal cells possessing a dendritic morphology, as well as in drusen cores. Only very rarely was the cell body of the drusen core-associated cell labeled with CD1a antibodies.

CD3 Labeling: Labeling of numerous choroidal cells, distributed from the CC to outer choroid, was detected with antibodies directed against the lymphocyte marker CD3. Weak labeling of cores was detected with one clone, but not with another clone.

CD4 Labeling: Labeling of numerous choroidal cells, distributed from CC to outer choroid, was detected with anti-CD4 antibodies. In addition, drusen cores were rarely labeled, even when they colocalized with CDIa labeling in adjacent sections.

CD8 Labeling: Choroidal cells that are reactive with CD8 antibodies were observed commonly. These cells were spherical or flattened. The flattened cells lined up along Bruch's membrane!! Drusen cores were not bound by anti-CD8 antibody, whereas cores were detected with CD1a in adjacent sections.

CD11a Labeling: Labeling of large numbers of choroidal leukocytes was observed with a monoclonal antibody directed against CD11a. In addition, strong labeling of drusen cores was observed. These cores were frequently contiguous with labeled cell bodies in the choroid.

CD11c Labeling: In contrast to CD11a, very few positive cells were observed with CD11c antibodies. Drusen cores were strongly reactive, however. This labeling was not inhibited by preincubation of sections with an excess of human immunoglobulin. In addition, diffuse regions of the choroid exhibited positive labeling.

CD14 Labeling: Labeling of choroidal and scleral cells was common observed with two antibodies directed against the monocyte marker CD14. Drusen cores were also labeled, although their associated choroidal cell bodies were not reactive.

CD15 Labeling: The granulocyte marker CD15 was not detected in drusen cores, although vascular leukocytes exhibited immunoreactivity.

CD45 Labeling: Labeling of choroidal cells with two monoclonal antibodies directed against the leukocyte common marker CD45 was similar to the pattern observed with CD11a antibodies. Large numbers of choroidal leukocytes were labeled, as were drusen cores.

CD64 Labeling: The Fc receptor CD64 exhibited a similar pattern of immunoreactivity as CD14, with choroidal cells and some scleral cells being positive, in addition to drusen cores. The connecting isthmus between the core and the choroidal cell has not been visualized with this antibody.

CD68 Labeling: Antibodies directed against the monocyte marker CD68 labeled drusen cores and their associated choroidal cell bodies. They also labeled choroidal cells that are distributed between the CC and the outer choroid.

CD83 Labeling: CD83 antibodies labeled drusen cores and, less intensely, drusen core-associated choroidal cell bodies. In addition, widespread labeling of cells with CD83 was observed in the retina, choroid, and sclera using a monoclonal antibody directed against the dendritic cell marker CD83. Muller cells in the retina, as well as large numbers of choroidal cells and drusen cores were positively labeled with this antibody. Radial labeling of choroidal arteries was also observed with this antibody, most likely corresponding to smooth muscle cells. Following preincubation with an excess of human immunoglobulin, labeling was restricted to drusen cores, including tiny, “predrusen” lesions.

CD86 Labeling: Antibodies directed against CD86, a marker of dendritic cells, memory cells, and some T cell clones, was expressed within drusen cores. In addition, choroidal and scleral cells, from the choriocapillaris to the outer sclera, expressed this protein.

HLA-DR Labeling: Immunoreactivity of drusen with two monoclonal antibodies directed against the MHC class I antigens HLA-DR (clone TAL. 1B5) and HLA-DR/DP/DQ (clone CR3/43) revealed a number of distinct but overlapping patterns of labeling. Large numbers of choroidal cells possessing an extended morphology were observed, as well as cells in two layers of the inner and outer sclera, and weak labeling of choroidal endothelial cells. Four patterns of drusen labeling were detected: (1) unlabeled drusen (approximately 12%, primarily drusen of a highly round, “cuticular” morphology); (2) labeling of drusen cores, with or without the associated choroidal cell body; (3) labeling of drusen cores with less intense, more diffuse labeling surrounding the core; and (4) labeling of entire drusen.

### Example 19

### Mouse Model of Drusen Biogenesis, Immune-Mediated Processes, and/or AMD

The transfer of human cells into mice with SCID background has been proven to be a useful model for immunologic studies, but has not been employed previously as a method for studying drusen biogenesis and its associated immune-mediated processes.

Mice homozygous for the SCID (severe combined immunodeficiency) mutation lack functional T- and B-cells and macrophages, and hence fail to generate either humoral or cell-mediated immunity. The absence of T and B-cells which normally mediate xenograft rejection enables SCID mice to support variable levels of growth of human lymphohematopoetic cells (LHPC). The Emv30^{null}NOD-scid mouse strain (The Jackson Laboratory, Bar Harbor, Main) has been demonstrated to be an improved host for adaptive transfer of autoimmune diabetes and growth of human LHPC. This strain has the advantage of a higher level of human LHPC growth than the C.B-17-scid strain in which the scid mutation originated. When used as a host in passive transfer experiments and for the repopulation with human cells various SCID mice have provided great insight to the contribution of T-cells and/or autoantibodies in various autoimmune diseases.

Human PBLs or enriched population of monocyte/dendritic cells will be obtained by leukapheresis or by gradient density centrifugation (followed by GM-CSF and/or IL-4 incubation for the purpose of enriching the dendritic cell population) from the peripheral blood of AMD patients and from healthy volunteers. In some experiments, DCs will be pre-incubated with either RPE cells (with or without AMD) or isolated fractions of Bruch's membrane. For each experiment, aliquots 2×107 PBLs from a single human donor will be injected into aged matched female mice, ages 6-8 weeks. Injections will be performed either interperitoneally (i.p.) or interocularly. In experiments with serum antibody transfer, serum Ig will be purified using protein A or G columns and Ig in concentration of 1 mg/ml in PBS will be injected i.p. (200mg/kg/mouse).

Four weeks after the injection, splenic leukocytes from all human PBL recipients will be phenotyped by FACS analysis with commercially available monoclonal antibodies that will identify the total numbers of human LHPC (CD45+), as well as the proportion of these cells comprised by macrophages (CD 14+), B-cells (CD19+), T-cells (CD3+), and dendritic cells (CD83+, CD86+, CD11a+). The relative proportions of the total T-cell population comprising the CD4+and CD8+subsets will be also assessed.

The effects of PBL transfer to injected mice will be evaluated by a number of functional, histologic, biochemical, and immunocytochemical assays. The lymphocyte proliferation assay (LPA) will be used to evaluate T-cell immunoreactivity against RPE/drusen proteins. ELISA using goat anti-human Ig (A, G and M) will determine levels, specificity, titer and isotype of human antibodies in mice sera. Mouse eyes will be examined for the presence of histopathological changes and set of anti-human antibodies (directed against MHC class-II antigens and various, cell-specific CD antigens) will be employed for immunocytochemical localization of human immunocompetent cells and Ig in mouse eye tissue.

1. A method for diagnosing or identifying a predisposition to the development of a drusen associated ocular disorder in a subject, comprising detecting at least one drusen associated marker (DRAM).

2. The method of claim 1, wherein said drusen associated ocular disorder is selected from the group consisting of macular degeneration, North Carolina macular dystrophy, Sorsby's fundus dystrophy, Stargardt's disease, pattern dystrophy, Best disease, dominant drusen, and radial drusen.

3. The method of claim 2, wherein the drusen associated ocular disorder is age-related macular degeneration.

4. The method of claim 1, further comprising examining of the subject with an ophthalmologic procedure.

5. The method of claim 1, wherein the drusen associated marker is a phenotypic marker.

6. The method of claim 5, wherein the marker is selected from the group consisting of RPE dysfunction and/or death, immune mediated events, dendritic cell activation, dendritic cell migration and differentiation, extrusion of the dendritic cell process into the sub RPE space, and the presence of geographic atrophy or disciform scars.

7. The method of claim 1, wherein the drusen associated marker is a genotypic marker.

8. The method of claim 1, wherein the drusen associated marker is selected from the group consisting of:
(a) a marker involved in immune mediated events associated with drusen formation;
(b) a marker involved in RPE dysfunction and/or death;
(c) a marker expressed by choroidal and RPE cells;
(d) a molecule associated with drusen;
(e) a marker of drusen-associated dendritic cells;
(f) a dendritic cell-associated accessory molecule that participate in T cell recognition;
(g) a marker associated with dendritic cell expression;
(h) a marker associated with dendritic cell proliferation; and
(i) a marker associated with dendritic cell differentiation.

9. The method of claim 1, wherein the drusen associated marker is indicative of an immune mediated process at the RPE-Bruch's membrane-choroid interface.

10. The method of claim 9, wherein the drusen associated marker is an autoantibody.

11. The method of claim 10, wherein the autoantibody is an autoantibody directed against drusen, an autoantibody directed against RPE, an autoantibody directed against a B cell, an autoantibody directed against a T cell, an autoantibody directed against a macrophage, an autoantibody directed against a dendritic cell, an autoantibody directed against a systemic antigen, or an autoantibody directed against a neoantigen.

12. A method for treating or preventing the development of a drusen associated ocular disorder in a subject, comprising providing to the subject an effective amount of an agent which inhibits immune cell migration, proliferation, or differentiation.

13. The method of claim 12, wherein said immune cell is a dendritic cell or a dendritic cell precursor.

14. The method of claim 13, wherein said agent inhibits dendritic cell migration and extension of dendritic cell process through Bruch's membrane and/or into the sub-retinal pigment epithelial.

15. The method of claim 12, wherein said agent inhibits initiation or maintenance of a cellular or humoral immune response.

16. The method of claim 12, wherein said agent disrupts antigen presentation and dendritic cell-T cell interaction.

17. The method of claim 12, wherein said immune cell is a T cell or a B cell.

18. The method of claim 12, wherein said agent inhibits migration of the immune cell and is an agonist of a cytokine selected from the group consisting of GMCSF, TNIFα, and IL-1.

19. The method of claim 12, wherein said agent inhibits proliferation of the immune cell and is selected from the group consisting of antagonists of GMCSF, IL-4, IL-3, SCF, FLT-3, and TNFα.

20. The method of claim 12, wherein said agent inhibits differentiation of the immune cell and is selected from the group consisting of IL-10, M-CSF, IL-6, and IL-4.

21-24. (canceled)
